(19) United States
(12) Patent Application Publication
(10) Pub. No.: US 2022/0249073 A1
(43) Pub. Date: Aug. 11, 2022

(54) INTEGRATED SAMPLE COLLECTION AND TEST DEVICE

(71) Applicant: Orthogonal Diagnostics, Inc., San Diego, CA (US)

(72) Inventors: Andrew Freestone CHARLTON, San Diego, CA (US); Jonathan Edward AVILA, San Jose, CA (US); Robert Elroy KLEPPER, San Diego, CA (US)

(21) Appl. No.: 17/576,346

(22) Filed: Jan. 14, 2022

Related U.S. Application Data
(60) Provisional application No. 63/146,803, filed on Feb. 8, 2021.

(51) Int. Cl. A61B 10/00; G01N 1/28; G01N 33/52

(52) U.S. Cl. CPC A61B 10/0096; A61B 10/0045; G01N 2001/2826; G01N 33/523; A61B 2010/009; G01N 1/2813

(57) ABSTRACT

The technology in part relates to diagnostic test devices and in part relates to test devices that include a lateral flow test strip. The technology in part relates to a test device system that incorporates sample collection, delivery, measuring, processing, incubation, flow regulation and analyzing a sample of interest in a single assembly.

### RELATED PATENT APPLICATION

This patent application claims the benefit of U.S. provisional patent application No. 63/146,803 filed on Feb. 8, 2021, entitled INTEGRATED SAMPLE COLLECTION/TEST DEVICE INCORPORATING SAMPLE COLLECTING, DELIVERING, MEASURING, PROCESSING, INCUBATING AND ANALYZING IN A SINGLE DEVICE, naming Andrew Charlton et al. as inventors, and designated by attorney docket no. ODI-1001-PV. The entire content of the foregoing patent application is incorporated herein by reference for all purposes, including all text, tables and drawings.

### FIELD

The technology in part relates to diagnostic test devices and in part relates to test devices that include a lateral flow test strip. The technology in part relates to a test device system that incorporates sample collection, delivering, measuring, processing, incubation, flow regulation and analyzing a sample of interest in a single assembly.

### BACKGROUND

Analytical testing of a sample involves multiple steps including collecting, delivering, measuring, processing, incubating and analyzing a sample of interest. Sample collection and processing usually require special and expensive equipment and trained personnel to execute the processes usually only available in laboratories. After a sample is collected, it often is shipped to a lab, processed prior to testing and analyzed. A laboratorian will take a specific volume from the sample, mix it with reagents, incubate for a predetermined amount of time, and then deliver the sample to a test device, analyzer or reaction matrix where the sample is analyzed and the results determined, either visually or by an instrument. Depending on the target molecules to be analyzed, the sample may need to be split and processed on multiple testing platforms.

### SUMMARY

Provided in certain aspects are integrated devices that incorporate collection, processing, metering, incubation, flow restriction and analysis of a sample. In certain aspects, provided is a device that includes a sample collection device component and a test device component for processing the sample. Contained in a device provided herein are components for filtration, reagent/chemistry delivery, incubation, flow restriction and sample analysis. A device provided herein generally is self-contained, and provides integrated sample collection, sample metering, multiple sample processing steps, flow restriction and sample testing. A device provided herein is capable of assessing various sample types for the presence or absence and/or amount of one or more targeted markers and/or analytes. A device provided herein generally processes a sample as an integrated unit prior to analysis of the sample, and allows for interrogation of results visually or via a reader-based system.

Provided in certain aspects is a sample collection device that includes: a handle, a stem in connection with the handle, and an absorbent sample pad in connection with the stem, where the sample pad includes a proximal surface, a distal surface and an exterior side surface between the proximal surface and the distal surface; and at least a portion of the sample pad extends from the stem.

Provided also in certain aspects is a test device that includes a hollow test device housing that includes a proximal terminus, a distal terminus, a sidewall between the proximal terminus and distal terminus, an interior, and a window disposed in the sidewall; a cartridge disposed within the test device housing, the cartridge that includes: a hollow cartridge housing comprising a proximal terminus distally disposed from, and adjacent to, the test device housing proximal terminus, a distal terminus, a sidewall disposed between the proximal terminus and the distal terminus, an interior, and a terminal wall disposed at the distal terminus, and a hollow terminal projection member extending from the terminal wall of the cartridge housing; the terminal projection member including a proximal terminus disposed on the terminal wall, a distal terminus opposite the terminal wall, a sidewall disposed between the proximal terminus and distal terminus, and an interior; a porous transfer pad disposed within the terminal projection member of the cartridge; and a lateral flow strip disposed in the test device housing adjacent to the transfer pad and distally disposed from the cartridge; the test strip including a proximal surface, a distal surface, and an anterior surface and an opposite posterior surface each disposed between the proximal surface and the distal surface; where the window in the test device housing opposes a portion of the strip anterior side.

Also provided in certain aspects is a composition or kit that includes a sample collection device described herein and a test device described herein, where the sample collection device is separated from the test device. Provided also in certain aspects is an assembly that includes a sample collection device described herein and a test device described herein, where the sample collection device is joined to the test device. Also provided in certain aspects is a method for preparing an assembly that includes joining a sample collection device described herein to a test device described herein. Provided also herein is a method for determining presence or absence and/or amount of an analyte in a sample, which includes detecting a detectable signal from the lateral flow strip through the window of a test device described herein in an assembly with a sample collection device described herein; and determining the presence or absence and/or amount of the analyte in the sample from the detectable signal.

Provided also herein in certain aspects is a method for manufacturing a sample collection device that includes injecting a liquid polymer into a mold; hardening the polymer in the mold, where the sample collection device or portion thereof is formed in the mold; and separating the sample collection device or portion thereof from the mold. Also provided herein in certain aspects is a method for manufacturing a sample collection device that includes providing a portion of the sample collection device including the handle and stem; and providing the sample pad and joining the sample pad to the stem. Provided also herein in certain aspects is a method for manufacturing a test device that includes injecting a liquid polymer into a mold; hardening the polymer in the mold, where the test device anterior member, posterior member or cartridge is formed in the mold; separating the test device anterior member, posterior member or cartridge from the mold. Also provided herein in certain aspects is a method for manufacturing a test device that includes: providing a test device housing posterior member and a test device housing anterior member; contacting the cartridge with the test device housing posterior member and/or the test device housing anterior member; and joining the test device posterior member and the test device anterior member, whereby the cartridge is contained within the test device housing interior. Provided in certain aspects is a mold for manufacturing a sample collection device or test device described herein.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 is a perspective view of a test device 100 and a separate sample collection device 200.

FIG. 2 is an enlarged perspective view of sample collection device 200.

FIG. 3A is a side view of sample collection device 200, FIG. 3B is an opposing side view thereof, FIG. 3C is a front view thereof, FIG. 3D is a rear view thereof, FIG. 3E is a top view thereof and FIG. 3F is a bottom view thereof. FIG. 3G is a section view of sample collection device 200 through cutting plane A-A shown in FIG. 3D.

FIG. 4A is side view of test device 100, FIG. 4B is an opposing side view thereof, FIG. 4C is a front view thereof, FIG. 4D is a rear view thereof, FIG. 4E is a top view thereof, and FIG. 4F is a bottom view thereof.

FIG. 5 is a perspective and exploded view of test device 100.

FIG. 6 is an enlarged perspective view of the test device housing anterior member 103.

FIG. 7 is an enlarged perspective view of the test device housing posterior member 102.

FIG. 8 is a perspective and exploded view of cartridge 170 and certain pad components.

FIG. 9 is an enlarged perspective view of cartridge 170 without pad components.

Each of FIG. 10 and

FIG. 11 is a section view of test device 100 through cutting plane B-B shown in FIG. 4D.

FIG. 12A is an enlarged view of the region the region of test device 100 delineated by the broken line circle E depicted in FIG. 10. FIG. 12B is an enlarged view of the region of test device 100 delineated by the broken-line circle D shown in FIG. 10.

FIG. 13 is a perspective view of assembly 300 containing test device 100 and sample collection device 200.

FIG. 14A is a side view of test assembly 300, FIG. 14B is an opposing side view thereof, FIG. 14C is a front view thereof, FIG. 14D is a rear view thereof, FIG. 14E is a top view thereof, and FIG. 14F is a bottom view thereof.

FIG. 15 is a section view of assembly 300 through cutting plane C-C shown in FIG. 14D.

FIG. 16A is a side view of sample collection device 200 when not joined with test device 100 and FIG. 16B is a side view of sample collection device 200 when joined with test device 100, where test device 100 is shown in broken-line form.

FIG. 17 is a perspective view of assembly 600 that includes test device 400 and sample collection device 500.

FIG. 18 is a side view of test device 400 not joined to sample collection device 500.

FIG. 19 is an enlarged perspective view of sample collection device 500 and

FIG. 20 is an enlarged perspective view of cartridge 470 disposed in the test device 400.

FIG. 21A to FIG. 21E illustrate assembly 900 that includes test device 700 and sample collection device 800. FIG. 21A is a top view of assembly 900, FIG. 21B is a side view thereof, FIG. 21C is a bottom view thereof, FIG. 21D is a front view thereof and FIG. 21E is a rear view thereof.

FIG. 22 is an exploded view of assembly.

FIG. 23 is a perspective view of assembly 1200 that includes test device 1000 and sample collection device 1100.

FIG. 24A is a top view of assembly 1200, FIG. 24B is a side view thereof, FIG. 24C is a bottom view thereof, FIG. 24D is a front view thereof and FIG. 24E is a rear view thereof.

FIG. 25A is an enlarged section view of assembly 1200 through cutting plane D-D shown in FIG. 24E, for a region delineated by the broken-line circle F shown in FIG. 24B, where button 1029 is not depressed. FIG. 25B is a view similar that shown in FIG. 25A where button 1029 is depressed.

Provided in the following table is a listing of elements identified in the drawings.

| Callout | Element |
|---|---|
| 100 | test device |
| 101 | test device housing |
| 102 | test device housing posterior member (also referred |
| | to as test device housing (bottom)) |
| 103 | test device housing anterior member (also referred |
| | to as test device housing (top)) |
| 104 | junction between elements 102 and 103 |
| 105, 105a, | test device housing proximal terminus |
| 105b | |
| 106, 106a, | test device housing distal terminus |
| 106b | |
| 107, 107a, | base projection |
| 107b | |
| 108a, 108b, | test device housing sidewall exterior surface |
| 108c, 108d | |
| 109a, 109b, | test device housing sidewall exterior surface |
| 109c | |
| 110 | test device housing window (also referred to as |
| | results viewing window) |
| 112, 112a, | recess in test device housing sidewall around window |
| 112b | |
| 113 | test device housing anterior member recessed protrusion |
| 113a | test device housing anterior member recessed protrusion |
| | edge |
| 113b | test device housing anterior member surface adjacent to |
| | recessed protrusion |
| 114a, 114b, | connector |
| 114c | |
| 115 | connector counterpart |
| 116 | posterior member edge |
| 117 | recessed edge |
| 118 | sidewall between elements 116 and 117 |
| 119a, 119b | connector post |
| 120 | test device housing proximal opening (also referred to |
| | as sample collector port) |
| 121a, 121b, | test device housing anterior member sidewall interior |
| 121c | surface |
| 122 | test device housing annular recessed surface |
| 123 | test device housing annular protrusion |
| 125 | test device housing proximal opening rim |
| 126 | test device housing second extension wall |
| 126′ | second extension wall posterior edge |
| 126″ | second extension wall side edge |
| 127 | extension bar |
| 127′ | extension bar posterior edge |
| 127″ | extension bar distal side |
| 127′″ | extension bar side edge |
| 128 | interior window projection |
| 130 | sample expression zone (containing a compression zone) |
| 131a, 131b | test device housing posterior member sidewall interior |
| | surface |
| 132 | test device housing annular recessed surface |
| 133 | test device housing annular protrusion |
| 134 | test device housing lumen |
| 135 | test device housing proximal opening rim |
| 145 | first cartridge housing pad (also referred to as |
| | filtration/chemistry pad) |
| 145a | first cartridge housing pad proximal surface |
| 145b | first cartridge housing pad distal surface |
| 145c | first cartridge housing pad side surface |
| 146 | optional second cartridge housing pad (also referred to |
| | as conjugate/chemistry pad) |
| 146a | second cartridge housing pad proximal surface |
| 146b | second cartridge housing pad distal surface |
| 146c | second cartridge housing pad side surface |
| 150 | sample transfer zone |
| 151 | test device housing sidewall interior surface |
| 152 | third extension wall |
| 152′ | third extension wall proximal edge |
| 152″ | third extension wall anterior edge |
| 153 | fourth extension wall |
| 154 | first extension wall |
| 154′ | first extension wall anterior edge |
| 154″ | first extension wall side |
| 165 | transfer pad (also referred to as transport pad) |
| 166 | transfer pad proximal terminus (proximal surface) |
| 167 | transfer pad distal terminus (distal surface) |
| 168a | transfer pad anterior surface |
| 168b | transfer pad posterior surface |
| 169 | transfer pad side surface |
| 170 | test device cartridge housing (also referred to as a |
| | sample process cartridge housing) |
| 171 | cartridge housing proximal terminus |
| 171a | cartridge housing proximal edge |
| 172 | cartridge housing distal terminus |
| 173 | cartridge housing sidewall |
| 173a | cartridge housing sidewall exterior surface |
| 173b | cartridge housing sidewall interior surface |
| 173c | cartridge housing sidewall transition |
| 173d, 173e | cartridge housing lumen |
| 174 | cartridge terminal projection |
| 175 | cartridge housing terminal wall (also referred to as |
| | a restriction structure) |
| 175a | cartridge housing terminal wall exterior surface |
| 175b | cartridge housing terminal wall interior surface |
| 176a | cartridge terminal projection proximal terminus |
| 176b | cartridge terminal projection distal terminus |
| 176c | cartridge terminal projection posterior side member |
| | exterior surface |
| 176d | cartridge terminal projection anterior side member |
| | exterior surface |
| 176e | cartridge terminal projection side member exterior |
| | surface |
| 177a, 177b | cartridge housing terminal wall bore (also referred |
| | to as a sample transfer port) |
| 178a | cartridge housing proximal recessed surface |
| 178b | cartridge housing proximal terminus rim surface |
| 178c | annular groove between recessed surface 122, 132 and |
| | recessed surface 178a with rim surface 178b floor |
| 179 | cartridge terminal projection interior |
| 180 | sample readout zone |
| 181a, 181b | containment post |
| 182a, 182b, | support rib |
| 182c, 182d | |
| 182a′, 182b′, | support rib edge |
| 182c′, 182d′ | |
| 183a, 183b, | connector member |
| 183c, 183d | |
| 184a, 184b, | connector member receptacle |
| 184c, 184d | |
| 185 | test strip (also referred to as a lateral flow test strip) |
| 185a | test strip proximal terminus |
| 185b | test strip distal terminus |
| 185c | test strip anterior surface |
| 185d | test strip posterior surface |
| 186, 186a | test device housing sidewall interior surface |
| 187 | test device housing lumen |
| 188 | distally-disposed test strip absorbent pad |
| 194 | transverse axis |
| 195 | cylindrical region of test device housing |
| 196 | lateral arcoid cylindrical segment region of test device |
| | housing |
| 197 | lateral planar cylindrical segment region of test device |
| | housing |
| 198 | lateral axis in in proximal to distal direction |
| 199 | longitudinal axis in posterior to anterior direction |
| 200 | sample collection device (also referred to as sample |
| | collector) |
| 201 | sample collection device handle |
| 202 | sample collection device handle proximal terminus |
| 203 | sidewall between elements 202 and 204 (exterior surface) |
| 204 | sample collection device handle distal terminus |
| 205 | sample collection device handle sidewall (interior |
| | surface) |
| 206 | sample collection device handle interior step |
| 210 | sample collection device stem |
| 212 | stem proximal terminus |
| 213 | stem distal terminus |
| 214 | stem sidewall (exterior surface) |
| 215 | stem sealing member |
| 216 | stem sidewall (interior surface) |
| 217 | stem sealing member |
| 218 | stem annular protrusion (also referred to as click lock |
| | feature) |
| 220 | stem transverse interior wall |
| 221 | stem transverse interior wall distal surface |
| 222 | stem transverse interior wall proximal surface |
| 223 | stem lumen |
| 224 | stem sidewall (interior surface) |
| 230 | sample pad (also referred to as absorbent collection pad) |
| 232 | sample pad side surface |
| 234 | sample pad distal terminus (distal surface) |
| 235 | sample pad proximal terminus (proximal surface) |
| 290a, 290b | lateral length of sample pad |
| 300 | assembly of test device 100 and sample collection device |
| | 200 |
| 400 | test device having screw connector member |
| 470 | test device cartridge |
| 471 | cartridge housing proximal terminus |
| 472 | cartridge housing distal terminus |
| 473b | cartridge housing sidewall interior surface |
| 473d | cartridge housing lumen |
| 474 | cartridge terminal projection |
| 476b | cartridge terminal projection distal terminus |
| 479a | test device cartridge shank |
| 479b, 479c | shank thread |
| 479d | test device cartridge collar |
| 479e | collar exterior surface |
| 500 | sample collection device having screw connector member |
| | counterpart |
| 501 | sample collection device handle |
| 502 | sample collection device handle proximal terminus |
| 503 | sidewall between elements 502 and 504 (exterior surface) |
| 504 | sample collection device handle distal terminus |
| 507 | sample collection device handle interior surface |
| 508 | sample collection device handle void |
| 509 | sample collection device handle thread |
| 510 | sample collection device stem |
| 513 | stem distal terminus |
| 514 | stem sidewall (exterior surface) |
| 515 | stem sealing member |
| 517 | stem sealing member |
| 530 | sample pad (also referred to as absorbent collection pad) |
| 532 | sample pad side surface |
| 534 | sample pad distal terminus (distal surface) |
| 600 | assembly of test device 400 and sample collection device |
| | 500 |
| 700 | test device having tab separator |
| 702 | test device housing posterior member |
| 703 | test device housing anterior member |
| 707 | test device housing base extension |
| 710 | test device housing window |
| 770 | test device cartridge housing |
| 745 | first cartridge housing pad |
| 746 | second cartridge housing pad |
| 765 | transfer pad (also referred to as transport pad) |
| 768b | transfer pad posterior surface |
| 774 | cartridge terminal projection |
| 785 | test strip |
| 785c | test strip anterior surface |
| 790 | barrier (pull tab) |
| 791 | slot |
| 800 | sample collection device |
| 900 | assembly of test device 700 and sample collection device |
| | 800 |
| 1000 | test device having depressible button |
| 1002 | test device housing posterior member |
| 1003 | test device housing anterior member |
| 1007 | test device housing base extension |
| 1010 | test device housing window |
| 1029 | test device housing button |
| 1029a, 1029b, | test device housing button perimeter slit |
| 1029c | |
| 1029d | test device housing button posterior terminus |
| 1029e | test device housing button catch |
| 1029f | test device housing button catch counterpart (disposed |
| | in/on extension bar) |
| 1029g | test device housing extension bar |
| 1045 | first cartridge housing pad |
| 1046 | second cartridge housing pad |
| 1065 | transfer pad (also referred to as transport pad) |
| 1068a | transfer pad anterior surface |
| 1068b | transfer pad posterior surface |
| 1070 | test device cartridge housing |
| 1074 | cartridge terminal projection |
| 1085 | test strip |
| 1085c | test strip anterior surface |
| 1100 | sample collection device |
| 1200 | assembly including test device 1000 and sample |
| | collection device 1100 |

### DETAILED DESCRIPTION

Collected samples may require preconditioning, measuring a fixed volume of the sample and incubating the sample prior to testing the sample on a lateral flow device. A device described herein provides the capability to collect a desired sample, filter the sample if required, precondition the sample, extract a measured amount of the sample, mix it with chemical reagents, incubate the mixture for a period of time to ensure proper test reaction or coupling and deliver the sample mixture to a test element where the analysis for the presence or absence and/or amount of one or more analytes (i.e., one or more target molecule) is determined. The completed test element then can be interrogated visually or using a reader system within a matter of minutes. A test element typically is a lateral flow test strip. These steps are carried out within a test device, using the sample collection device to not only collect the sample, but to apply the force to carry out the manipulations. A device described herein reduces the complexity of the processes so that an individual with minimal training can execute the required steps from sample collection to final result.

### Sample Collection Device Component

Provided in certain implementations is a sample collection device component that includes: a handle, a stem in connection with the handle, and an absorbent sample pad in connection with the stem, where: the sample pad includes a proximal surface, a distal surface and an exterior side surface between the proximal surface and the distal surface; and at least a portion of the sample pad extends from the stem distal terminus. A sample pad can be joined to the stem in any suitable manner, including without limitation by an interference fit or a weld (e.g., glue or sonic weld). In certain instances, a stem includes an interior and a portion of a sample pad is disposed within the stem interior. In certain instances, a sample pad is not inserted into a stem and the proximal terminus of the pad is fused to the stem.

A handle and stem of a sample collection device can be composed of any suitable material, such as a polymer or polymer blend, for example. In certain instances, a handle and a stem of a sample collection device independently include polypropylene, polyethylene, acrylonitrile butadiene styrene, or a combination of two or more of the foregoing. A handle member can be hollow or not hollow, can be tubular and can be a frustum or a cylinder. A stem member independently can be hollow or not hollow, can be tubular, and can be a frustum or a cylinder.

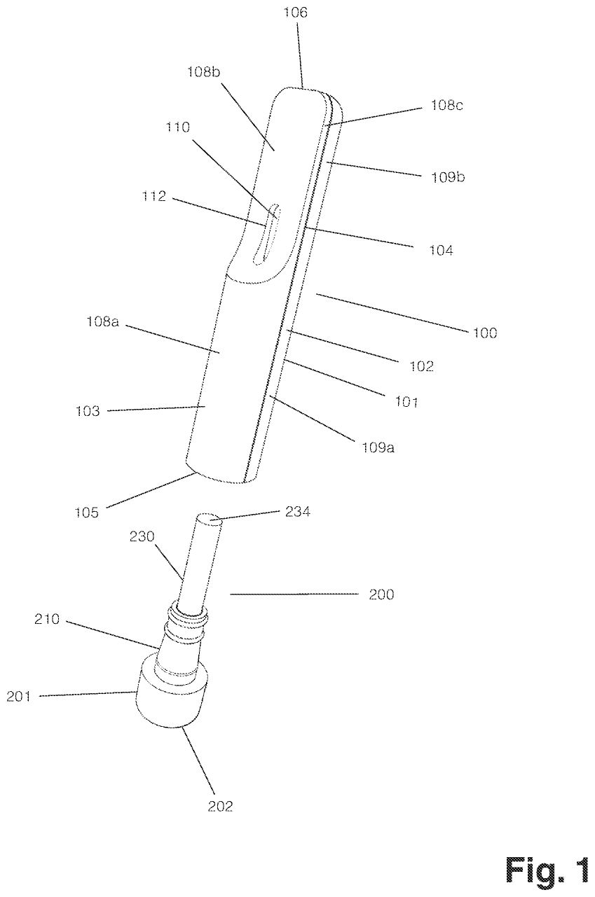

A sample collection device can include any suitable sample pad for absorption of sample fluid and delivery of the fluid into a test device. A sample pad of a sample collection device often is compressible. The lateral length of a sample pad is parallel to lateral axis 198 shown in FIG. 10, where the sample collection device is in the same orientation shown in FIG. 1 with respect to the test device. The lateral length of a sample pad sometimes is the major dimension of the sample pad, in which instance the lateral length is greater than the width of the proximal surface and distal surface of the sample pad. The width of the proximal surface and the distal surface of the sample pad is a diameter for instances in which the sample pad is cylindrical. The lateral length of a sample pad sometimes is at least 1.5-, 2-, 2.5-, 3-, 3.5-, 4-, 4.5-, 5-, 5.5-, 6-, 6.5-, 7-, 8-, 9- or 10-times greater than the width of the proximal surface and/or the distal surface of the sample pad. A sample pad can be laterally compressible (i.e., the lateral length of a sample pad is compressible and can be shortened), as described herein with respect to assemblies of a sample collection device component and a joined test device component.

In certain implementations, a sample pad has a retention volume (e.g., absorbs a maximum fluid volume) of about 0.1 milliliters to about 10 milliliters, or about 0.25 milliliters to about 2.5 milliliters, or about 1 milliliter to about 2 milliliters; or about 1.4 milliliters to about 1.7 milliliters, or about 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 28.8, 2.9, 3, 4, 5, 6, 7, 8, 9 or 10 milliliters. In certain implementations, a sample pad has a density of about 0.01 grams/cubic centimeter (g/cc) to about 1 g/cc, or about 0.05 g/cc to about 0.8 g/cc, or about 0.05 g/cc to about 0.6 g/cc, or about 0.3 g/cc to about 0.4 g/cc, or about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9 or 1 g/cc. A sample pad can include (i) polyethylene, or (ii) polypropylene, or (iii) polyethylene and polypropylene, in certain instances. A sample pad sometimes has a length of about 15 millimeters (mm) to about 55 mm (e.g., about 25 mm to about 45 mm; about 30 mm to about 40 mm; or about 35 mm) and an outer diameter of about 1 mm to about 15 mm (e.g., about 6 mm to about 12 mm; about 8 mm). A sample pad sometimes has an average (e.g., mean, median) pore size (e.g., diameter) of about 5 micrometers to about 60 micrometers, or about 10 micrometers to about 50 micrometers, or about 20 micrometers to about 40 micrometers, or about 30 micrometers to about 35 micrometers (e.g., about 5, 10, 15, 20, 25, 30, 32, 33, 35, 40, 45, 50, 55 or 60 micrometers), sometimes with a minimum pore size (e.g., pore diameter) of about 0.1 micrometers to about 10 micrometers, or about 0.5 micrometers to about 5 micrometers, or about 0.8 micrometers to about 2 micrometers (e.g., 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.5, 4.0, 4.5, 5.0, 5.5, 6.0, 6.5, 7.0, 8.0, 9.0 or 10 micrometers), and/or sometimes with a maximum pore size (e.g., pore diameter) of about 10 micrometers to about 300 micrometers, or about 50 micrometers to about 200 micrometers, or about 80 micrometers to about 110 micrometers (e.g., about 10, 20, 30, 40, 50, 60, 70, 80, 90, 95, 100, 120, 140, 160, 180, 200 or 300 micrometers). A sample pad of a sample collection device, and optionally pad(s) of a test device, sometimes each is unitary and does not include a bore. A sample pad sometimes is a single pad, and sometimes does not include multiple pads. In certain implementations, a sample pad is an assembly of multiple pads.

A sample pad of a sample collection device sometimes includes one or more reagents. In certain instances, the one or more reagents are chosen from a buffering agent, detergent, protein, enzyme, antibody or antigen-binding fragment thereof, antigen, binding pair member, bulking agent, water-absorbing agent, saccharide, polysaccharide, nucleic acid amplification reagent, nucleic acid and aptamer. The one or more reagents sometimes are distributed on and/or in all or a portion of a sample pad.

In certain implementations, a property of the sample pad changes upon contact with a fluid (e.g., a sample fluid), which change indicates to an operator that a sufficient amount of sample fluid has been absorbed by the sample pad. Such a changeable property sometimes is referred to as a sufficiency indicator. A sample pad can contain a colormetric agent that shifts color upon contact with fluid in certain instances. A colormetric agent can be disposed at a zone of the sample pad, which sometimes is referred to herein as an observation zone. A zone can be a transverse band circumferentially disposed in or on the sample pad in certain instances. A zone sometimes is disposed at the distal terminus or in a distal portion of a sample pad, and/or sometimes is disposed at the proximal terminus or proximal region of the sample pad. In certain implementations, a colormetric agent changes color upon contact with fluid in a sample pad. A colormetric agent can change from one color to higher intensity color or lower intensity color or no color, can change from one color to a different color, or can change from no detectable color to a detectable color, in certain instances. A sample collection device sample pad can include any suitable colormetric agent, and in certain implementations a colormetric agent is a dye (e.g., a triarylmethane dye (e.g., Brilliant blue FCF)) and/or a pH indicator. A colormetric agent sometimes is dehydrated on and/or in a sample pad and becomes hydrated upon sample pad uptake of a fluid. In certain implementations, a sample pad includes a structural material (e.g., a fiber) that changes color upon contact with a fluid (e.g., changes from colored to lower intensity color or colorless upon contact with a fluid). In certain implementations, a sample pad or portion thereof swells when contacted with a fluid.

In certain implementations, a handle and/or stem or portion thereof contains an observation zone. A handle and/or stem, or portion thereof, sometimes is transparent or translucent or open (i.e., cut-out section). A transparent or translucent or open section of the stem and/or handle can function as an observation zone or window that permits viewing of a colormetric agent or physical change by an operator. An operator can visually observe a colormetric change by eye in an observation zone, or observe a physical change visually or by touch in an observation zone, for example.

An observation zone can include one or more user-viewable indicator marks that can facilitate determination by an operator that a sufficient amount of sample fluid has been absorbed by the sample pad (e.g., a physical change or colormetric change at a particular indicator mark can provide an indication to an operator that a sufficient amount of sample fluid has been absorbed by the sample pad). In certain implementations, an observation zone is located in a stem member (e.g., described herein).

In certain instances, a dehydrated colormetric agent in a distal region of a sample pad hydrates, e.g., behind the stem wall, upon sample pad absorption of a fluid, and flows to an observation zone. In certain instances a hydrated colormetric agent can be visualized as a colored smear in an observation zone.

In certain instances, a portion of a sample pad is not present in an observation zone and swells into the observation zone, notifying an operator that the sample pad has absorbed a sufficient amount of fluid. In certain implementations, a portion of the handle can deform when a sample pad swells, notifying an operator that the sample pad has absorbed a sufficient amount of fluid.

In certain implementations, a sealing member is disposed on or in a handle and/or a stem of a sample collection device. Sometimes a sample collection device includes a first sealing member and a second sealing member each disposed on the stem. Any suitable sealing member can be incorporated on or in a sample collection device, including without limitation one or more o-rings constructed from a deformable material (e.g., an elastomer).

A sample collection device can include a connector member configured to engage with a connector member counterpart disposed on or in a test device. A connector member is a lock member in certain implementations. A connector member can be disposed on or in any suitable location of the sample collection device for connecting engagement with a connector member counterpart disposed on or in a test device described herein. A connector member and/or lock member can be reversible or irreversible under a tool-less pulling force generated by a human of average strength.

A lock member sometimes is disposed on or in the stem and sometimes is disposed on or in the handle of the sample collection device. In certain instances, a lock member is disposed on the stem between the handle and the sealing member. Any suitable lock member can be incorporated on or in a sample collection device, non-limiting examples of which include an interference-fit member (e.g., projection/receptacle (e.g., annular projection/annular groove)) or a threaded member (e.g., threaded grooves/threaded projections). In certain instances, a handle of a sample collection device includes an interior surface spaced from an exterior surface of the stem, and the interior surface spaced from the exterior surface of the stem includes a threaded lock member. In certain implementations, a stem of a sample collection device includes an annular projection disposed between the stem proximal terminus and a sealing member (e.g., o-ring).

In certain implementations, (i) a handle of a sample collection device is a tubular handle or includes a tubular portion, that includes a handle proximal terminus, a handle distal terminus and a handle wall between the handle proximal terminus and the handle distal terminus; and/or (ii) a stem is a tubular stem or includes a tubular portion, that includes a stem proximal terminus, a stem distal terminus, a stem wall between the stem proximal terminus and the stem distal terminus and a tubular stem interior; where the stem proximal terminus is in connection with the handle distal terminus. In instances where the stem is a tubular stem or includes a tubular portion, the sample pad can be a cylindrical pad or include a cylindrical portion. In certain implementations, a first portion of the sample pad side is inserted within the tubular stem interior (i.e., a proximal portion of the sample pad), and a second portion of the sample pad side extends from the stem distal terminus (i.e., a distal portion of the sample pad).

The sample pad of a sample collection device can include sample fluid. The sample fluid generally is absorbed in the sample pad and can be any suitable fluid for analysis in a device described herein. A sample fluid sometimes is a biological sample fluid that contains molecules from a biological entity. A sample fluid can be from any suitable biological entity, non-limiting examples of which include a unicellular organism, multi-cellular organism, a cell from a multi-cellular organism, eukaryotic cell, prokaryotic cell, microorganism, bacterium, archaeon, fungus, plant, virus, organelle (e.g., mitochondria or chloroplast), liposomal vector and extracellular vesicle. A sample fluid sometimes is from a human subject or non-human subject. A non-human subject sometimes is a mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an embryo, a fetus, infant, child, adult). Non-limiting examples of a biological fluid from a human or non-human subject include saliva, urine, blood (e.g., whole blood, blood fraction), semen and vaginal fluid, and can be derived from a biological sample (e.g., fecal matter, ear wax, biopsy sample). A sample fluid sometimes includes molecules from an environmental source, non-limiting examples of which include a water sample, soil sample, or other environmental sample that may contain a toxic substance (e.g., bioharzard substance).

A sample fluid absorbed into a sample pad can be an unprocessed sample fluid directly from a source (e.g., a subject) that optionally has been stored, or can be a fluid that has been processed and optionally stored (e.g., diluted, concentrated, treated with one or more reagents (e.g., a preservative)). A sample fluid can be absorbed into a sample pad in any suitable manner, including for example, directly contacting the sample pad with a sample fluid source. For example, a sample pad may be placed on or in the body of a subject (e.g., a sample pad may be placed in the mouth of a subject for absorption of a saliva sample). In certain implementations, a sample can be collected in a container and then a sample pad can be contacted with a collected sample fluid, or a fluid containing a collected sample, for absorption into the sample pad (e.g., collecting saliva in a container and then absorbing the collected saliva into a sample pad).

A sample collection device can be joined with a test device after absorbing a sample fluid in the sample pad without processing or storing of the sample fluid. A sample collection device can be joined with a test device after the sample collection device has been stored (e.g., in a sealed container, e.g., in a sealed bag) and/or optionally processed, or after the sample has been collected in a container and/or optionally processed.

A particular non-limiting implementation of a sample collection device component is device 200 illustrated in FIG. 1 to FIG. 3G, which also is referred to herein as a “sample collector.” As shown in FIG. 1, sample collection device 200 is configured for insertion into test device 100, and is configured for sealing attachment and locked attachment to test device 100. Sample collection device 200 is configured for lateral insertion into test device 100, with insertion in a direction parallel to lateral axis 198, and in a proximal to distal direction, as shown in FIG. 1 and FIG. 10, for example.

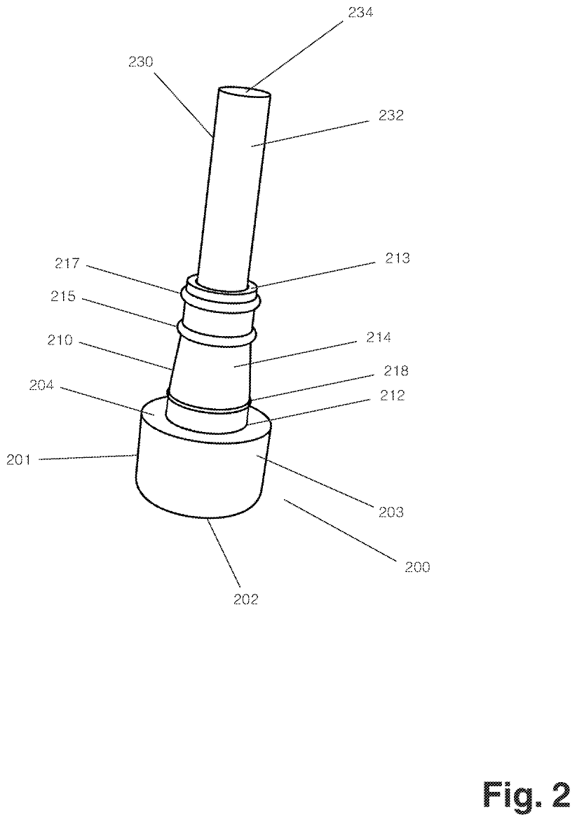

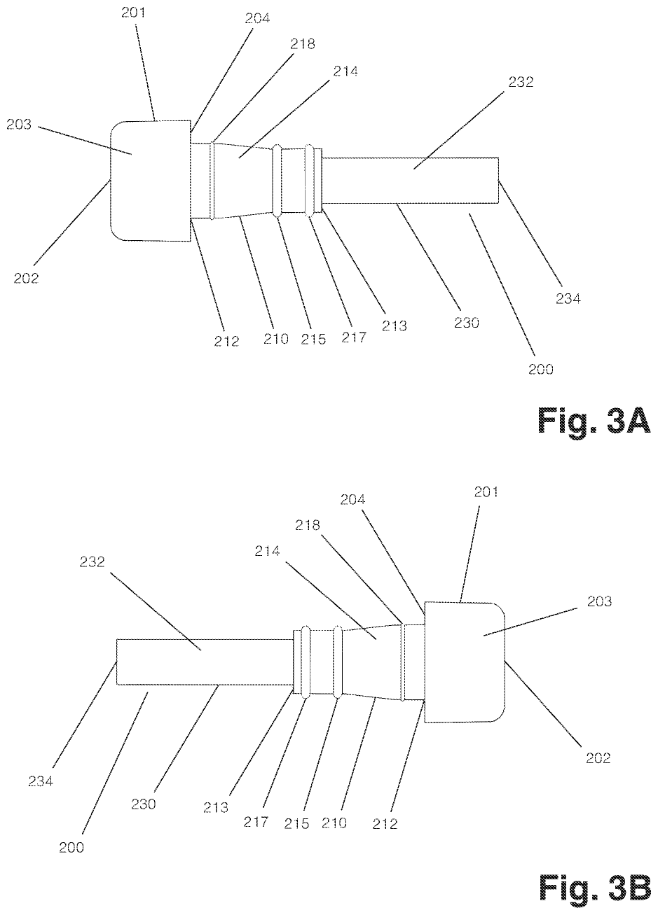

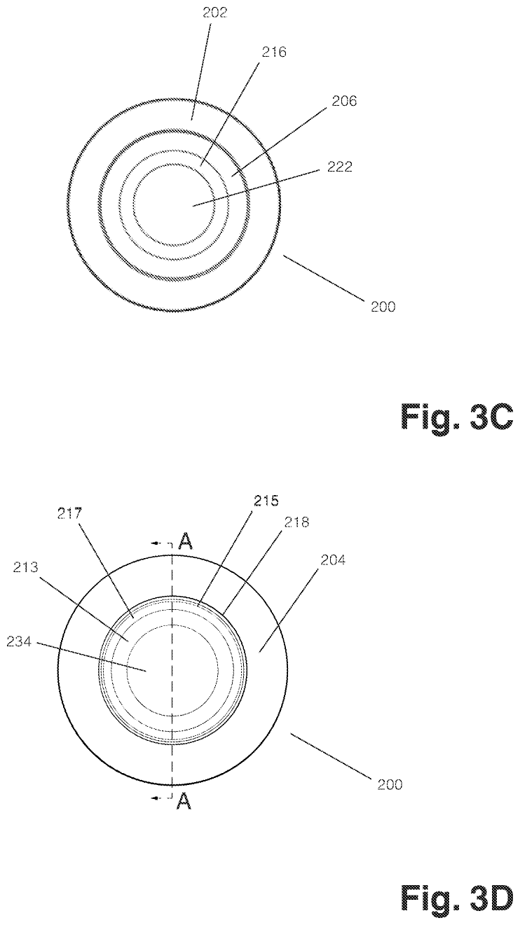

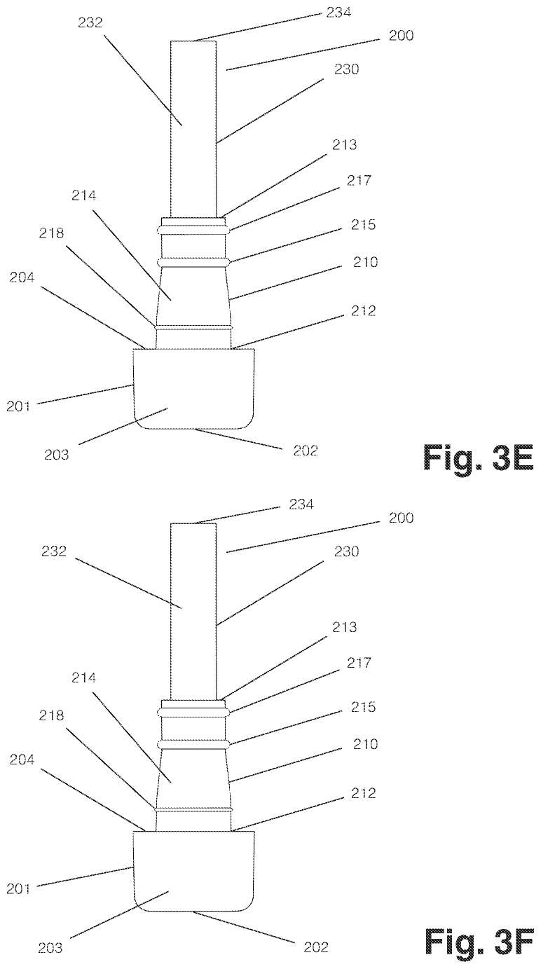

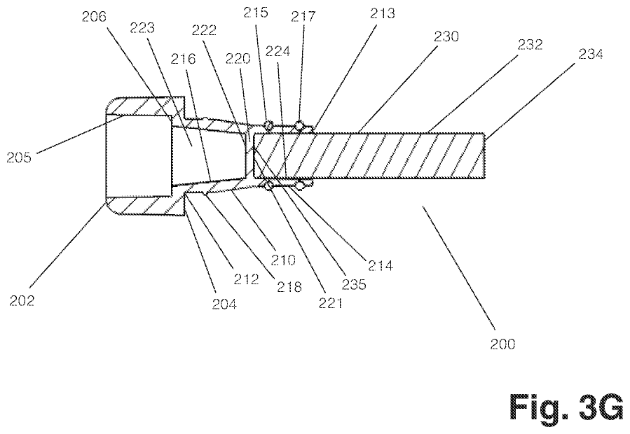

Sample collection device 200 includes a cylindrical handle 201 having a handle proximal terminus 202, a handle distal terminus 204, and a handle sidewall 203 between the proximal terminus 202 and the distal terminus 204. Sample collection device handle 201 includes a hollow portion, and sidewall 203 includes an exterior surface (as shown in FIG. 2) and an interior surface 205 (as shown in FIG. 3G). Handle member 201 of sample collection device 200 includes an interior step 206 disposed at the distal terminus of the interior of the handle, which transitions from the handle portion to the stem portion, the latter of which is described hereafter.

Sample collection device 200 includes a tubular stem portion 210, having a proximal terminus 212, distal terminus 213, and sidewall 214, as shown in FIG. 2. Stem member 210 is a frustum, with sidewall 214 tapering from proximal terminus 212 to distal terminus 213, where the internal diameter at proximal terminus 212 is greater than the internal diameter at distal terminus 213. Stem member 210 of sample collection device 200 is hollow and stem sidewall 214 includes an exterior surface (as shown in FIG. 2), and an interior surface (e.g., interior surface 216 and 224 shown in FIG. 3G). In sample collection device 200, stem 210 includes transverse interior wall 220 that spans the stem interior and having a major dimension parallel to longitudinal axis 199 shown in FIG. 10, where sample collection device 200 has the same orientation relative to test device 100 as shown in FIG. 1. Interior wall 220, having proximal surface 222 and distal surface 221, separates the interior of stem 210 into (i) a first stem interior compartment in the stem having lumen 223 and a proximal terminus adjacent to the interior of handle member 201, and (ii) a second stem interior compartment, distally disposed with respect to the first interior compartment, that includes stem sidewall interior surface 224, as shown in FIG. 3G.

Stem member 210 includes two o-ring sealing members 215 and 217 each disposed within an annular groove receptacle located on the exterior surface of stem sidewall 214, opposite the second stem interior compartment. A single o-ring sealing member or one or more other types of sealing members can replace o-ring sealing members 215 and 217 of stem member 210 in certain implementations. A stem member sometimes includes no sealing members in certain instances. Stem member 210 also includes stem annular protrusion 218, disposed between (i) the handle member distal terminus 204, and (ii) sealing member 215 and stem interior wall 220. Annular protrusion 218 is a connection member configured to engage with and connect to a connector member counterpart in test device 100, i.e., annular groove 178b bounded by recessed surface 122 and recessed surface 132 of test device housing 101 and recessed edge 178a in cartridge housing 170. Connector member annular protrusion 218 is a lock member and also is referred to herein as a click lock feature.

Sample collection device 200 also includes cylindrical sample pad 230, which also is referred to as an absorbent collection pad, which includes pad side surface 232, pad distal terminus (distal surface) 234, and pad proximal terminus (proximal surface) 235, as depicted in FIG. 2 and FIG. 3G. Sample pad 230 has a lateral length 290a, which is the major dimension of the sample pad and is parallel to lateral axis 198 (see FIG. 10), when the sample collection device 200 is in the same orientation relative to test device 100 as depicted in FIG. 1. A proximally-disposed portion of sample pad 230 is disposed within the second stem interior compartment, with sample pad proximal terminus 235 contacting distal surface 221 of stem transverse interior wall 220. A portion of sample pad side surface 232 of the proximally-disposed portion contacts interior stem sidewall surface 224 within the second stem interior compartment. A distally-disposed portion of sample pad 230 extends from stem member 210, past stem distal terminus 213. The portion of sample pad side 232 in this distally-disposed portion can contact a fluid sample for absorption of sample fluid into sample pad 230, and can be disposed within cartridge housing lumen 173d and 173e when sample collection device 200 is joined to test device 100 (e.g., sealingly joined and/or in locked engagement). Distal surface 234 of sample pad 230 also can contact a fluid sample for absorption of sample fluid into sample pad 230, and when sample collection device 200 is joined to test device 100, can (i) contact cartridge housing terminal wall interior surface 175b when there is no cartridge housing pad disposed in the interior of cartridge housing 170, (ii) contact a proximal surface of a cartridge housing pad (e.g., proximal surface 145a of cartridge housing pad 145) when such a pad is disposed in the interior of cartridge housing 170.

As illustrated in FIG. 3C, FIG. 3D and FIG. 3G, cylindrical handle member 201, tubular stem member 210 and cylindrical sample pad 230 are concentrically disposed. Stated another way, (i) the midpoint between the sidewall exterior surfaces at proximal terminus 202 of handle 201, (ii) the midpoint between the sidewall exterior surfaces at proximal terminus 212 of stem 210, and (iii) the midpoint between the sidewall exterior surfaces at proximal surface 235 of sample pad 230, are concentric.

An observation zone as described herein can be located in stem portion 210, sometimes between sealing rings 215 and 217, and/or sometimes at or adjacent to stem transverse interior wall 220. In certain implementations, a button, connected to the stem transverse interior wall 220 and proximally spaced from wall 220, can be displaced by swelling-induced displacement by the sample pad, and displacement of the button can be detected by an operator (e.g., by touch). An operator can observe a change in the sample pad upon a sufficient amount of absorbed sample fluid by the sample pad in the first stem interior compartment and/or the second stem interior compartment, for example. In an implementation for which a sample pad swells when contacted with a fluid, sample pad proximal terminus 235 sometimes is spaced from the stem transverse interior wall 220 and the sample pad deforms closer to or in contact with the transverse interior wall 220 when the sample pad swells when contacted with a fluid.

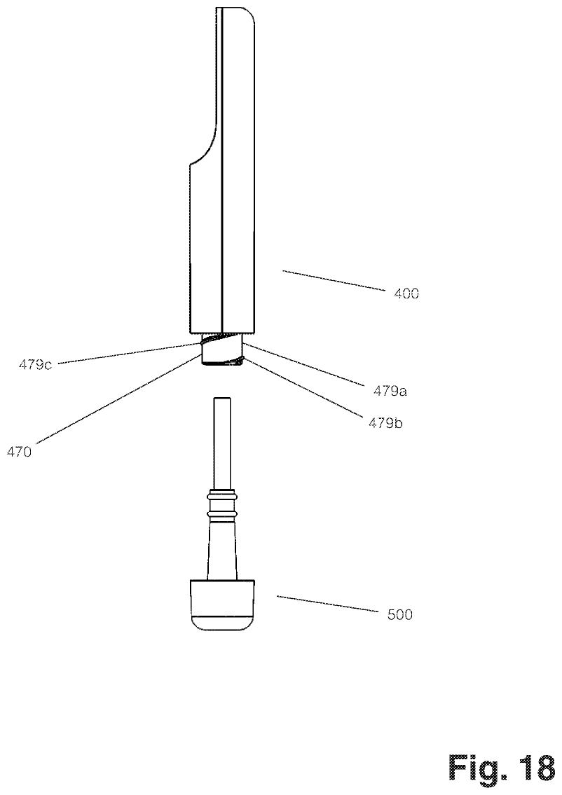

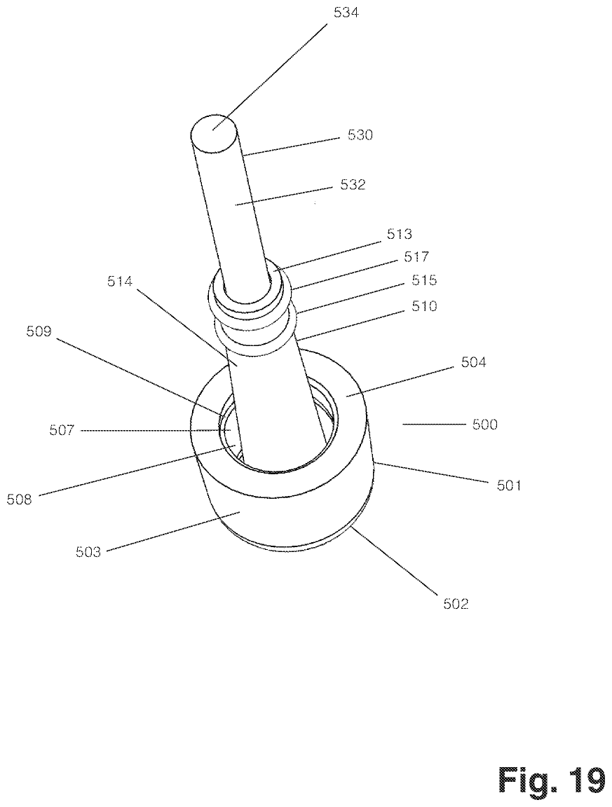

A non-limiting sample collection device that connects to a test device via a threaded member (i.e., a screw thread) is depicted as sample collection device 500, which is illustrated in FIG. 18 and FIG. 19. Sample collection device 500 includes sample pad 530 (also referred to as an absorbent collection pad), sample pad side surface 532 and sample pad distal terminus (distal surface) 534. Sample collection device 500 includes handle member 501, handle proximal terminus 502, handle distal terminus 504, and handle sidewall 503 between proximal terminus 502 and distal terminus 504. Handle sidewall 503 includes an exterior surface (i.e., shown as element 503 in FIG. 19). Sample collection device 500 also includes stem member 510, which includes stem distal terminus 513, stem sidewall 514 (exterior surface illustrated in FIG. 19) and sealing members 515 and 517 disposed on stem sidewall 514. Surrounding a proximal portion of stem member 510 is handle interior surface 507, which is spaced from and opposes the exterior surface of stem sidewall 514 in the proximal portion of stem member 510. Handle interior surface 507 is separated by void 508 from stem sidewall 514. A connector member counterpart thread 509 (i.e., a threaded projection) is disposed on the handle interior surface 507 within void 508. Connector member counterpart 509 can be a lock member counterpart configured to engage a thread disposed on or in test device 400. In certain implementations, threaded projection 509 is replaced by a threaded groove, and sometimes in place of thread 509 a threaded projection or threaded groove is disposed on stem sidewall 514 instead of handle interior surface 507.

### Test Device Component

Provided in certain implementations is a test device component that includes: a hollow test device housing that includes a proximal terminus, a distal terminus, a sidewall between the proximal terminus and distal terminus, an interior, and a window disposed in the sidewall; a cartridge disposed within the test device housing, the cartridge including: a hollow cartridge housing that includes a proximal terminus distally disposed from, and adjacent to, the test device housing proximal terminus, a distal terminus, a sidewall disposed between the proximal terminus and the distal terminus, an interior, and a terminal wall disposed at the distal terminus, and a hollow terminal projection member extending from the terminal wall of the cartridge housing, the terminal projection member including a proximal terminus disposed on the terminal wall, a distal terminus opposite the terminal wall, a sidewall disposed between the proximal terminus and distal terminus, and an interior; a porous transfer pad disposed within the terminal projection member of the cartridge; and a lateral flow strip disposed in the test device housing adjacent to the transfer pad and distally disposed from the cartridge, the strip comprising a proximal surface, a distal surface, and an anterior surface and an opposite posterior surface each disposed between the proximal surface and the distal surface; where the window in the test device housing opposes a portion of the strip anterior side. The term “distally disposed from” is relative to lateral axis 198 that is shown in FIG. 10 in the proximal to distal direction. The terms “anterior” and “posterior” are relative to longitudinal axis 199, which is shown in FIG. 10 in the posterior to anterior direction. In certain implementations, a sidewall of a test device housing, cartridge housing and cartridge terminal projection each independently is one continuous sidewall (i.e., in the case of a cylindrical or frustum portion of the test device) and sometimes is two, three, four or more sidewalls (e.g., in the case of a rectilinear portion of the test device).

The test device housing, cartridge housing and cartridge terminal projection can be of any suitable configuration for (i) receiving a sample pad or portion thereof from a sample collection device through a proximal opening of the test device, or (ii) receiving in the interior of the cartridge housing lumen the sample pad or portion thereof, or (iii) laterally compressing the sample pad with the distal surface of the transfer pad contacting the terminal wall of the cartridge housing or a cartridge housing pad directly contacting the terminal wall or in a series of cartridge housing pads in which one cartridge housing pad contacts the terminal wall, or (iv) transmitting sample fluid expressed by the sample pad through the terminal wall to the transfer pad and to the lateral flow strip, or (v) a combination of two, three or all of (i), (ii), (iii) and (iv).

In certain implementations, (i) the test device housing is a tube, or (ii) the cartridge housing is a tube, or (iii) the cartridge terminal projection is a tube, or (iv) a combination of two or more of (i), (ii) and (iii). In certain instances, the interior surface of the sidewall of each of the test device housing, cartridge housing, and cartridge terminal projection defines an interior lumen. In certain implementations, (i) the midpoint between sidewall interior surfaces at the proximal terminus of the cartridge housing, (ii) the midpoint between the sidewall interior surfaces at the proximal terminus of the test device housing, and/or (iii) the midpoint between the sidewall interior surface at the proximal terminus of the cartridge terminal extension, are concentric.

The test device housing and the cartridge sometimes independently include polypropylene, polyethylene, acrylonitrile butadiene styrene, or a combination of two or more of the foregoing. In certain implementations, the test device housing is a two-piece housing comprising an anterior member and a posterior member, and sometimes the posterior member includes at least one connector and the anterior member includes at least one connector counterpart. In certain instances, the cartridge is a unitary member. A cartridge and a test device housing can be separate entities (e.g., separate molded entities) that are assembled into a test device component, or can be incorporated together in a unitary test device component (e.g., the cartridge and test device housing are molded as part of one molded component). In certain implementations, a cartridge contains a posterior member and an anterior member, the anterior member of the cartridge is co-molded with an anterior test device housing member and the posterior member of the cartridge is co-molded with a posterior test device housing member.

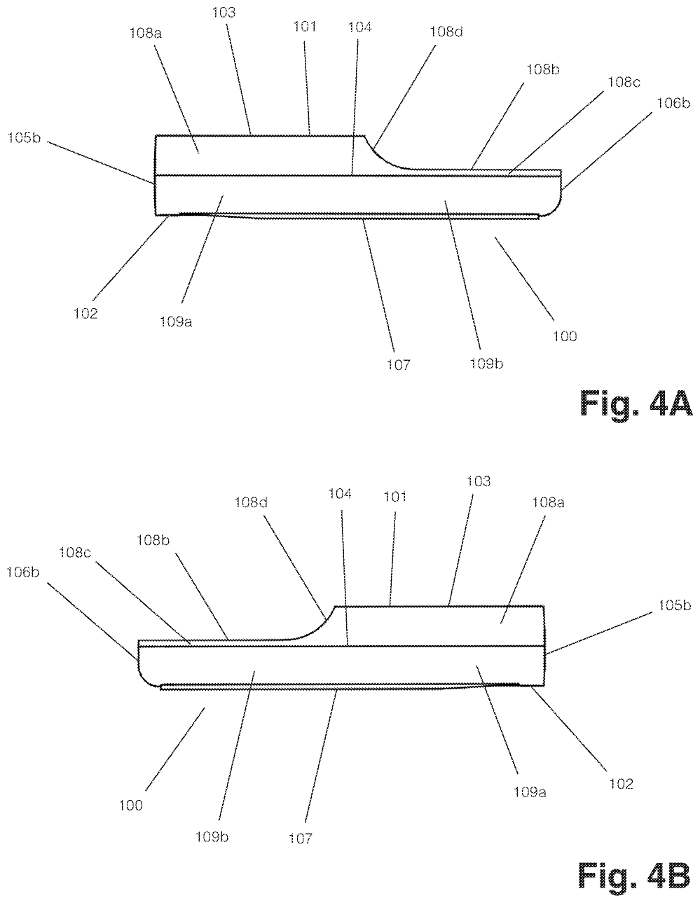

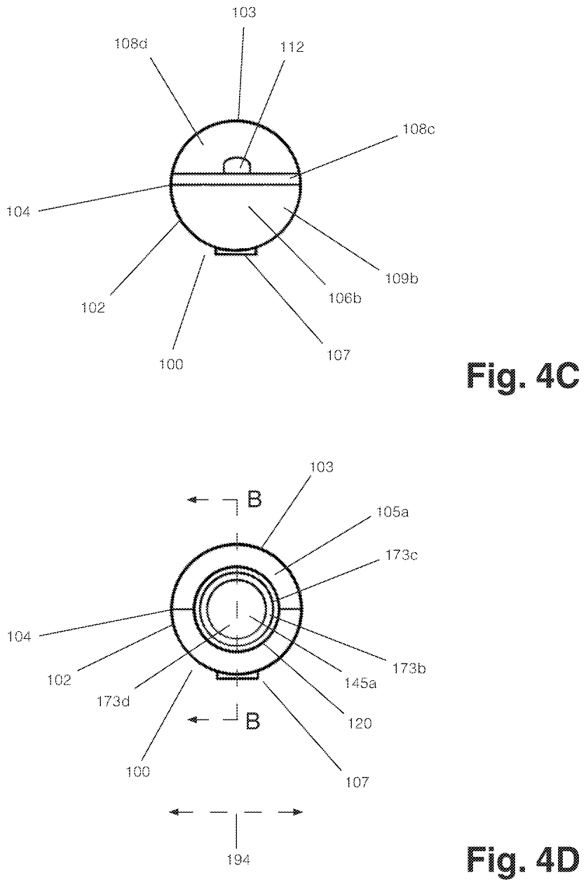

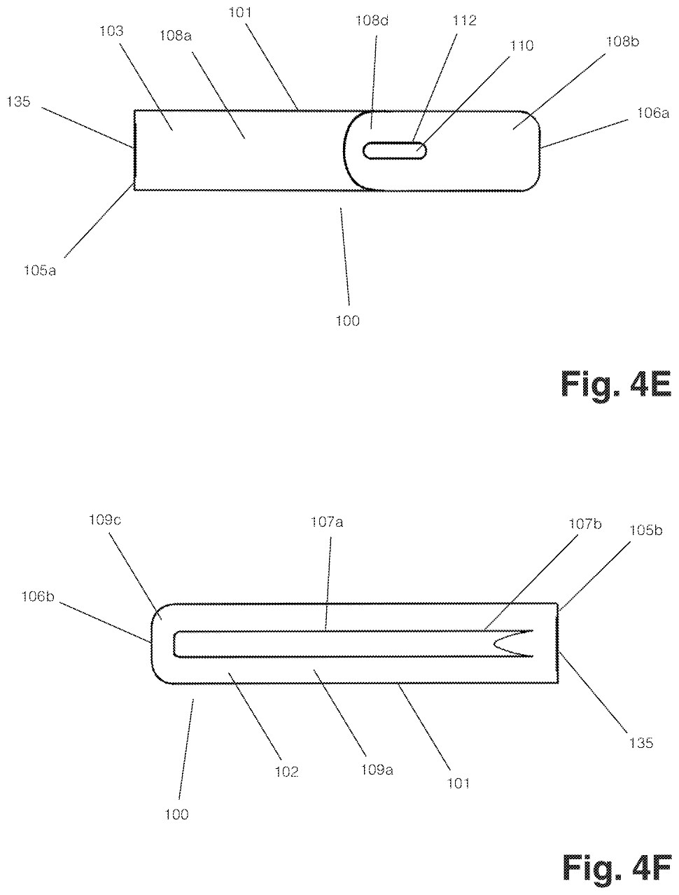

In certain implementations, the test device housing includes a first region. The first region of the test device housing sometimes is a tube that includes a circular or ovoid cross section, and sometimes the first region of the test device housing is a cylindrical tube. The first region of the test device housing often is co-terminal with the proximal terminus of the test device housing. In certain instances, the test device housing includes a second region. A second region of the test device housing sometimes is a tube that includes a lateral planar cylindrical segment region, where the second region often is co-terminal with the distal terminus of the test device housing. In certain implementations, a test device housing includes a third region. The third region of the test device housing sometimes is a tube that includes a lateral arcoid cylindrical segment region. Curvature of the lateral arcoid cylindrical region generally is defined by an edge (e.g., a linear edge) of a virtual plane rotated around a transversely-aligned axis spaced from the anterior surface of the test device housing, where the transversely aligned axis is parallel to the transverse axis 194 illustrated in FIG. 4D. A third region often is disposed between the first region and the second region of the test device housing, and is co-extensive with the first region and the second region of the test device housing.

In certain implementations, the test device housing includes a bore and the perimeter of the bore defines the window. A window often is an opening defined by a bore in a test device housing, at which the test strip within the housing is exposed. Sometimes a window includes a transparent or translucent lens through which an optical signal from the lateral flow strip can transmit. The perimeter of the bore sometimes is ovoid or polygonal or a combination thereof. The perimeter of the bore sometimes is a combination of ovoid boundaries (e.g., ovoid proximal and distal boundaries) and linear laterally-disposed boundaries. In certain instances, the window is disposed in a portion of the second region and an adjoining portion of the third region of the test device housing.

A cartridge housing can be of any suitable geometry that (i) contains a sample pad or portion thereof of a sample collection device component that can be joined with a test device, or (ii) orients the terminal wall of the cartridge housing in a position that facilitates compression of the sample pad, with or without a cartridge housing pad disposed between the terminal wall and the sample pad distal terminus, or (iii) a combination of (i) and (ii). In certain implementations, the cartridge housing is a tube that includes a circular or ovoid cross section. The sidewall of the cartridge housing or portion thereof sometimes tapers from the proximal terminus towards the distal terminus, where the interior diameter at the proximal terminus is greater than the interior diameter of a portion distally-spaced from the proximal terminus. In certain instances, the cartridge housing includes a frustum portion, and sometimes includes two frustum portions, with a proximally-disposed first frustum portion defined by sidewalls having one exterior draft angle, and another distally-disposed second frustum portion contiguous with the first frustum portion and defined by sidewalls having a second exterior draft angle, where the second draft angle is greater than the first draft angle.

In certain implementations, the cartridge terminal projection is a tube configured to retain a transfer pad or portion thereof. A cartridge terminal projection sometimes includes a polygon cross section, polygon distal terminus and/or polygon proximal terminus. The polygon can be a quadrilateral, rectangle or square in certain instances.

A transfer pad can be disposed in a cartridge terminal projection member in any suitable manner that permits transmission of fluid from the sample pad to the lateral flow strip. In certain implementations, a proximal portion of the transfer pad is disposed within the cartridge terminal projection member and a distal portion of the transfer pad extends from the distal terminus of the cartridge terminal projection member. In certain instances, the cartridge housing sidewall includes an interior surface and the cartridge housing terminal wall includes an interior surface having a perimeter coextensive with the cartridge sidewall interior surface. In certain implementations, a transfer pad includes an anterior surface and an opposite posterior surface each between the proximal surface and the distal surface of the pad, and the anterior side surface and the posterior side surface of the transfer pad each contact an interior surface of the cartridge terminal projection member. A transfer pad can be joined to a cartridge terminal projection member in any suitable manner, and sometimes a transfer pad is disposed within the cartridge terminal projection member by an interference fit or sometimes is adhered to a terminal projection member by a weld (e.g., adhesive or sonic weld).

In certain implementations, the cartridge terminal wall includes one or more bores. A cartridge terminal projection member often includes an interior and often the one or more bores in the cartridge terminal wall each include a distally disposed aperture within the interior of the terminal projection member. In certain instances, a transfer pad includes a proximal surface and a distal surface and the proximal surface of the transfer pad contacts an exterior surface of the cartridge terminal wall and the one or more bores in the terminal wall. The surface area of a bore (where there is one bore) or combined surface area of bores (where there are multiple bores) sometimes is about 1 square millimeter to about 50 square millimeters, or about 5 square millimeters to about 20 square millimeters, or about 10 square millimeters to about 15 square millimeters, or about 1, 2, 3, 4, 5, 6, 7, 8, 9, 10, 11, 12, 13, 14, 15, 16, 17, 18, 19, 20, 25, 30, 35, 40, 45 or 50 square millimeters. The surface area of the interior surface of the terminal wall of the cartridge housing, inclusive of the surface area of one or more bores in the terminal wall, sometimes is about 20 square millimeters to about 100 square millimeters, or about 40 square millimeters to about 80 square millimeters, or about 50 square millimeters to about 70 square millimeters, or about 20, 25, 30, 35, 40, 45, 50, 55, 56, 57, 58, 59, 60, 61, 62, 63, 64, 65, 66, 67, 68, 69, 70, 71, 72, 73, 74, 75, 80, 85, 90, 95 or 100 square millimeters. A ratio of (i) the surface area of the bore or combined surface area of multiple bores, to (ii) the surface area of the interior surface of the terminal wall of the cartridge housing (inclusive of the surface area of one or more bores in the terminal wall), is about 0.05 to about 2, or about 0.08 to about 0.5, or about 0.1 to about 0.3, or about 0.15 to about 0.30, or about 0.20 to about 0.25, or about 0.05, 0.07, 0.09, 0.10, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.40, 0.45 or 0.50.

A transfer pad can be disposed relative to a lateral flow strip in any suitable orientation for transmission of sample fluid from the transfer pad to the lateral flow strip. In certain implementations, a portion of the posterior side surface of the transfer pad contacts a portion of the anterior surface of the lateral flow strip. In certain instances, a portion of the distal surface of the transfer pad contacts a portion of the proximal surface of the strip. In certain implementations, the lateral flow strip includes a lateral length and a perpendicular transverse width, the transfer pad includes a lateral length and a perpendicular transverse width, and the transfer pad width is substantially the same or the same as the lateral flow strip width. A transverse width is parallel to transverse axis 194 illustrated in FIG. 4D and a lateral length is parallel to lateral axis 198 illustrated in FIG. 10.

A lateral flow strip can be contained in a test device in any suitable manner that permits fluid transmission from the transfer pad to the lateral flow strip and observation of an anterior portion of the strip through a window in the test device housing. In certain implementations, a lateral flow strip is retained in the test device housing by one or more strip retaining members disposed on an interior surface of the test device housing sidewall. In certain instances, each of the one or more strip retaining members extends from a posterior sidewall interior surface of the test device housing sidewall. The one or more strip retaining members sometimes include (i) one or more laterally disposed ribs, or (ii) one or more containment posts, or (iii) a combination of (i) and (ii). The lateral length of a laterally disposed rib generally is the major dimension of such a rib, and the lateral length is parallel to lateral axis 198 illustrated in FIG. 10. For instances in which a test device includes one or more laterally disposed ribs, the posterior surface of the strip or portion thereof sometimes contacts an anterior edge of each of the one or more laterally disposed ribs of the test device housing. A lateral flow strip generally includes a laterally disposed edge on each side of the posterior surface of the strip, and for instances in which a test device includes one or more containment posts, each laterally disposed edge of the strip sometimes contacts a portion of one or more containment posts disposed in the test device housing. A longitudinal length often is the major dimension of a containment post, where the longitudinal length is parallel to longitudinal axis 199 illustrated in FIG. 10. A containment post can include any suitable minor dimension profile for retaining a lateral flow strip at a contact point with the edge of the strip, and sometimes a containment post minor dimension profile is a polygon (e.g., triangle or quadrilateral (e.g., square, rectangle, rhombus)) or is ovoid (e.g., oval or circle).

A test device can include one or more members that facilitate fluid transfer from a transfer pad to a lateral flow strip. In certain implementations, the test device housing includes an anterior sidewall interior surface and an extension bar extending from the anterior sidewall interior surface. An extension bar sometimes includes a posterior surface (i.e., the term posterior surface is used interchangeably with the term posterior edge for an extension bar). The posterior edge of the extension bar, or portion of the posterior edge, often contacts a portion of the anterior surface of the transfer pad, and sometimes contacts a portion of the anterior surface of the transfer pad opposite the anterior surface of the lateral flow strip. An anterior sidewall interior surface refers to an interior surface disposed in an anterior portion of the test device housing sidewall, and a posterior sidewall interior surface refers to an interior surface disposed in a posterior portion of the test device housing sidewall (see, e.g., longitudinal axis 199 disposed in the posterior to anterior direction). Without being limited by theory, an extension bar can apply downward pressure (i.e., in the anterior to posterior direction) to the transfer pad and to the test strip, which can partially restrict or reduce fluid flow from the transfer pad to the test strip compared to implementations in which an extension bar does not apply pressure to the transfer pad. Reduction of fluid flow by a transfer pad can reduce potential for flooding of the test strip as sample fluid is transmitted from the transfer pad, and pressure exerted by the extension bar can assure contact of the transfer pad uniformly along the interface of the transfer pad and the test strip.

A test device can include one or more members that retain the cartridge within the test device housing interior and/or facilitate fluid transmission from the sample pad to the lateral flow strip. In certain implementations, a test device includes (i) a first extension wall disposed on and extending from a posterior sidewall interior surface of the test device housing sidewall, and/or (ii) a second extension wall disposed on and extending from an anteriorly-disposed interior surface of the test device housing sidewall. For implementations in which the test device includes the first extension wall and the second extension wall, the first extension wall can include an anterior edge and the second extension wall can include a posterior edge, where the anterior edge of the first extension wall sometimes opposes and sometimes is spaced from the posterior edge of the second extension wall. In certain implementations, the cartridge terminal projection includes an anterior wall and a posterior wall, the anterior edge of the first extension wall contacts an exterior surface of the terminal projection posterior wall, and the posterior edge of the second extension wall contacts an exterior surface of the terminal projection anterior wall.

In certain instances, a test device includes a third extension wall and a fourth extension wall each disposed on and extending from a posteriorly-disposed sidewall interior surface of the test device housing sidewall. The third extension wall sometimes includes an interior side (i.e., a face of the extension wall opposed to the center of the test device housing) that opposes an interior side of the fourth lateral extension wall. Sometimes a portion of the interior side of the third extension wall contacts a side of the first extension wall and a portion of the interior side of the fourth extension wall contacts an opposing side of the first extension wall. In certain implementations, the transverse length of the first extension wall is the major dimension of the first extension wall (i.e., the major dimension is parallel to transverse axis 194 shown in FIG. 4D), the third extension wall and the fourth extension wall each include a lateral length that is the major dimension of each wall (i.e., the major dimension is parallel to lateral axis 198), and the first extension wall is perpendicularly disposed to each of the third extension wall and the fourth extension wall. Stated another way, the first extension wall sometimes has a major dimension (i.e., length) aligned parallel to transverse axis 194 (shown in FIG. 4D)) and perpendicular to lateral axis 198 (shown in FIG. 10), and the third and fourth extension walls sometimes each have a major dimension (i.e., length) aligned parallel to lateral axis 198 and perpendicular to the transverse axis 194.

In certain implementations, a cartridge terminal projection includes a sidewall and an opposing sidewall between the anterior wall and the posterior wall, a portion of the internal side of the third extension wall contacts an exterior surface of the cartridge terminal projection sidewall, and a portion of the internal side of the fourth extension wall, contacts an exterior surface of the cartridge terminal projection opposing sidewall in a test device. By such contacts, each of the first, second, third and fourth extension walls in part can retain the cartridge terminal projection, and thereby in part retain the cartridge, in the test device housing. By such contacts, the first and second extension walls can restrain longitudinal movement of the cartridge, and the third and fourth extension walls can restrain transverse movement of the cartridge, within the test device housing.

A test device can include additional members that retain the cartridge within the test device housing interior. In certain implementations, the test device housing includes a proximal opening rim and an annular recessed surface distally disposed and adjacent to the proximal opening rim, where the proximal terminus of the cartridge housing contacts the annular recessed surface of the test device housing. In certain instances, the test device housing includes an annular projection disposed on an interior surface of the test device housing sidewall and distally disposed from the proximal opening rim, where the annular projection contacts a portion of an exterior surface of the cartridge housing sidewall. In certain implementations, the cartridge housing includes an annular collar extending from an exterior surface of the cartridge housing sidewall, the test device housing includes a proximal opening rim and an annular recessed surface distally disposed and adjacent to the proximal opening rim, and the collar contacts the annular recessed surface of the rim of the test device housing. In certain implementations, the test device housing includes an annular projection disposed on an interior surface of the test device housing sidewall and distally disposed from the proximal opening rim, and a surface of the collar contacts the annular projection. In certain instances, surfaces of the collar contact the annular recessed surface of the rim, a proximally-disposed surface of the annular projection, and a portion of the interior surface of the test device housing sidewall disposed between the annular recessed surface and the annular projection. By such contacts, the annular projection and recessed surface of the rim of the test device housing, and collar of the cartridge, when present, can retain the cartridge in the test device by restraining longitudinal movement and/or transverse movement and/or lateral movement of the cartridge in the test device housing.

A test device can include further members that retain the cartridge within the test device housing interior and/or facilitate fluid transmission from the sample pad to the lateral flow strip. In certain implementations, a test device can include post members each disposed on and extending from a posterior sidewall interior surface of the test device housing sidewall. A portion of one post member can contact a portion of the distal terminus of the terminal projection of the cartridge. A portion of another post member can contact another portion of the distal terminus of the terminal projection of the cartridge. In certain instances, the third extension wall and the fourth extension wall of the test device housing each include a distal side, where one post member is integrated with the distal side of the third extension wall, and another post member is integrated with the distal side of the fourth extension wall. In certain implementations, the third extension wall and the fourth extension wall each include a proximal side, where the proximal side of the third extension wall contacts an exterior portion of the terminal wall of the cartridge housing, and the proximal side of the fourth extension wall contacts another exterior portion of the terminal wall of the cartridge housing. By such contacts, (i) the post members, or (ii) the annular recessed surface adjacent to the proximal opening rim, or (iii) proximal side of the third extension wall and the fourth extension wall, or (iv) combination of two or more of (i), (ii) and (iii), in part can retain the cartridge in the test device housing by restraining lateral movement of the cartridge.

A test device can include a connector member configured to connect to a connector member counterpart disposed on or in a sample collection device component. A connector member sometimes is a lock member that locks the test device to a sample collection device component. Any suitable type of connector member can be present on or in a test device and in of any suitable location. A connector member sometimes is a groove or protrusion (e.g., an annular groove or annular protrusion), which can engage with a counterpart member by an interference fit, and sometimes is a threaded connector (e.g., a threaded groove or a threaded projection). A connector member/lock member can be disposed on an interior surface of the test device housing or the cartridge housing, or on an exterior surface of the test device housing (e.g., on or in the proximal edge of the housing) or the cartridge housing. In certain instances, the cartridge housing includes an annular recessed edge adjacent to the proximal terminus, the test device housing includes a proximal opening rim and an annular recessed surface adjacent to the proximal opening rim, and the annular groove is disposed between the annular recessed edge of the cartridge housing and the annular recessed surface of the test device housing. In certain instances, the cartridge housing includes a hollow shank extending from the cartridge housing proximal terminus and the lock member counterpart is a threaded member disposed on the shank. The shank sometimes is cylindrical and sometimes includes a wall portion having an exterior surface and interior surface. Threads can be disposed on the shank exterior surface (as illustrated in FIG. 18) or on the shank interior surface and can be threaded grooves or threaded projections.

A test device can include a sealing member configured for sealing connection of the sample collection device component to the test device component. A sealing member counterpart can be disposed on an interior surface of the test device housing and/or an interior surface of the cartridge housing. A sealing member counterpart in the test device is optional, and a test device component can seal to a sample collection component without a sealing member counterpart in the test device, in certain implementations. A sealing member sometimes is not present within the test device (e.g., not present on or in an interior wall portion of the test device housing or the cartridge housing).

A test device component can include one more porous cartridge pads disposed in an interior portion of the cartridge housing, in certain implementations. In certain instances, each of the one or more porous cartridge pads includes a proximal surface, a distal surface and a side surface disposed between the proximal surface and the distal surface. The terminal wall of the cartridge housing typically includes an interior surface, and the distal surface of a cartridge pad can contact the interior surface of the cartridge housing terminal wall. A test device sometimes includes two or more porous cartridge pads disposed in a stacked assembly. In a stacked assembly a first pad often is proximally disposed relative to an adjacent second pad, the first pad and the second pad each generally include a proximal surface, a distal surface and a side surface disposed between the proximal surface and the distal surface, and the distal surface of the first pad often contacts the proximal surface of the second pad. In certain implementations, the transfer pad is in fluid communication with the one or more porous cartridge pads, and sometimes the proximal surface of the transfer pad is in fluid communication, via the bores in the terminal wall of the cartridge housing, with the distal surface of a cartridge housing pad. In certain instances, the cartridge housing sidewall includes an interior surface, and the side surface of each of the one or more cartridge pads contacts a portion of the interior surface of the cartridge housing sidewall. A test device sometimes includes no cartridge pad disposed in an interior portion of the cartridge housing.

In certain implementations, the transfer pad of a test device component includes (i) polyethylene, or (ii) polypropylene, or (iii) polyethylene and polypropylene. In certain implementations a retention volume (e.g., absorbs a maximum fluid volume) of a transfer pad is of about 20 microliters to about 1 milliliter, or about 50 microliters to about 500 microliters, or about 100 microliters to about 300 microliters, or about 100, 110, 120, 130, 140, 150, 160, 170, 180, 190, 195, 200, 205, 210, 215, 220, 230, 240, 250, 260, 270, 280, 290 or 300 microliters. A lateral length of a transfer pad sometimes is the major dimension of the transfer pad (i.e., the lateral length is parallel to lateral axis 198 shown in FIG. 10), and the lateral length of the transfer pad often is greater than the transverse width of the transfer pad at the transfer pad distal terminus and proximal terminus. A transfer pad sometimes has a lateral length of about 8 millimeters (mm) to about 16 mm (e.g., about 10, 11, 12, 13, 14 or 15 mm); a transverse width of about 4 mm to about 10 mm (e.g., about 5, 6, 7, 8, 9 or 10 mm); and a longitudinal height of about 1 mm to about 5 mm (e.g., about 1, 2, 3, 4 or 5 mm). A transfer pad sometimes has an average (e.g., mean, median) pore size (e.g., pore diameter) of about 2 micrometers to about 100 micrometers, or about 10 micrometers to about 40 micrometers, or about 20 micrometers to about 30 micrometers, or about 23 micrometers to about 28 micrometers (e.g., about 2, 3, 4, 5, 10, 15, 16, 17, 18, 19, 20, 21, 22, 23, 24, 25, 26, 27, 28, 29, 30, 32, 33, 35, 40, 45, 50, 55, 60, 70, 80, 90, 100 micrometers), sometimes with a minimum pore size (e.g., pore diameter) of about 0.1 micrometers to about 10 micrometers, or about 0.5 micrometers to about 5 micrometers, or about 1 micrometer to about 3 micrometers (e.g., 0.1, 0.2, 0.3, 0.4, 0.5, 0.6, 0.7, 0.8, 0.9, 1.0, 1.2, 1.4, 1.6, 1.8, 2.0, 2.2, 2.3, 2.4, 2.6, 2.8, 3.0, 3.5, 4.0, 4.5, 5.0, 5.5, 6.0, 6.5, 7.0, 8.0, 9.0 or 10 micrometers), and sometimes with a maximum pore size (e.g., pore diameter) of about 5 micrometers to about 200 micrometers, or about 40 micrometers to about 80 micrometers, or about 50 micrometers to about 70 micrometers (e.g., about 5, 10, 20, 30, 40, 50, 55, 56, 57, 58, 59, 60, 61, 62, 63, 64, 65, 70, 80, 90, 95, 100, 120, 140, 160, 180, 200 or 300 micrometers).

When present in a test device component, the one more porous cartridge pads sometimes each independently include a circular proximal surface and a circular distal surface. The circular proximal surface and the circular distal surface sometimes independently have a diameter of about 1 millimeters (mm) to about 12 mm, or about 6 mm to about 10 mm, or about 1, 2, 3, 4, 5, 6, 7, 8, 9, 10, 11 or 12 mm. In certain instances, the one or more porous cartridge pads each independently include (i) polyethylene, or (ii) polypropylene, or (iii) polyethylene and polypropylene, or (iv) glass fiber, or (v) other organic or synthetic fibrous pad. In certain implementations, the one or more porous cartridge pads each independently have an average (e.g., mean or median) pore size (e.g., pore diameter) of about 0.05 micrometers to about 5 micrometers, or about 0.20 micrometers to about 2 micrometers, or about 0.45 micrometers to about 1 micrometer, or about 0.05, 0.06, 0.07, 0.08, 0.09, 0.1, 0.2, 0.3, 0.4, 0.5, 0.6, 0.7, 0.8, 0.9, 1, 2, 3, 4 or 5 micrometers.

In certain implementations, the transfer pad and the one or more optional cartridge pads independently include one or more reagents. The one or more reagents can be chosen from an analyte binding agent, buffering agent, surfactant, cell lysis agent, detergent, protein, enzyme, antibody or antigen-binding fragment thereof, antigen, binding pair member, bulking agent, water-absorbing agent, saccharide, polysaccharide, nucleic acid amplification reagent, nucleic acid and aptamer. An analyte binding agent can be any suitable agent for binding to an analyte for detection in a test strip (e.g., a nitrocellulose test strip). An analyte binding agent in certain implementations is an antibody or antigen-binding fragment thereof. An analyte binding agent sometimes binds to one of the following non-limiting examples of analytes: a hormone (e.g., testosterone, cortisol, melatonin); a vitamin (e.g., vitamin D); an infectious disease antigen; and a polymerase chain reaction nucleic acid amplicon (e.g., generated from and incorporating labeled primers). Any suitable binding pair member for use with a lateral flow strip can be utilized, non-limiting examples of which include antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA) and the like. Any suitable enzyme can be utilized, including but not limited to a ligase, polymerase, transposase, reverse transcriptase. A suitable water-absorbing agent can be utilized, such as a polysaccharide, for example. In certain implementations, the transfer pad includes a water-absorbing agent at the distal surface of the pad, and/or in a portion of the pad adjacent to the distal surface of the pad. One or more reagents can be distributed in all or a portion of the transfer pad and/or the one or more optional cartridge pads.

A reagent (e.g., a protein, enzyme, antibody or antigen) sometimes is modified with a detectable label. Any detectable label suitable for use with a lateral flow strip can be utilized, non-limiting examples of which include fluorescent labels such as organic fluorophores, lanthanide fluorophores (chelated lanthanides; dipicolinate-based Terbium (III) chelators), transition metal-ligand complex fluorophores (e.g., complexes of Ruthenium, Rhenium or Osmium); quantum dot fluorophores, isothiocyanate fluorophore derivatives (e.g., FITC, TRITC), succinimidyl ester fluorophores (e.g., NHS-fluorescein), maleimide-activated fluorophores (e.g., fluorescein-5-maleimide), amidite fluorophores (e.g., 6-FAM phosphoramidite); protein fluorphores (e.g., phycobiliprotein (e.g., R-phycoerythrin), green fluorescent protein (GFP), red fluorescent protein (RFP)); radioactive isotopes (e.g., I-125, I-131, S-35, P-31, P-32, C-14, H-3, Be-7, Mg-28, Co-57, Zn-65, Cu-67, Ge-68, Sr-82, Rb-83, Tc-95m, Tc-96, Pd-103, Cd-109, and Xe-127); particles, such as nanoparticles and/or colloidal particles, for example (e.g., gold, latex, silver or carbon particles (e.g., nanoparticles)); light scattering or light diffracting labels (e.g., light scattering gold nanorods, resonance light scattering particles); an enzymic or protein label (e.g., GFP, RFP, peroxidase); or other chromogenic label or dye (e.g., cyanine). Non-limiting examples of organic fluorophores include xanthene derivatives (e.g., fluorescein, rhodamine, Oregon green, eosin, Texas red); cyanine derivatives (e.g., cyanine, indocarbocyanine, oxacarbocyanine, thiacarbocyanine, merocyanine); naphthalene derivatives (dansyl, prodan derivatives); coumarin derivatives; oxadiazole derivatives (e.g., pyridyloxazole, nitrobenzoxadiazole, benzoxadiazole); pyrene derivatives (e.g., cascade blue); oxazine derivatives (e.g., Nile red, Nile blue, cresyl violet, oxazine 170); acridine derivatives (e.g., proflavin, acridine orange, acridine yellow); arylmethine derivatives (e.g., auramine, crystal violet, malachite green); and tetrapyrrole derivatives (e.g., porphin, phtalocyanine, bilirubin). A detectable label sometimes includes a fluorophore or a dye.

A lateral flow strip in a test device component can be any type of strip, and of any dimension, suitable for analysis of analytes in sample fluid transmitted from a sample collection device component to the transfer pad and from the transfer pad to the strip. A lateral flow strip also is referred to herein as a test strip. The strip includes nitrocellulose in certain instances, can include one or more associated pads (e.g., polyesters pad, glass fiber pad, absorbent pad) and often includes one or more reagents. Any suitable reagent(s) can be incorporated onto or into a strip and a reagent sometimes is bound (e.g., covalently bound) to the strip. Non-limiting examples of reagents that can be incorporated into or onto a strip include a blocking agent, nucleic acid (e.g., capture oligonucleotide probes, DNA fragment, RNA fragment), a binding pair member, a protein, enzyme, antibody or antigen-binding fragment thereof or antigen or other reagent described herein. In certain implementations, antibodies or fragments thereof can be utilized for a sandwich assay and protein-hapten conjugates can be utilized for a competitive assay. A test device component sometimes includes a strip pad in contact with the distal terminus of a test strip and/or a region on the anterior and/or posterior surface of the test strip adjacent to the distal terminus of the strip. Such a strip pad generally is porous and absorbent, and can facilitate fluid flow, in the proximal to distal direction, through the strip. In certain implementations, a test strip is substituted with another type of detection element, such as an absorbent pad or a matrix disposed on a support (e.g., silica gel, alumina or other matrix coated on a glass, metal, plastic or other support), for example.

In certain implementations, (i) a portion of the transfer pad that is disposed adjacent to a portion of the strip, and (ii) the portion of the test strip disposed adjacent to the portion of the transfer pad, are separated. In certain implementations, the portions (i) and (ii) are separated by a removable barrier, where the removable barrier is disposed between the portion of the transfer pad (i) and the portion of the test strip adjacent to the transfer pad (ii). In certain instances, a portion of the transfer pad posterior surface is disposed opposite a portion of the test strip anterior surface, and the removable barrier is disposed between the portion of the transfer pad posterior surface and the portion of the test strip anterior surface opposite to the portion of the transfer pad posterior surface. The removable barrier can be a pull tab. A pull tab sometimes is manufactured from a flexible and thin and non-porous material, such as a plastic, for example. A pull tab can include any suitable plastic, including without limitation polyethylene terephthalate (PET), low density polyethylene (LDPE), medium density polyethylene (MDPE), polypropylene (PP), vinyl, polyvinyl chloride (PVC), or combination thereof (e.g., a pull tab can be punched from a plastic sheet). In certain implementations, a portion of the test device housing includes a slot through which a portion of the removable barrier protrudes and extends from an exterior surface of the test device housing.

In certain implementations, the portions (i) and (ii) described in the foregoing paragraph are separated by a gap (i.e., a void). In certain instances, a button is disposed in the test device housing opposite the portion of the transfer pad disposed adjacent to the portion of the test strip. In certain implementations, a portion of the transfer pad posterior surface is disposed opposite a portion of the test strip anterior surface, and the button is disposed opposite the portion of the transfer pad. A button sometimes is defined by slits in the test device housing. In certain instances, the button is defined by three slits in the test device housing and a portion of the test device housing sidewall opposite one of the slits functions as a living hinge. A living hinge portion can include a kerf that facilitates operation of the living hinge, and a device housing sometimes includes no kerf at the living hinge. A button can be disposed on an anterior portion of the test device housing. In certain implementations, the test device housing includes an extension bar extending from an interior surface of the button and the test device housing, which includes a poster surface. The posterior surface of the extension bar often is disposed opposite the portion of the transfer pad disposed adjacent to the test strip. The posterior surface of the extension bar sometimes contacts a portion of the anterior surface of the transfer pad and sometimes the posterior surface of the extension bar is spaced from the anterior surface of the transfer pad. In certain instances, the test device housing includes a catch, and the extension bar includes a catch counterpart (e.g., a depression or projection disposed in or on a side of the extension bar) disposed adjacent to the catch in the test device housing.

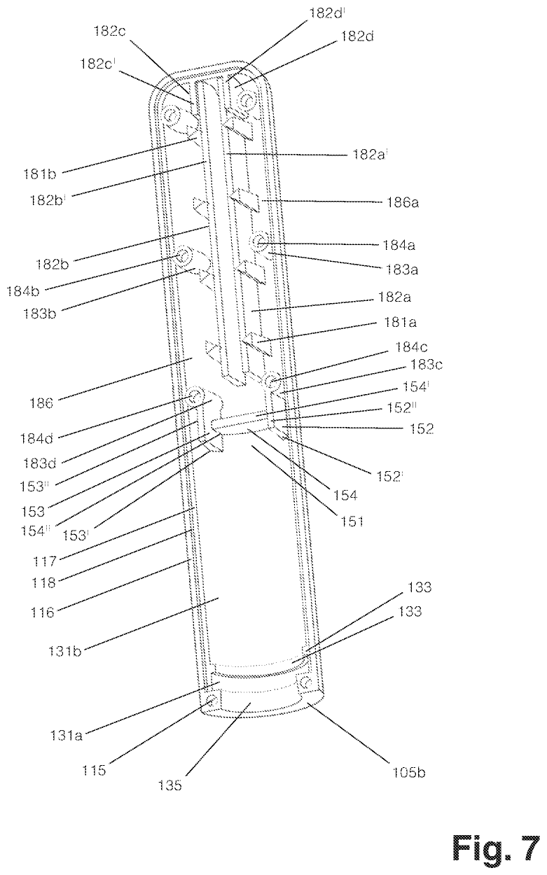

A non-limiting example of a test device component is test device component 100 illustrated in FIG. 1 and FIG. 4A to FIG. 12B. Test device component 100 includes test device housing 101 illustrated in FIG. 1, for example. Test device housing 101 includes a cylindrical first region 195, a lateral planar cylindrical segment second region 197 and a lateral arcoid cylindrical segment third region 196 disposed between the first region and the second region, as illustrated in FIG. 10 and FIG. 11. As illustrated in FIG. 4A to FIG. 7, test device housing 101 includes posterior member 102 (also referred to as test device housing (bottom)), anterior member 103 (also referred to as test device housing (top)), and junction 104 between the posterior member and the interior member. Test device housing 101, posterior member 102 and anterior member 103 each include a proximal terminus (105, 105a, 105b), a distal terminus (106, 106a, 106b) and wall exterior surface (108a, 108b, 108c, 108d for anterior member 103 and 109a, 109b, 109c for posterior member 102).

A number of features align and connect posterior member 102 and anterior member 103 in test device housing component 101. Edge members of anterior member 103 fit into and contact edge members of posterior member 102. Edge members of anterior member 103 include recessed protrusion 113, having edge 113a and a surface 113b adjacent to recessed protrusion 113. Edge members of posterior member 102 include edge 116, recessed edge 117 and sidewall 118 between edge 116 and recessed edge 117. Surface 113b of anterior member 102 contacts sidewall 118 of posterior member 102, which in part constrict lateral and transverse movement of anterior member 103 relative to posterior member 102. Connectors in anterior member 103 (projections 114a, 114b, 114c) connect to connector counterparts in posterior member 102 (receptacles 115, 184a, 184b, 184c, 184d) and in part restrict longitudinal movement of anterior member 103 relative to posterior member 102. A subset of connectors of anterior member 103 is disposed on edge 113a (connector 114a), another subset of connectors of anterior member 103 is disposed on interior sidewall 121c (connector 114b), and another subset of connectors of anterior member 103 is disposed connector posts 119a, 119b (connector 114c). A subset of connectors of posterior member 102 is disposed on edge 116 (receptacle 115) and another subset of connectors of posterior member 103 is disposed on connector posts 183a, 183b, 183c, 183d (receptacles 183a, 183b, 183c, 183d).

Posterior member 102 of test device housing 101 includes base projection 107, having proximally disposed portion 107b and distally disposed portion 107a. Base projection 107 can serve as a platform or foot. An operator can place the posterior surface of base projection 107 of test device component 100 (or assembly 300) on a surface. Where the surface is a flat, horizontal surface, such placement can orient test device 100 (or assembly 300) in a laterally-disposed orientation, and can reduce the likelihood that test device component 100 (or assembly 300) rolls on or off the surface.

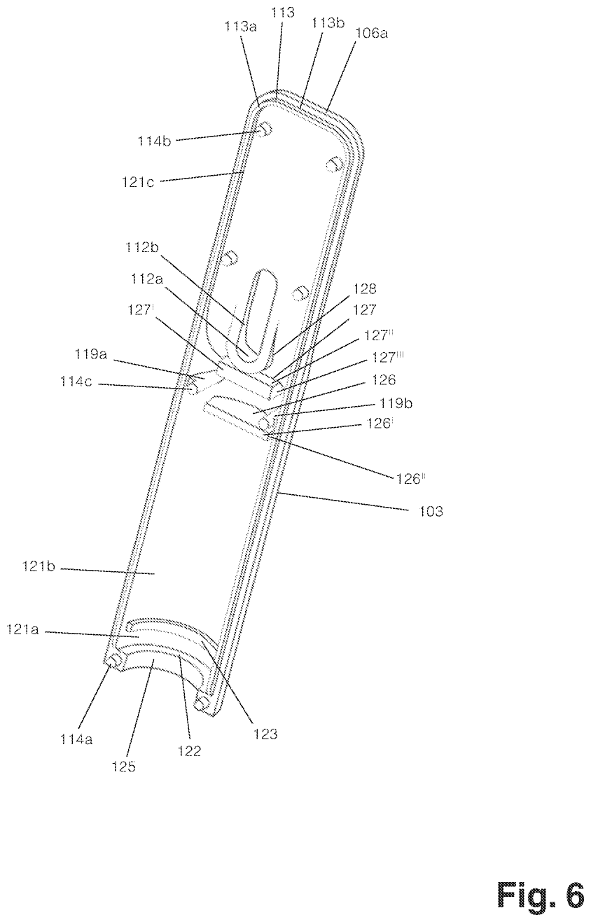

Posterior member 102 of test device housing 101 includes window 110 (also referred to as results viewing window) defined by recess 112. Recess 112 is in part defined by interior wall surfaces 112a and 112b within interior window projection 128 extending from interior surface 112b, and by the wall thickness disposed at window 110 (108b and 108d), as illustrated in FIG. 6.

Test device housing 100 includes proximal opening 120 (also referred to as a sample collector port), at which test device housing proximal opening rim 125 is disposed in anterior member 103 and test device housing proximal opening rim 135 is disposed in posterior member 102. Rim member 125 steps to interior surface 121 (121a, 121b, 121c) via annular recessed surface 122 in anterior member 103. Annular protrusion 123 projects from interior surface 121 adjacent to rim 125, with interior wall surface 121a disposed between annular protrusion 123 and annular recessed surface 122. Rim member 135 steps to interior surface 131 (131a, 131b) via annular recessed surface 132 in posterior member 102. Annular protrusion 133 projects from interior surface 131 adjacent to rim 135, with interior wall surface 131a disposed between annular protrusion 133 and annular recessed surface 132.

Projecting from interior surface 121 of anterior member 103 is test device housing second extension wall 126 having posterior edge 126′ and side edge 126″ (callout 126 is to the distally-disposed face of the second wall). The major dimension of posterior edge 126′ is transversely oriented, and the transverse length of the proximally-disposed face and the distally-disposed face of second wall 126 at edge 126′ is the major dimension of second wall 126. The transverse length of edge 126′ of second wall 126 is parallel to transverse axis 194 illustrated in FIG. 4D.

Projecting also from interior surface 121 of anterior member 103 is extension bar 127, having posterior edge 127′, distally-disposed face 127″ and side edge 127′″. The major dimension of posterior edge 127′ is transversely oriented, and the transverse length of the proximally-disposed face and the distally-disposed face of extension bar 127 at edge 127′ is the major dimension of extension bar 127. The transverse length of edge 127′ of extension bar 127 is parallel to transverse axis 194 illustrated in FIG. 4D.

Projecting from interior surface 151 of posterior member 102 is third extension wall 152, having proximal side 152′ and anterior edge 152″. Also projecting from interior surface 151 of posterior member 102 is fourth extension wall 153, having proximal side 153′ and anterior edge 153″. Callout 153 designates a face of the fourth extension wall opposing the transverse center (designated by callout 151) of posterior member 102. Dimensions of third extension wall 152 and fourth extension wall 153 are the same or about the same. The major dimension of anterior edge 152″ of third extension wall 152 is laterally disposed, and the lateral length of the face of third extension wall 152 opposing the transverse center of posterior member 102 and disposed at anterior edge 152″, is the major dimension of third extension wall 152. The major dimension of anterior edge 153″ of fourth extension wall 153 is laterally disposed, and the lateral length of the face of fourth extension wall 153 opposing the transverse center of posterior member 102 and disposed at anterior edge 153″, is the major dimension of fourth extension wall 153. The lateral length of anterior edge 152″ of third extension wall 152, and the lateral length of anterior edge 153″ of fourth extension wall 153, is parallel to lateral axis 198 illustrated in FIG. 10. Proximal side 152′ of third extension wall 152, and proximal side 153′ of fourth extension wall 153, each is longitudinally-oriented (i.e., parallel to longitudinal axis 199 shown in FIG. 10). The longitudinally-disposed and distally-disposed terminal side of third extension wall 152 is integrated with connector member post 183c. The longitudinally-disposed and distally-disposed terminal side of fourth extension wall 153 is integrated with connector member post 183d. The longitudinally-disposed height of third extension wall 152 is the same as or about the same as the longitudinally-disposed height of fourth extension wall 153.

Projecting also from interior surface 151 of posterior member 102 is first extension wall 154, having anterior edge 154′ and side 154″. The major dimension of anterior edge 154′ is transversely oriented, and the transverse length of the proximally-disposed face and the distally-disposed face of first extension wall 154 at edge 154′ is the major dimension of first extension wall 154. The transverse length of edge 154′ of first extension wall 154 is parallel to transverse axis 194 illustrated in FIG. 4D. Side 154″ of first extension wall 154 is longitudinally-oriented (i.e., parallel to longitudinal axis 199 shown in FIG. 10). Side 154′ is integrated with a portion of the face of fourth extension wall 153 opposing the transverse center of posterior member 102, and the longitudinally-disposed side of first extension wall 154 opposing side 154′ is integrated with a portion of the face of third extension wall 152 opposing the transverse center of posterior member 102 (designated by callout 151). The longitudinally-disposed height of first extension wall 154 is less than the longitudinally-disposed height of third extension wall 152 and fourth extension wall 153 (the anterior edge of third extension wall 152 and the anterior edge of fourth extension wall 153 are proud of the anterior edge of first extension wall 154.

Projecting from interior surface 186 (and 186a) of posterior member 102 are containment posts 181a and 181b configured to retain a lateral flow strip and constrain transverse movement of the strip. The longitudinally-disposed length of each of containment posts 181a and 182b is the major post dimension and the anterior terminal face of each post has a triangular profile. A point of the triangular profile faces the transverse center of posterior member 102 (designated by callout 151), and the triangular point facing the transverse center of each of posts 181a and 181b is spaced from the transverse center by a distance equal to half or about half of the transverse width of the lateral flow strip contained by the containment posts. The triangular point of each of posts 181a and 181b facing the transverse center extends as a longitudinally-disposed edge also facing the transverse center of posterior member 102.

Projecting also from interior surface 186 (and 186a) of posterior member 102 are support ribs 182a, 182b, 182c and 182d, having support rib edges 182a′, 182b′, 182c′ and 182d′, respectively.

Support ribs 182a and 182b are configured to contact posterior surface 185d of lateral flow strip 185. The major dimension of anterior edge 182a′ of support rib 182a is laterally disposed, and the major dimension of anterior edge 182b′ of support rib 182b is laterally disposed. Anterior edge 182a′ and anterior edge 182b′ typically are disposed at the same longitudinal height relative to the interior surface 186a from which ribs 182a and 182b project. One face of support rib 182a opposes the transverse center of posterior member 102 (designated by callout 151), and is referred to as an internal face. The other face of rib 182a opposing the internal face is referred to as an external face. The face of support rib 182b opposing the internal face of support rib 182a also opposes the transverse center of posterior member 102, and is referred to as an interior face. The other face of rib 182b opposing the internal face is referred to as an external face. The internal face of each of ribs 182a and 182b is spaced from the transverse center by a distance that is the same or about the same from the proximal terminus to the distal terminus of each of ribs 182a and 182b. The longitudinally-disposed edge facing the transverse center of posterior member 102 of containment posts 181a can contact the external face of support rib 182a. Similarly, the longitudinally-disposed edge facing the transverse center of posterior member 102 of containment posts 181b can contact the external face of support rib 182b. Anterior edge 182a′ and anterior edge 182b′ typically are disposed at the same longitudinal height relative to the interior surface 186a from which ribs 182a and 182b project.

Support ribs 182c and 182d are integrated with and extend from (i) interior surface 186a and (ii) the interior surface of the distal terminus of posterior member 102. Support rib 182c is adjacent to support rib 182b with the internal face of rib 182c opposing the external face of rib 182b. The internal face of rib 182c sometimes contacts the external face of rib 182b and sometimes is spaced from the external face of rib 182b. Support rib 182d is adjacent to support rib 182a with the internal face of rib 182d opposing the external face of rib 182a. The internal face of rib 182d sometimes contacts the external face of rib 182a and sometimes is spaced from the external face of rib 182a. The major dimension of each of anterior edge 182c′ and anterior edge 182d′ is laterally disposed. Anterior edge 182c′ and anterior edge 182d′ typically are disposed at the same longitudinal height relative to the interior surface 186a from which ribs 182c and 182d project. Anterior edge 182c′ and 182d′ typically are raised (i.e., are proud) with respect to anterior edge 182a′ and anterior edge 182b′. Support ribs 182c and 182d are configured to guide the test strip during assembly and retain the test strip after assembly in its intended position.

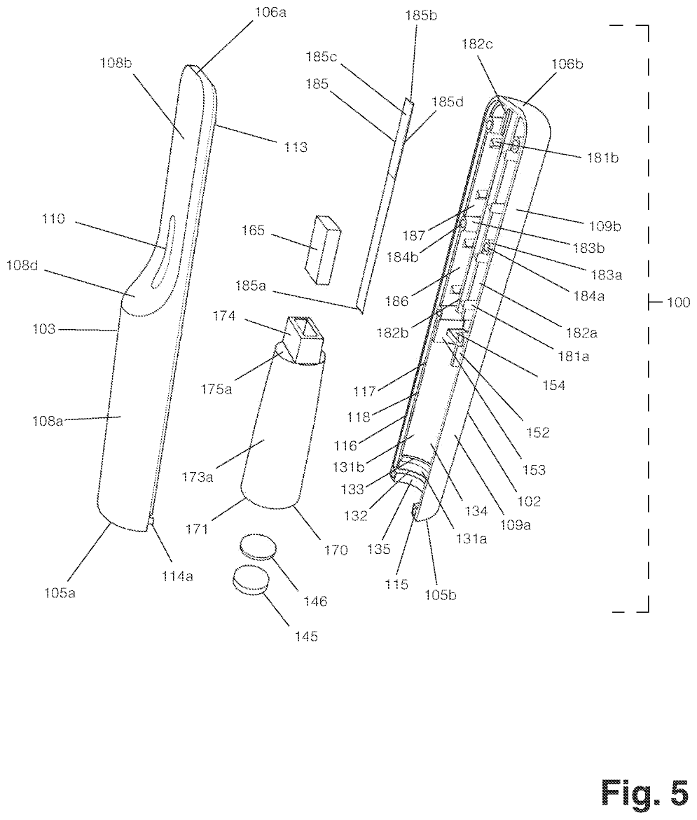

Within test device housing 101 is cartridge housing 170 (also referred to as a sample process cartridge housing). The entirety of cartridge housing 170 resides in test device housing 101 of test device component 100. In certain implementations, a portion of a cartridge can be disposed outside of test device housing 101 (e.g., shank 479a of test device component 400 illustrated in FIG. 18). As illustrated in FIG. 5 and FIG. 8 to FIG. 12B, cartridge housing 170 includes proximal terminus 171 with proximal edge 171a, distal terminus 172 and sidewall 173, with sidewall exterior surface 173a and interior surface 173b. Cartridge housing 170 is a tube defined by circular cross sections, is hollow and includes an interior lumen (173d, 173e). The cross-sectional diameter of cartridge housing 170 reduces from proximal terminus 171 to distal terminus 172. Cartridge housing 170 includes two frustum portions. A proximally-disposed first frustum portion is defined by sidewalls having a first exterior draft angle, and another distally-disposed second frustum portion contiguous with the first frustum portion is defined by sidewalls having a second exterior draft angle, where the second draft angle is greater than the first draft angle. The first frustum portion transitions to the second frustum portion at sidewall transition 173c, and a cross-sectional diameter in the first frustum portion is greater than a cross-sectional diameter in the second frustum portion.

At distal terminus 172 of cartridge housing 170 is terminal wall 175 (also referred to as a restriction structure), having exterior surface 175a and interior surface 175b. Interior surface 175a of terminal wall 175 is coextensive and integrated with sidewall interior surface 173b at distal terminus 172 of the cartridge housing. The diameter of terminal wall 175 (exterior diameter and interior diameter) is transversely disposed (i.e., parallel to transverse axis 194 shown in FIG. 4D). While the interior diameter of terminal wall interior surface 175b defines a terminal wall surface area between 60 square millimeters (mm) and 61 square mm in cartridge housing 170, the interior wall surface area of a terminal wall can have any suitable surface area.

Terminal wall 175 includes bores 177a and 177b that traverse the entire thickness of the terminal wall and include an aperture on each of interior surface 175b and interior surface 175a. At least a portion of the surface area, or the entire surface area, of each of bores 177a and 177b is aligned with interior 179 of terminal projection 174. While bores 177a and 177b each are defined at terminal wall 175 by a predominantly rectilinear perimeter with one curved side, the bores can be defined by any suitable perimeter that permits transmission of sample fluid in the proximal to distal direction from the cartridge housing. While each of bores 177a and 177b at terminal wall 175 has a bore surface area between 6 square millimeters (mm) and 7 square mm, for a combined bore surface area between 12 square mm and 14 square millimeters, for cartridge housing 170, any suitable bore surface area can be implemented for transmission of sample fluid in the proximal to distal direction from the cartridge housing. For cartridge housing 170, the ratio of (i) the combined bore 177a and 177b surface area to (ii) the surface area of the terminal wall interior surface 175b, is between about 0.20 to about 0.23.

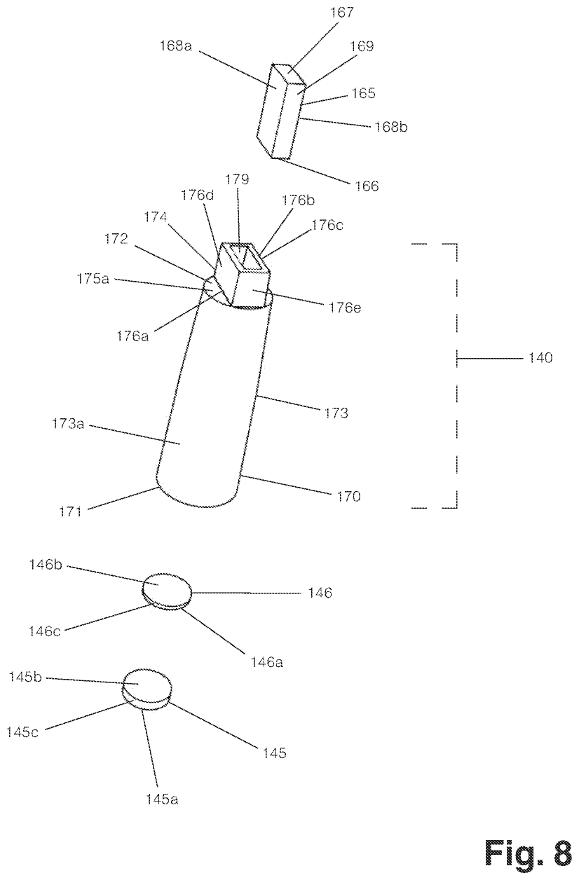

Extending from distal terminus 172 and from terminal wall 175 exterior surface 175a of cartridge housing 170 is cartridge terminal projection 174, having proximal terminus 176a and distal terminus 176b. Terminal projection 174 is a hollow tube and is configured to retain a portion of transfer pad 165. Transfer pad 165 (also referred to as a transport pad) has a rectangular cuboid geometry, having proximal terminus (proximal surface) 166, distal terminus (distal surface) 167, anterior surface 168a and posterior surface 168b, and side surfaces 169 bounded by the foregoing transfer pad surfaces, as illustrated in FIG. 8. Terminal projection 174 has a rectangular cross section for retaining transfer pad 165, and includes posterior side member exterior surface 176c, anterior side member exterior surface 176d, side member exterior surface 176e and interior 179, as shown in FIG. 8. While the portion of transfer pad 165 disposed within interior 179 of terminal projection 174 is retained by an interference fit, the transfer pad could be retained in any other suitable manner (e.g., weld and the like).

Cartridge housing 170 includes a first cartridge housing pad 145 (also referred to as filtration/chemistry pad) that is proximally-disposed with respect to, and contacts, an optional second cartridge housing pad 146 (also referred to as conjugate/chemistry pad). Each cartridge housing pad is dimensioned to fit within the cartridge housing interior with a side of each pad contacting a portion of sidewall interior surface 173b. Each cartridge housing pad is porous and at least a continuous annular portion of the side of each pad continuously typically contacts an annular portion of interior sidewall surface 173b. A cylindrical cartridge housing pad sometimes is deformed to facilitate such a continuous fit in a frustum-shaped interior of cartridge housing 170. First cartridge housing pad 145 includes proximal surface 145a, distal surface 145b and side surface 145c. Second cartridge housing pad 146, includes proximal surface 146a, distal surface 146b and side surface 146c. Within cartridge housing 170, distal surface 145b of first cartridge housing pad contacts proximal surface 146a of second cartridge housing pad 146, and distal surface 146b of second cartridge housing pad 146 contacts interior surface 175b of terminal wall 175.

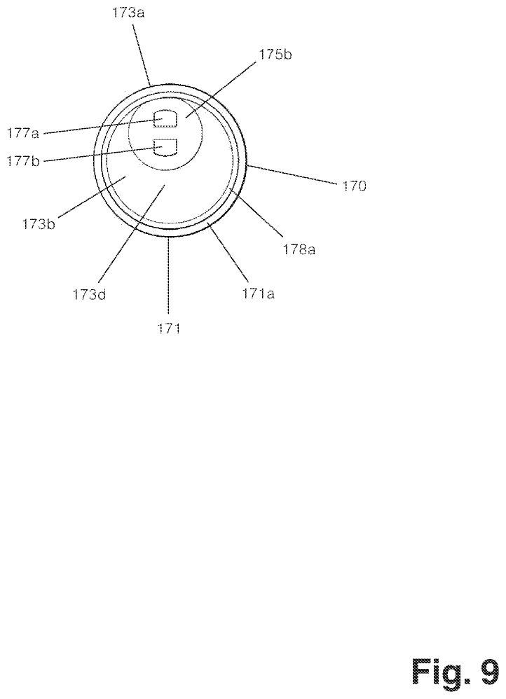

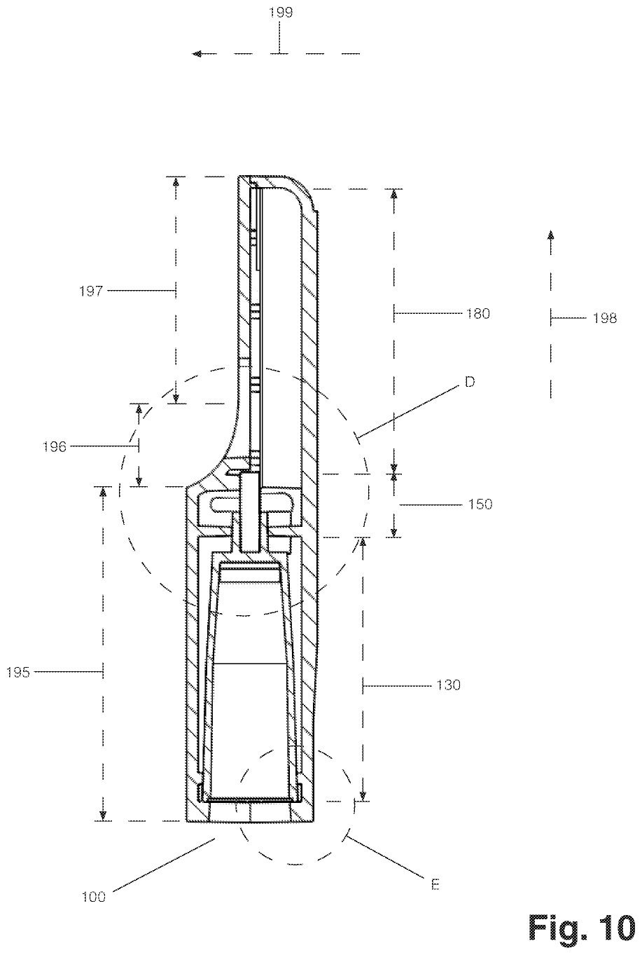

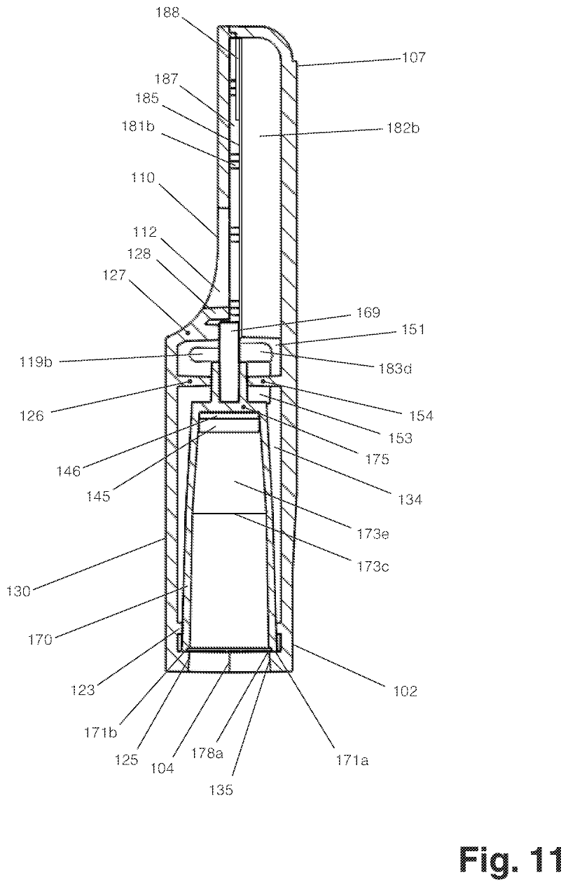

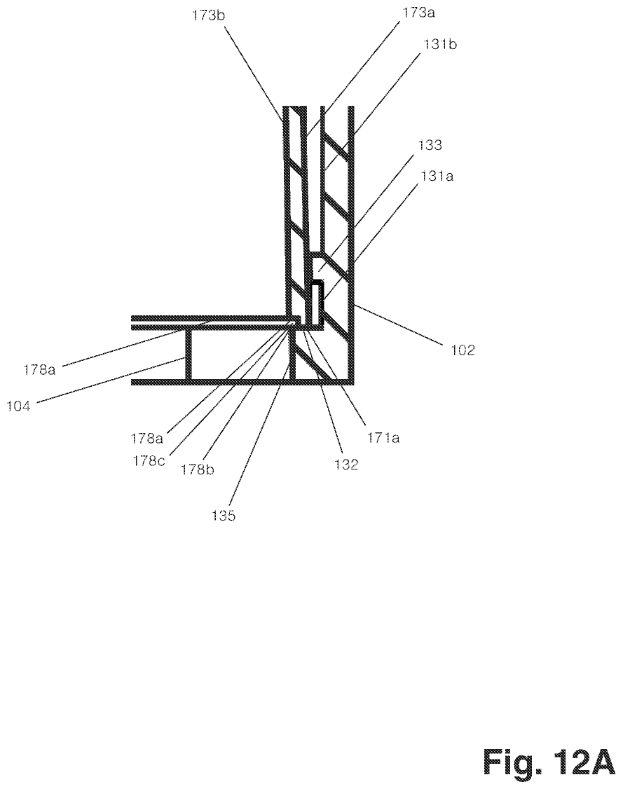

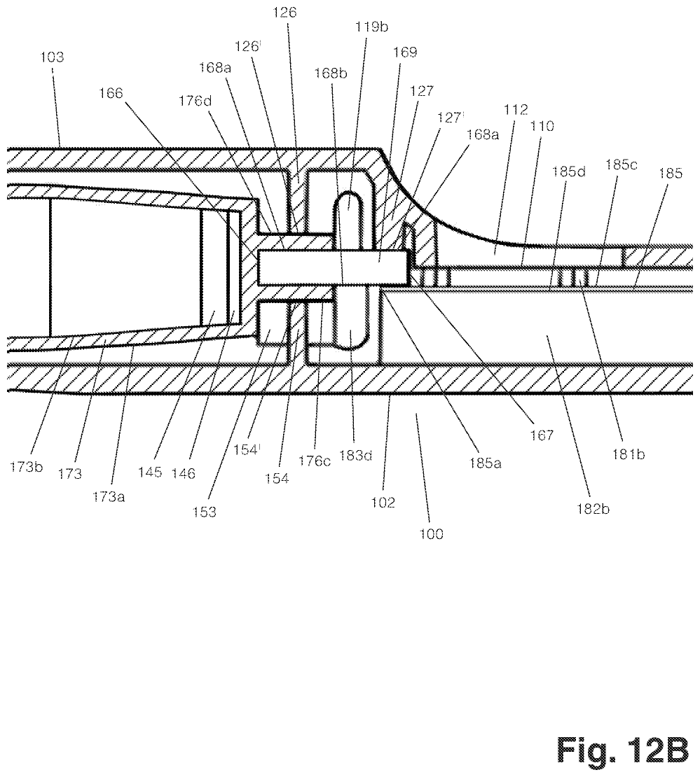

For test device component 100, orientation of test device housing 101, cartridge housing 170, cartridge housing pads 145 and 146, terminal projection 174 and transfer pad 165, and lateral flow test strip 185 is illustrated in FIG. 10 to FIG. 12B. Cartridge housing 170 is retained within the interior of test device housing 101. As shown in FIG. 12A, cartridge housing proximal edge 171a contacts test device housing annular recessed surface 132 (and 122) of rim 135 (and 125), thereby constraining lateral movement of the cartridge. Annular groove 178c is formed between recessed surface 132 (and 122) of test device housing 101 and recessed surface 178a of cartridge housing 170, in which rim surface 178b forms the floor of annular groove 178c. Annular groove 178c functions as a connector member counterpart that receives stem annular protrusion 218 connector member of sample collection device 200.

As shown in FIG. 12B, anterior edge 154′ of first extension wall 154 of test device housing 101 contacts posterior surface 176c of terminal projection 174 of cartridge housing 170, and posterior edge 126′ of second extension wall 126 of test device housing 101 contacts anterior surface 176d of terminal projection 174 of cartridge housing 170, thereby constraining longitudinal movement of cartridge housing 170 within test device housing 101. A portion of the internal face of third extension wall 152 of test device housing 101 contacts a posteriorly-disposed portion of side surface 176e of terminal projection 174 of cartridge housing 170, and a portion of the internal face of fourth extension wall 153 of test device housing 101 contacts a posteriorly-disposed portion of the exterior side surface opposite side surface 176e of terminal projection 174 of cartridge housing 170, thereby constraining transverse movement of cartridge housing 170 within test device housing 101. An a portion of proximal edge 152′ of third extension wall 152 of test device housing 101 contacts a portion of exterior surface 175a of terminal wall 175 of cartridge housing 170, and a portion of proximal edge 153′ of fourth extension wall 153 of test device housing 101 contacts another portion of exterior surface 175a of terminal wall 175 of cartridge housing 170, thereby constraining lateral movement of cartridge housing 170 within test device housing 101.

As shown in FIG. 10 and FIG. 11, test strip 185 (also referred to as a lateral flow strip) is disposed adjacent to transfer pad 165, and includes proximal terminus 185a, distal terminus 185b, anterior surface 185c and posterior surface 185d. A proximally-disposed portion of anterior surface 185c of test strip 185 contacts a distally-disposed portion of posterior surface 168b of transfer pad 165 at a transfer pad-test strip interface. Test strip absorbent pad 188 contacts a distally-disposed portion of anterior surface 185c of test strip 185, and without being limited by theory, facilitates fluid transmission through test strip 185.

As shown in FIG. 12B, posterior surface 185d of lateral flow test strip 185 contacts and is supported by edge 182a′ of support rib 182a, and edge 182b′ of support rib 182b, of test device housing 101. Portions of each side edge of test strip 185 extending from proximal terminus 185a to distal terminus 185b contact (i) internal edges of containment posts 181a and 181b of test device housing 101, and (ii) an anteriorly-disposed portion of the internal face of each of support ribs 182c and 182d, thereby constraining transverse movement of test strip 185 in test device housing 101, and optionally constraining longitudinal movement of test strip 185 in test device housing 101 depending on tightness of fit between surfaces.

As shown in FIG. 12B, posterior edge 127′ of extension bar 127 of test device housing 101 contacts a distally-disposed portion of anterior surface 168a of transfer pad 165. Posterior edge 127′ of extension bar 127 optionally exerts pressure in the anterior to posterior direction to the portion of the transfer pad contacting posterior edge 127′, and from the portion of transfer pad posterior surface 168b to the portion of test strip anterior surface 185c opposed to posterior edge 127′, thereby constraining longitudinal movement of test strip 185, transfer pad 165 and cartridge housing 170 in test device housing 101. Optional pressure exerted by extension bar 127 can assure contact of transfer pad 165 uniformly along the transfer pad-test strip interface. Without being limited by theory, optional pressure exerted by posterior edge 127′ of extension bar 127 can reduce and partially restrict fluid flow from transfer pad 165 to test strip 185 compared to implementations in which extension bar 127 does not apply pressure to transfer pad 165. Reduction of fluid flow by transfer pad 165 can reduce potential for flooding of test strip 185 and can facilitate transmission of fluid into test strip 185 for transmission of fluid within test strip 185 in the proximal to distal direction.

Also as shown in FIG. 12B, sidewall interior surface 121b, 131b of test device housing is spaced from sidewall exterior surface 173a of cartridge housing 170 within lumen 134 of test device housing 101. Anterior surface 185c of test strip 185 also is spaced from sidewall interior surface 121c within lumen 187 of test device housing 101. Also, a portion of anterior surface 185c of test strip 185 opposes window 110 disposed in test device housing 101, and is exposed to the exterior of test device housing 101.

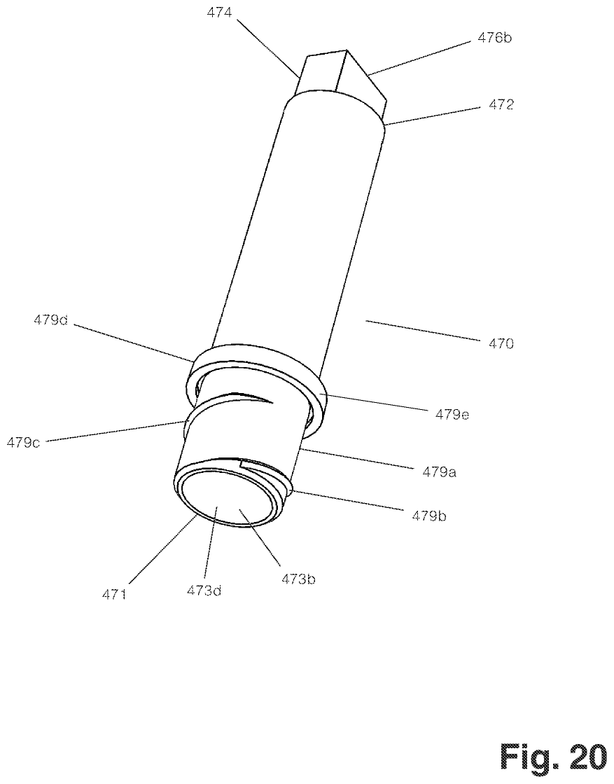

Non-limiting variations of test device 100 are illustrated for test devices 400, 700 and 1000. Test device 400 is similar to test device 100 except for the connector member that connects test device 400 to sample collection device 500. Test device 100 includes annular groove 178c as a connection member counterpart to annular groove 218 disposed on sample collection device 200. Test device 400 includes test device housing 101 and a test device cartridge housing 471, which substitutes annular groove 178c with a screw connector member for connection to sample collection device 500, as depicted in FIG. 18 and FIG. 20. Most features of cartridge housing 470 are shared with cartridge housing 170. Tubular cartridge housing 470 includes proximal terminus 471, distal terminus 472, sidewall interior surface 473b, and lumen 473d. Extending from distal terminus 472 of cartridge housing 470 is terminal projection 474, which includes distal terminus 476b and is dimensioned to retain transfer pad 165. Shank 479a extends from proximal terminus 471 of cartridge housing 470. A portion of shank 479a of test device cartridge housing 470 protrudes from proximal terminus 405 of test device housing 401 of test device 400. Shank 479a includes thread counterpart 479b (and portion 479c), which engages thread 509 of sample collection device 500, and can connect and optionally lock sample collection device 500 to test device 400 in assembly 600. The effective outer diameter of shank 479a and thread 479b is less than the interior diameter disposed at rim 425 and 435 of test device housing 401 (elements 401, 425 and 435 correspond to elements 101, 125 and 135). Cartridge housing 470 also includes collar 479d having collar exterior surface 479e that contacts interior surface 421a and 431a of test device housing 401, thereby restraining lateral movement of cartridge housing 470 in test device housing 401 (elements 401, 421a and 431a correspond to elements 101, 121a and 131a).

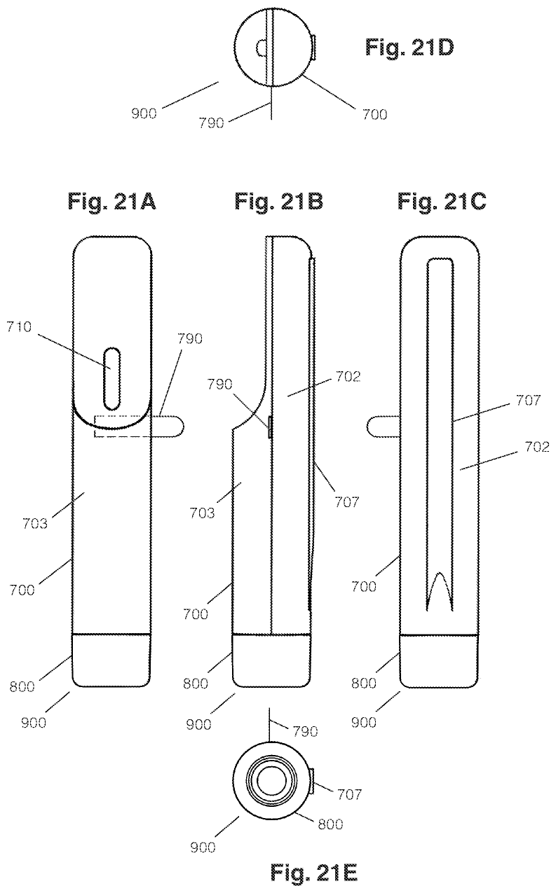

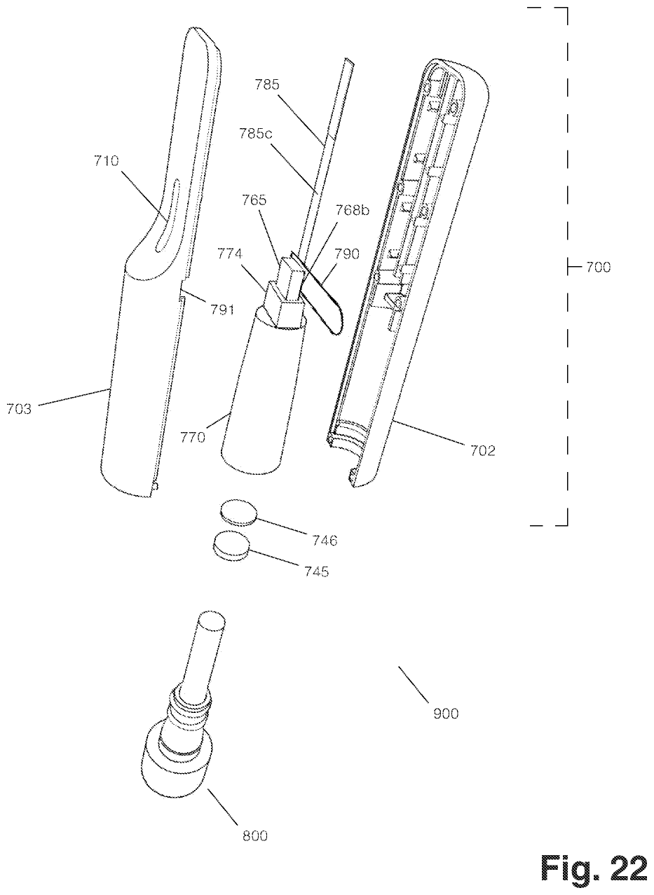

Test device 700 has the same features of test device 100 except that it includes a removable barrier between transfer pad 165 and test strip 185 at the transfer pad-test strip interface. The removable barrier of test device 700 is a pull tab that blocks fluid transmission from the transfer pad to the test strip until the portion of the pull tab at the transfer pad-test strip interface is removed. The removable barrier provides for operator control over the amount of time that sample fluid is retained in the transfer pad before sample fluid in the transfer pad is permitted to transmit from the transfer pad to the test strip, which can allow sufficient time for reagents in the transfer pad to interact with sample fluid analyte(s). As shown in FIG. 21A to FIG. 22, test device 700 includes test device housing posterior member 702, test device housing anterior member 703, test device housing base extension 707 and test device housing window 710 in the same orientation as counterparts in test device 100. Test device 700 also includes test device cartridge housing 770, having first cartridge housing pad 745, second cartridge housing pad 746, cartridge terminal projection 774, transfer pad 765 and test strip 785, in the same orientations as for counterparts in test device 100. Test device housing anterior member 703 includes slot 791 that extends through the wall of anterior member 703 at its perimeter. A portion of removable barrier 790 (pull tab) is disposed at the transfer pad-test strip interface between transfer pad posterior surface 768b and test strip anterior surface 785c, and another portion of removable barrier 790 is disposed in slot 791 and exists outside test device anterior member 703. The portion of removable barrier 790 existing outside test device anterior member 703 often is of a length sufficient for an operator to grasp and remove the barrier from the transfer pad-test strip interface.

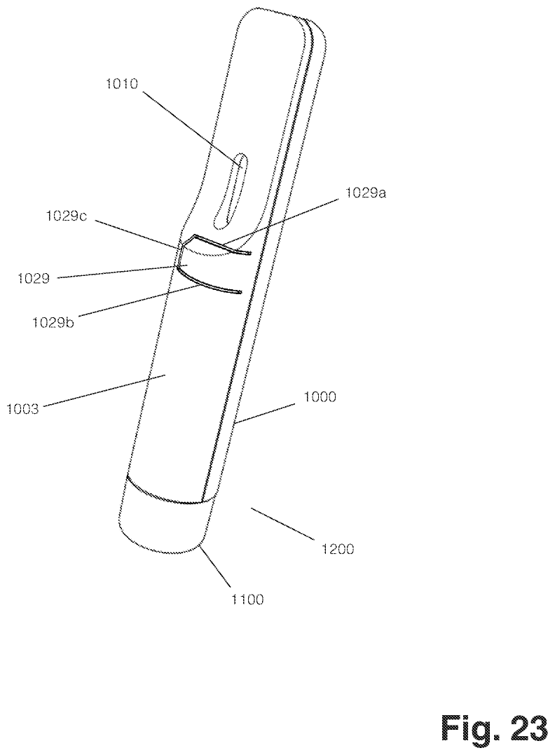

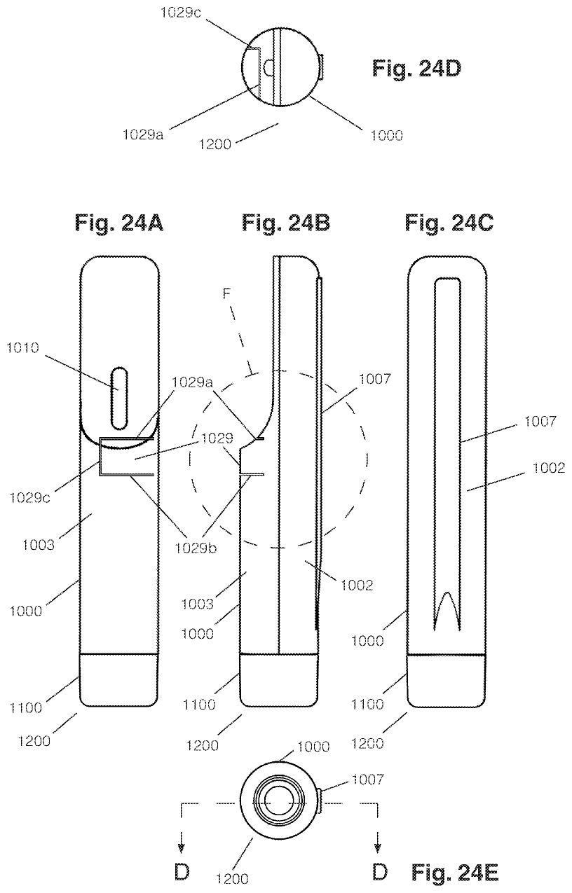

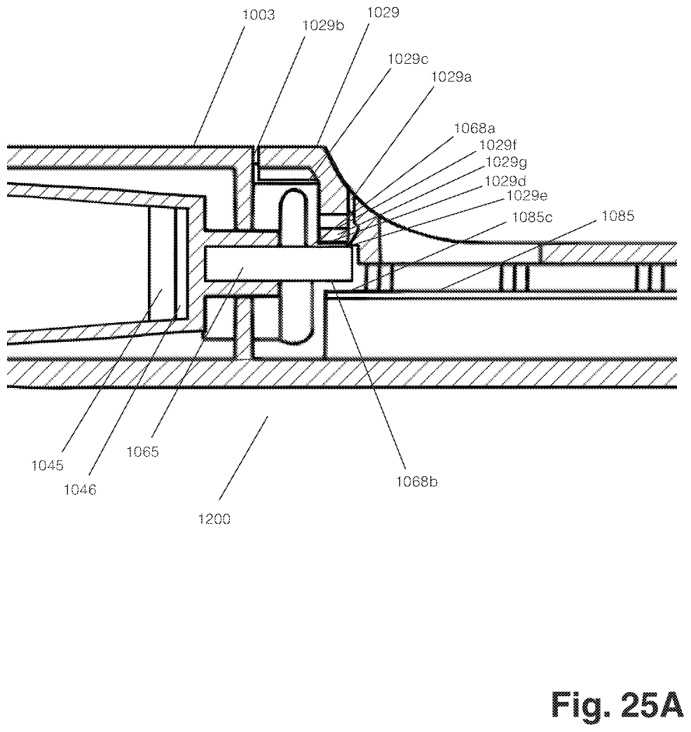

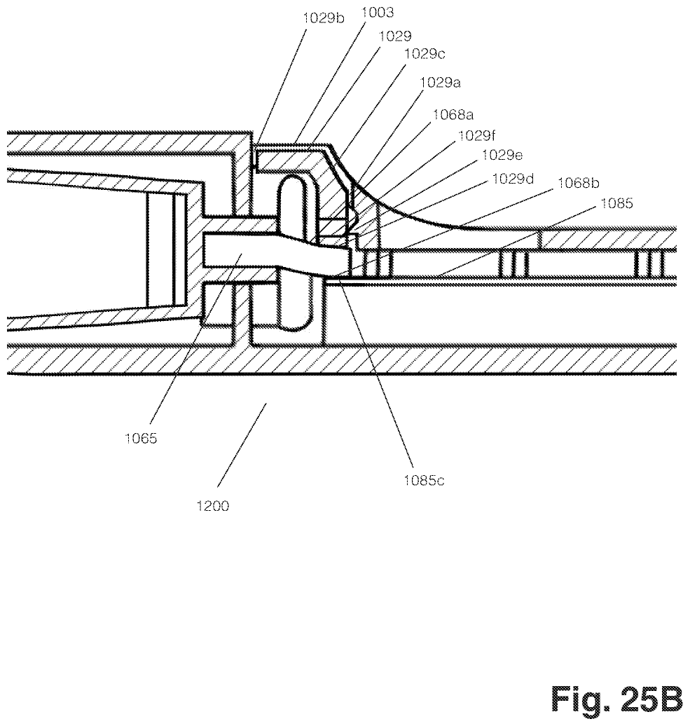

Test device 1000 has many of the same features of test device 100 except that the transfer pad is separated by a distance from test strip and the distance is closed by operator-controlled depression of a button disposed in test device housing anterior member. The space between the transfer pad and the test strip does not permit significant fluid transmission (e.g., does not permit fluid transmission) from the transfer pad to the test strip until the button is depressed, which closes the space and establishes a transfer pad-test strip interface. The button provides for operator control over the amount of time that sample fluid is retained in the transfer pad before sample fluid in the transfer pad is permitted to transmit from the transfer pad to the test strip, which can allow sufficient time for reagents in the transfer pad to interact with sample fluid analyte(s). As shown in FIG. 23 to FIG. 25B, test device 1000 includes test device housing posterior member 1002, test device housing anterior member 1003, test device housing base extension 1007 and test device housing window 1010, each in the same orientation as counterparts in test device 100. Test device 1000 also includes test device cartridge housing 1070, cartridge terminal projection 1074, transfer pad 1065, first cartridge housing pad 1045 and second cartridge housing pad 1046. Transfer pad 1065 includes anterior surface 1068a and posterior surface 1068b, where posterior surface of 1068b of transfer pad 1065 is spaced a distance from anterior surface 1085c of test strip 1085, as shown in FIG. 25A. A composition and/or kit provided to/by an operator typically includes test device 1000 in the configuration shown in FIG. 25A. After sample collection device 1100 containing sample fluid has been inserted in test device 1000 for a period of time, an operator can depress test device housing button 1029 to deform transfer pad 1065 in the anterior to posterior direction, thereby establishing a transfer pad-test strip interface as illustrated in FIG. 25B and transmission of fluid in the proximal to distal direction from transfer pad 1065 to test strip 1085. Test device housing anterior member 1003 includes button perimeter slits 1029a, 1029b and 1029c that in part define the perimeter of button 1029. A portion of test device housing anterior member 1003 disposed at a terminus of each of slits 1029a and 1029b and opposed to slit 1029c functions as a living hinge when an operator depresses button 1029. Disposed at a posterior portion of button 1029 is extension bar 1029g that includes posterior terminus 1029d. Button posterior terminus 1029d contacts a portion of anterior surface 1068a, and depression of button 1029 causes posterior terminus 1029d to exert a force in the anterior to posterior direction to anterior surface 1068a of transfer pad 1065, thereby deforming a portion of transfer pad 1065 as shown in FIG. 25B. Test device housing anterior member 1003 includes button catch 1029e and extension bar 1029g includes button catch counterpart 1029f adjacent button catch 1029e. Button catch counterpart 1029f can be a depression in extension bar 1029g in which button catch 1029e can reside after depression of button 1029, or can be a projection against which button catch 1029e is disposed after depression of button 1029. Button catch 1029e is anteriorly-disposed relative to button catch counterpart 1029f when button 1029 is not depressed (see FIG. 25A). Button catch 1029e is placed in connection with button catch counterpart 1029f after button 1029 has been depressed, thereby retaining button 1029 and extension bar 1029g in the depressed position shown in FIG. 25B, which maintains the transfer pad-test strip interface and permits continuous transmission of fluid from transfer pad 1065 to test strip 1085.

### Compositions, Kits and Assemblies Containing Sample Collection and Test Device Components

Provided in certain aspects is a composition or kit that includes: a sample collection device component described herein and a test device component described herein, where the sample collection device is separate from the test device and is not joined or connected to the test device. The sample collection device is configured to join to the test device but the sample collection device is not joined and is physically separated from the test device in the composition or kit. A composition or kit can include any suitable number of separate containers or compartments, and in certain implementations, the sample collection device and the test device are in separate compartments and/or containers. A container can be any suitable container for storing a test device or sample collection device for a period of time, including for example rigid, semi-rigid or flexible containers (e.g., a flexible bag that optionally can be sealed).

A kit or composition can include one or more reagents described herein, and in certain implementations, contains a reader device suitable for detecting presence, absence and/or amount of a detectable signal emitted from a test device lateral flow strip through a test device housing window. A kit or composition can include instructions for carrying out an assembly process or method of using a sample collection device and/or test device as described herein. Instructions and/or descriptions may be in tangible form (e.g., paper and the like) or electronic form (e.g., computer readable file on a tangle medium (e.g., memory device)) and may be included in a kit insert. A kit may include a written description of an internet location that provides such instructions or descriptions.

The sample pad of the sample collection device component generally is compressible as described herein. In certain composition and kit implementations, the sample collection device handle distal terminus is configured to contact the test device housing proximal terminus for when the test device and sample collection device are joined (e.g., connected, sealingly connected, in locked connection). In certain composition and kit implementations, a lateral distance between the sample collection device handle distal terminus and the sample pad distal terminus is a first lateral distance; a lateral distance (i) between the test device housing proximal terminus and the interior surface of the cartridge housing terminal wall, or (ii) between the test device housing proximal terminus and a proximal surface of a cartridge housing pad where the cartridge housing contains a cartridge housing pad, is a second lateral distance; and the first lateral distance is greater than the second lateral distance.

In certain aspects, provided is an assembly that includes a sample collection device component described herein and a test device component described herein, where the sample collection device is joined with the test device (e.g., connected, sealingly connected, in locked connection). The test device generally is distally disposed from the sample collection device handle, and the sample pad and at least a portion of the stem of the sample collection device often are contained within the test device. The sample pad and at least a portion of the stem of the sample collection device often are disposed within the interior of the cartridge housing of the test device.

In certain assembly implementations, the distal terminus of the sample pad contacts at least a portion of the interior surface of the cartridge housing terminal wall when there is no optional cartridge housing pad present in the test device. In certain assembly implementations, the distal terminus of the sample pad contacts at least a portion of a proximal surface of a cartridge housing pad when one or more such pads are present in the test device.

In assembly implementations, the sample pad generally is laterally compressed in the assembly relative to the lateral length of the sample pad when the sample collection device and the test device are separate and not joined. In certain instances, the lateral length between the sample pad proximal terminus and the sample pad distal terminus in the sample collection device not joined to the test device is a first lateral distance; a lateral length between the sample pad proximal terminus and the sample pad distal terminus in the sample collection device joined to the test device is a second lateral distance; and the first lateral distance is greater than the second lateral distance. In an assembly, the second lateral distance sometimes is about 50% to about 90% of the first lateral distance, or about 60% to about 85% of the first lateral distance, or about 70% to about 80% of the first lateral distance, or about 50%, 55%, 60%, 65%, 70%, 75%, 80%, 85%, 90% or 95% of the first lateral distance. The sample pad generally is compressed when the sample collection device and the test device are connected, for example in sealing connection and/or in locked connection.

In an assembly, the sample pad often is a single pad, where the distal terminus of the sample pad directly contacts the interior surface of the cartridge housing terminal wall or portion thereof or directly contacts the proximal surface of a cartridge housing pad or portion thereof. In an assembly, (i) there is a midpoint between the sidewall exterior surfaces at the distal terminus of the sample pad, (ii) there is a midpoint between the sidewall exterior surfaces at the proximal terminus of the cartridge housing pad, and (iii) there is a midpoint between the exterior surfaces at the proximal terminus of the transfer pad. In certain assembly implementations, the midpoint of (i) is concentric with the midpoint of (ii); or the midpoint of (ii) is concentric with the midpoint of (iii); or the midpoint of (i), the midpoint of (ii) and the midpoint of (iii) are concentric. The latter orientations can permit linear sample fluid transfer from the sample pad to the optional cartridge housing pad to the transfer pad.

In certain assembly implementations, the sample pad or portion thereof resides within the interior (lumen) of a cartridge housing member of the test device. In an assembly, the sample pad in a compressed state often is deformed, and sometimes includes bent and/or swollen portions. An effective outer diameter of a compressed sample pad is defined as a minimum cross-sectional diameter that fits the entire cross-section of a deformed sample pad from the proximal terminus to the distal terminus of the sample pad. In certain assembly implementations, the minimum interior transverse diameter of a cartridge housing lumen portion in which the sample pad resides, is equal to or greater than the sample pad effective outer diameter. The minimum interior transverse diameter of the cartridge housing lumen portion in which the sample pad resides sometimes is 0.01% to 50% greater than the sample pad effective outer diameter. The difference between the minimum interior transverse diameter of the cartridge housing lumen portion in which the sample pad resides and the sample pad effective outer diameter may be the same as illustrated in FIG. 15, for example, or different (e.g., the difference may be less).

In certain assembly implementations, the sample collection device is in connection with the test device (e.g., sealing connection, locked connection). A connection sometimes is reversible and sometimes is irreversible (i.e., locked connection) under a tool-less lateral pulling force generated by a human of average strength. In certain instances, the sample collection device includes a connector member disposed on the stem; the test device includes a connector member counterpart disposed on an interior surface of the test device; and the connector member of the sample collection device is connected to the connector member counterpart of the test device. In certain instances, the sample collection device includes a connector member disposed in the handle; the test device includes a connector member counterpart disposed on an exterior surface of the test device; and the connector member of the sample collection device is connected to the connector member counterpart of the test device. The connector member of the sample collection device sometimes is an annular projection, and the connector member counterpart of the test device sometimes is an annular groove, where the annular projection is disposed in the annual groove. The connector member of the sample collection device sometimes includes a threaded member, and sometimes the connector member counterpart of the test device includes a threaded member that engages the threaded member of the sample collection device. When present, a threaded member of a sample collection device sometimes is disposed on an interior surface of the handle and a threaded member of the test device sometimes is disposed on a hollow shank extending from the cartridge and from the proximal terminus of the test device housing.

In certain assembly implementations, the sample collection device is in sealing engagement and/or in sealing connection with the test device. A sealing member sometimes is disposed at a stem of a sample collection device, where the sealing member sometimes is in sealing contact with (i) a sealing member counterpart disposed at an interior wall surface of the test device, or (ii) an interior wall surface of the test device.

In certain assembly implementations, a sample pad of a sample collection device includes sample fluid, as described herein. Sample fluid typically transmits from the sample pad of the sample collection device to the transfer pad in the test device and from the transfer pad to the test strip in the test device. An assembly often includes a sample expression zone that contains a sample pad compression zone, a sample transfer zone distally-disposed relative to the sample expression zone, and a sample readout zone distally-disposed relative to the sample expression zone. Sample fluid often transmits from the sample pad in the sample compression zone, often transmits to and from the transfer pad in the sample transfer zone, and transmits through the test strip in the sample readout zone. In the sample expression zone, sample fluid typically transmits from the sample pad of the sample collection device through one or more bores disposed in the terminal wall of the cartridge housing, and sample fluid then often transmits to the transfer pad in the sample transfer zone. In the sample expression zone, sample fluid sometimes transmits from the sample pad of the sample collection device, through one or more cartridge housing pads, then through one or more bores disposed in the terminal wall of the cartridge housing, and then to the transfer pad.

Without being limited by theory, compression of the sample pad generates a back pressure that expresses fluid from the sample pad and motivates transmission of the expressed sample fluid through the test device, from the sample pad to one or more cartridge housing pads when present in a test device, to the transfer pad through one or more bores in the terminal wall of the cartridge housing, and from the transfer pad to the test strip. Without being limited by theory, compression of the sample pad functions as a virtual plunger that motivates sample fluid to express from the sample pad and transmit laterally in the proximal to distal direction through the test device.

Also without being limited by theory, metering of sample fluid in the test device generally is governed in part (i) by the size, porosity and retention volume of the sample pad of the sample collection device, and (ii) by the size, porosity and retention volume of the transfer pad of the test device. The retention volume of the transfer pad generally serves to meter the amount of fluid transmitted to the test strip. The retention volume of the of the transfer pad generally is less than the volume capacity of the sample pad, accounting for sample fluid retained by the sample pad and any cartridge housing pad(s).

Without being limited by theory, in a single step of joining the sample collection device component to the test device component (e.g., in sealing and/or locked connection), sample fluid is expressed from the sample pad, transmitted laterally in the proximal to distal direction through the test device in an amount metered by the sample pad to the one or more optional cartridge housing pads and to the transfer pad, and in an amount metered by the transfer pad to the test strip. Metering of sample fluid volume within the test device without requiring action by an operator other than joining the sample collection device component to the test device component advantageously provides for controlling the ratio of sample fluid volume to reagents and other components in pads and/or the test strip, thereby significantly reducing variance of test results caused by operator error and increasing ease of use.

Without being limited by theory, in a single step of joining the sample collection device component to the test device component (e.g., in sealing and/or locked connection), sample fluid is processed in the test device prior to transmission to the test strip. Such processing sometimes includes retaining certain sample fluid components in the sample pad and/or optional cartridge housing pad(s) (e.g. retaining mucins (e.g., high molecular weight mucins and/or low molecular weight mucins) and/or contacting sample fluid with one or more reagents in the sample pad, optional cartridge housing pad(s) and/or transfer pad. With a single step of joining the sample collection device component to the test device component (e.g., in sealing and/or locked connection), sample fluid is processed in the test device without prior sample fluid processing by an operator, thereby significantly reducing variance of test results caused by operator error and enhancing ease of use. For example, a single step of joining the sample collection device component to the test device component allows for integration of sample fluid processing steps within the test device, thereby obviating or reducing the number of prior processing steps such as prior freezing or thawing of sample fluid, prior filtration of sample fluid (i.e., unprocessed, native sample can be collected on the sample pad and introduced to the test device) and/or prior contact of sample fluid with one or more reagents, for example.

Without being limited by theory, rate of fluid flow from the sample pad through the test device is in part governed by (i) the degree of sample pad compression, (ii) the surface area of bores in the cartridge housing terminal wall, (iii) porosity of the transfer pad, and (iv) fluid transfer rate of the test strip. A higher degree of lateral sample pad compression can generate a higher amount of back pressure and a higher flow rate of sample fluid from the sample pad, relative to a flow rate associated with a lower degree of lateral sample pad compression. Bore size can be expressed in terms of a ratio between the overall bore surface area (e.g., combined surface area of multiple bores) to the surface area of the interior surface of the terminal wall of the cartridge housing that contains the bore(s), as described herein. A higher ratio can result in a lower sample fluid flow rate as compared to a flow rate associated with a relatively lower ratio. A higher transfer pad porosity can result in a higher sample fluid flow rate compared to a flow rate associated with a lower transfer pad porosity.

The degree of sample pad compression, bore surface area in the cartridge housing terminal wall, and transfer pad pore size typically are optimized for (i) efficient sample fluid loading into the transfer pad, and (ii) sufficient time for reagents present in pads and/or on the strip to interact with sample fluid prior to sample fluid migration to a detection zone on the strip in the test device, for example. The rate of sample fluid flowing through an assembly also can be optimized by inclusion of one or more fluid flow rate modulating agents in one or more pads of a device described herein. For example, a water-absorbing agent can be included in one or more pads that can reduce a fluid flow rate. Without being limited by theory, a water-absorbing agent can absorb water when sample fluid contacts the agent and the act of water absorption can reduce flow of the sample fluid. A water-absorbing agent can be included in a portion of a transfer pad in certain implementations (e.g., a water-absorbing agent can be included in a portion of transfer pad disposed adjacent to the distal terminus of the pad).

Efficient sample fluid loading into the transfer pad generally results in sample fluid expressed from the sample pad absorbing into the transfer pad at a rate that (i) maximizes the amount of the sample fluid transferred from the transfer pad to the strip, and (ii) eliminates or reduces the amount of fluid exiting the transfer pad into a portion of the test device interior not in the strip (referred to herein as residual sample fluid). Extension structures in the test device housing (e.g., extension wall, extension bar), as described herein, can together serve as a containment structure, and thereby a reservoir, for any residual sample fluid. For example, when present in the test device housing, the first extension wall, second extension wall, third extension wall, fourth extension wall, extension bar, integrated posts and interior window projection(s) can collectively serve as a reservoir that can contain any residual sample fluid, and the residual sample fluid can be reabsorbed into the transfer pad if any initially exits the transfer pad. The residual sample fluid can be reabsorbed into the transfer pad by changing the orientation of the test device (e.g., an operator may rotate or shake the device or stand the device in an orientation in which the device does not rest on a base extension).

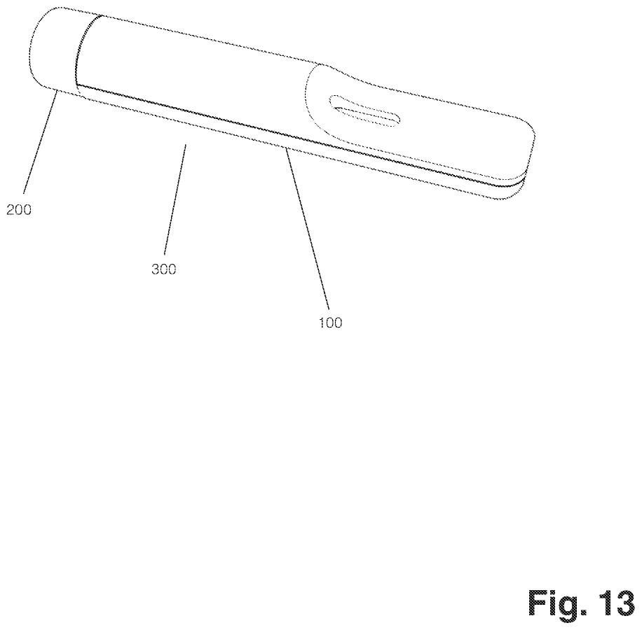

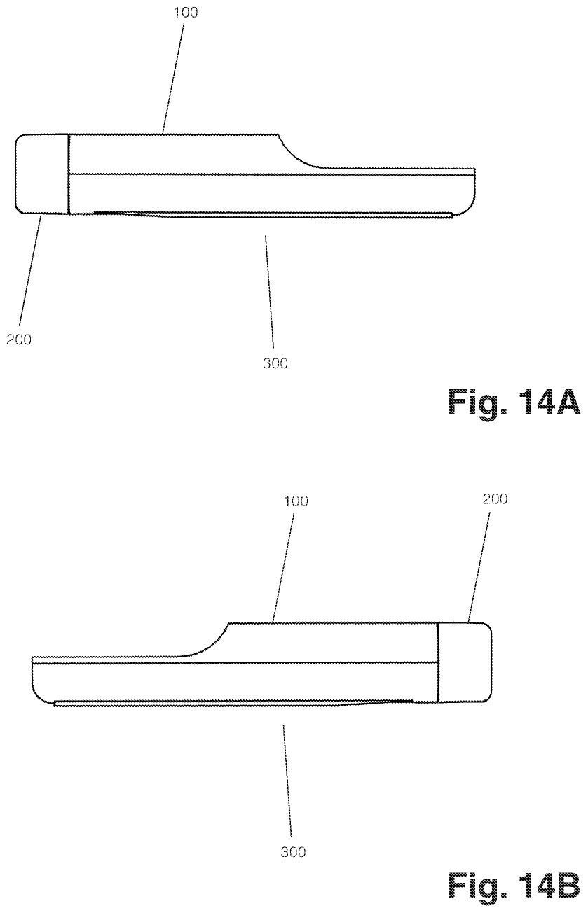

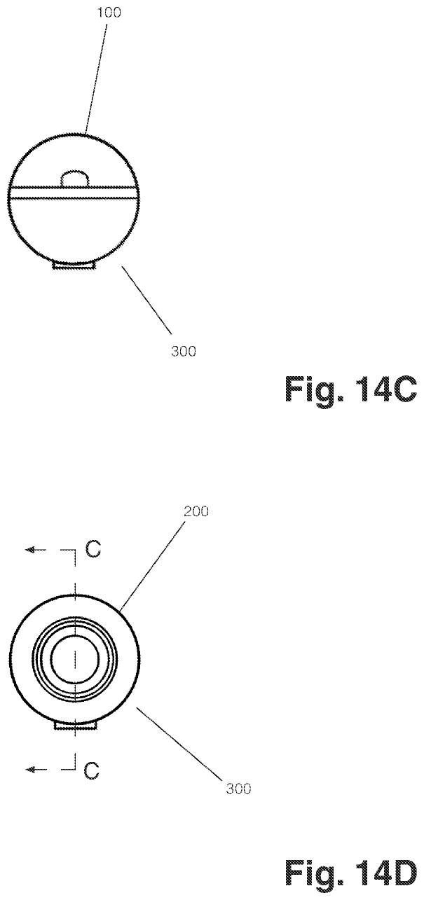

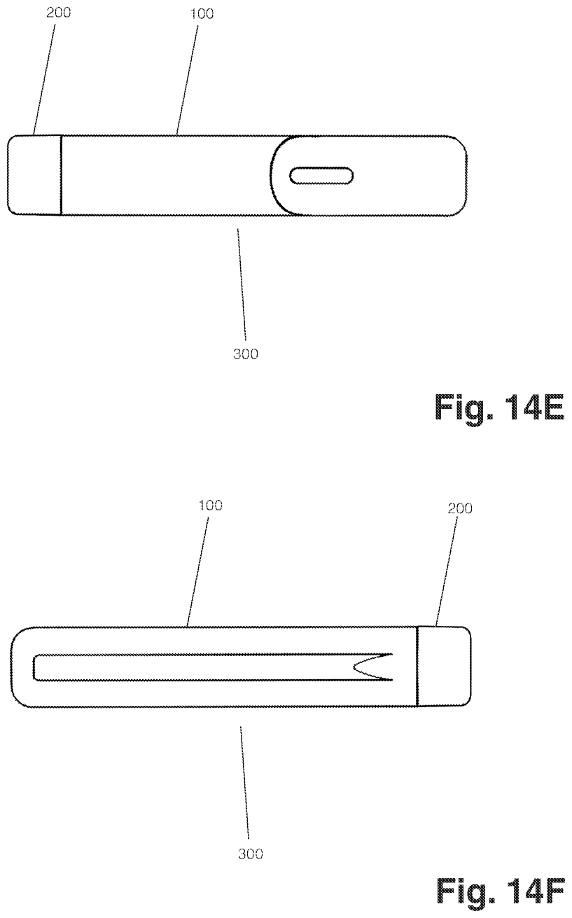

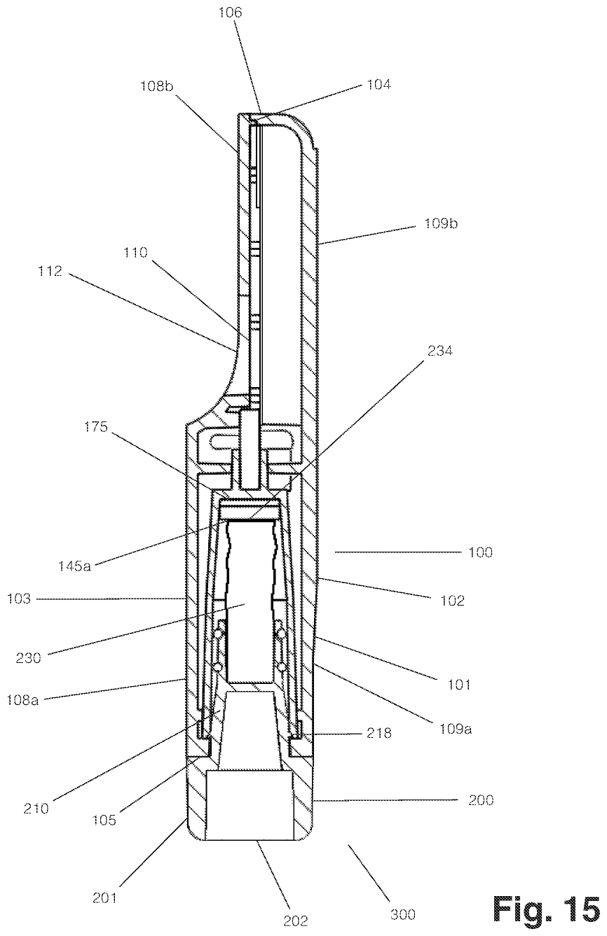

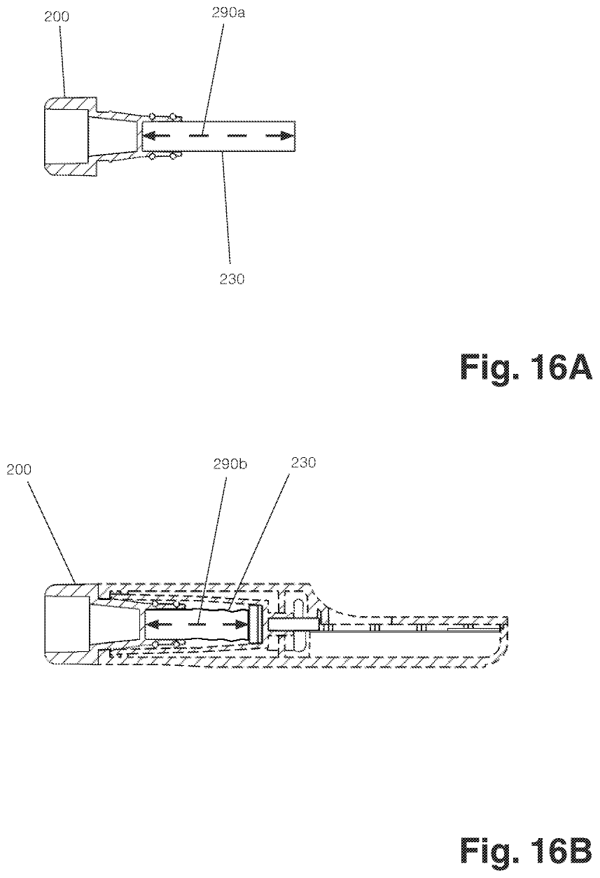

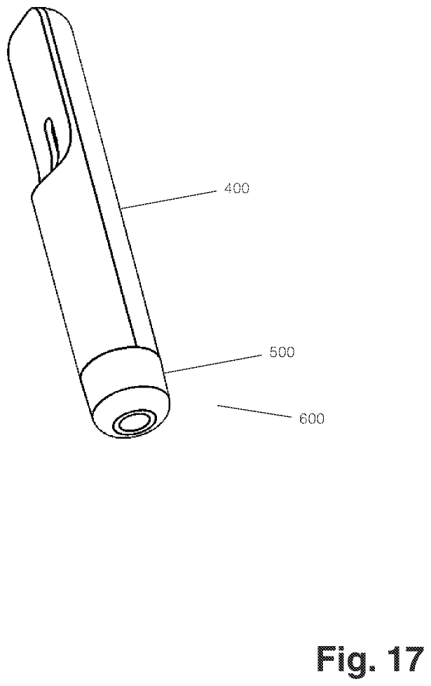

A non-limiting example of an assembly is assembly 300 that includes test device 100 and sample collection device 200, as illustrated in FIG. 13 to FIG. 16B. Illustrated in FIG. 15 is assembly 300 with sample pad 230 in a compressed state relative to the relaxed state of sample pad 230 shown in FIG. 16A where sample collection device 200 is not joined to test device 100. Compression of sample pad 230 results from distal surface 234 of sample pad 230 contacting proximal surface 145a of cartridge housing pad 145, which is backed indirectly by rigid cartridge housing terminal wall 175, in assembly 300 shown in FIG. 15. In the relaxed state, lateral length 290a of sample pad 230 is illustrated in FIG. 16A when the sample collection device not joined to the test device, and lateral length 290b of the sample pad is illustrated in FIG. 16B when the sample collection device is joined to the test device. Lateral length 290b in assembly 300 is shorter than lateral length 290a. Joining sample collection device 200 to test device 100 is completed by connection of stem annular protrusion 218 (connector member) of sample collection device 200 with annular groove 178c of test device 100 (connector member counterpart). While lateral length 290b in assembly 300 is about 75% of lateral length 290a of separate sample collection device 200, lateral length 290b can be about 50% to about 90% of lateral length 290a, or about 60% to about 85% of lateral length 290a, or about 70% to about 80% of lateral length 290a.

As shown in FIG. 10, test device 100 includes sample expression zone 130 (containing a compression zone), sample transfer zone 150 and sample readout zone 180. In sample expression zone 130 of assembly 300, sample fluid is expressed in the proximal to distal direction as a result of compression of sample pad 230. Also in sample expression zone 130 of assembly 300, sample fluid expressed from sample pad 230 transmits in the proximal to distal direction to first cartridge housing pad 145, then to second cartridge housing pad 146, and then through bores 177a and 177b of cartridge housing terminal wall 175 to transfer pad 165. In sample transfer zone 150 of assembly 300, sample fluid transmitted to transfer pad 165 transmits to test strip 185, via posterior surface 168b of transfer pad 165 in contact with anterior surface 185c of test strip 185 at the transfer pad-test strip interface. Sample fluid transmits through test strip 185 in the proximal to distal direction through one or more reaction zones in the test strip, and a detectable signal sometimes is emitted in sample readout zone 180 at a location disposed opposite window 110 depending on whether or not an analyte is present in the sample fluid.

### Methods of Use

Provided in certain aspects is a method for preparing an assembly that includes joining a sample collection device described herein to a test device component described herein, thereby forming the assembly. The joining generally includes inserting the collection device into the interior of the cartridge housing, and often includes inserting in a lateral direction and in the proximal to distal direction, a portion of the sample collection device component containing at least a portion of the sample pad into the interior of the cartridge housing the test device component. The stem or portion thereof of the sample collection device component often is inserted into the interior of the cartridge housing. An operator typically grasps the handle of the sample collection device component, after sample fluid is absorbed into the sample pad, and inserts in a lateral motion, in the proximal to distal direction, a portion of the sample collection device component containing at least a portion of the sample pad through the test device housing proximal opening, and into the interior of the cartridge housing, of the test device component. Inserting a portion of a sample collection device in the lateral direction and in the proximal to distal direction sometimes is a slidable insertion, and sometimes includes twisting or rotation of the sample collection device around a virtual central lateral axis (i.e., around a lateral axis disposed in the center of the sample collection device and parallel to lateral axis 198 shown in FIG. 10). Sometimes slidable insertion of the sample collection device does not include twisting or rotation of the sample collection device around a virtual central lateral axis.

The sample pad of the sample collection component device that is joined to the test device component often contains sample fluid. The sample pad of the sample collection device component generally is contacted with sample fluid under conditions in which the sample pad absorbs sample fluid prior to joining the sample collection device component to the test device component. Prior to joining the sample collection device component to the test device component, a property of the sample pad of the sample collection device sometimes changes upon contact with the sample fluid, indicating to an operator that a sufficient amount of sample fluid has been absorbed into the sample pad. After a change is detected or observed in such implementations, a sample collection device sometimes is joined to a test device without further manipulation of the sample collection device, or After a change is detected or observed in such implementations, a sample collection device sometimes is placed in a sealable container (e.g., rigid container, a flexible bag), the container is sealed, and then the collection device is later removed from the container and joined to a test device.

Joining of the sample collection device component to the test device component often is completed when the sample collection device component is connected to the test device component. Joining of the sample collection device component to the test device component often is completed when the sample collection device component is sealingly connected to the test device component. Joining of the sample collection device component to the test device component often is completed when the sample collection device component is in locked connection with the test device component, where joining the sample collection device to the test device includes engaging a connector member of the sample collection device with a connector member counterpart of the test device. As described herein, a connector member and a connector member counterpart sometimes are engaged by an interference fit or sometimes by a threaded fit, and sometimes the connection is reversible or is a locked connection. In certain implementations, engaging the connector member and the connector member counterpart emits an audible sound, indicating to an operator that the members are connected and joining is complete.

After the sample collection device component has been joined to the test device component (e.g., connected, sealingly connected, in locked connection, completely joined), the sample pad of the sample collection device generally is laterally compressed within the cartridge housing of the test device. After the sample collection device component has been joined to the test device component (e.g., connected, sealingly connected, in locked connection, completely joined), sample fluid contained in the sample pad is expressed from the sample pad. Without being limited by theory, compression of the sample pad generates a back pressure that expresses fluid from the sample pad in a sample expression zone of the test device and motivates transmission of the expressed sample fluid through the test device, as described herein.

In certain implementations, the sample pad retains components in the sample fluid, and sample fluid expressed from the sample pad includes an amount of the components less than the amount in the sample fluid applied to the sample pad and absorbed in the sample pad prior to expression of sample fluid from the sample pad in the test device component. In certain instances, the sample pad retains high molecular weight mucins and the sample fluid expressed from the sample pad includes an amount of the high molecular weight mucins lower than the amount in the sample fluid applied to the sample pad. High molecular weight mucins generally have a molecular weight of 500 kiloDaltons or greater. Without being limited by theory, reducing mucin content in sample fluid transmitted to the test strip and/or transfer pad advantageously reduces sample fluid viscosity.

After sample fluid is expressed from the sample pad of the sample collection device component in a sample expression zone, sample fluid transmits laterally in the proximal to distal direction from the sample pad to a transfer pad in the test device component, and from the transfer pad to a test strip in the test device. The sample fluid typically transmits from the sample pad of the sample collection device through one or more bores disposed in the terminal wall of the cartridge housing, to the transfer pad distally-disposed with respect to the terminal wall. In certain instances, sample fluid expressed by the sample pad of the sample collection device transmits through one or more cartridge housing pads, then through one or more bores disposed in the terminal wall of the cartridge housing, then to the transfer pad and then to the test strip. In certain implementations, sample fluid transmits laterally in the proximal to distal direction from the sample pad to the one or more cartridge housing pads and then to the transfer pad, and then transmits longitudinally in the anterior to posterior direction from the transfer pad to a proximal region of the test strip disposed at a transfer pad-test strip interface. In certain implementations, sample fluid transmits from the sample pad, after the sample collection device is joined (e.g., connected, sealingly connected, in locked connection, completely joined), to the test device (i.e., time zero), to the sample readout zone (e.g., on the test strip opposite the window of the test device housing) in about 1 minute to about 30 minutes depending on the assay(s) conducted by the test device.

Generally, input sample fluid enters a particular pad in the test device component and output sample fluid is transmitted (i.e., outputted) from the pad. When present in a cartridge housing of a test device component, at least one of the one or more optional cartridge housing pads sometimes retains one or more components in the input sample fluid, and sample fluid outputted from the at least one of the one or more cartridge housing pads includes an amount of the one or more components less than the amount in the input sample fluid. In certain instances, the at least one of the one or more optional cartridge housing pads retains low molecular weight mucins and the sample fluid outputted by the optional pads includes an amount of the low molecular weight mucins lower than the amount of the low molecular weight mucins in the input sample fluid. Low molecular weight mucins have a molecular weight of 300 kiloDaltons or less. Without being limited by theory, reducing mucin content in sample fluid transmitted to the test strip and/or transfer pad advantageously reduces sample fluid viscosity.

In certain implementations, one or more of the pads of the sample collection device and the test device (i.e., one or more of the sample pad, transfer pad and one or more of the optional cartridge housing pads) and/or the test strip of the test device, includes one or more reagents that interact with the sample fluid (e.g., bind to an analyte in the sample fluid). Sample fluid often is aqueous. In certain instances, the one or more reagents are in dry form or semi-hydrated form and interact in solution with the sample fluid when the sample fluid enters the pad. In certain instances, at least a portion of the one or more reagents dissolves into the input sample fluid in or on the pad or test strip. Without being limited by theory, as sample fluid hydrates and transmits through a pad and/or test strip, one or more dry or semi-hydrated reagents present hydrate and mix with the sample fluid by turbulence.

Sample fluid generally transmits from a portion of the transfer pad to a portion of the test strip. In certain implementations, sample fluid transmits from a portion of a posterior surface of the transfer pad to a portion of an anterior surface of the test strip opposite the portion of the transfer pad (e.g., at a transfer pad-test strip interface). The portion of the transfer pad and the portion of the transfer strip sometimes are in direct contact prior to joining the sample collection device to the test device. In a non-limiting example, the distally-disposed portion of posterior surface 168b of transfer pad 165 contacts a proximally-disposed portion of anterior surface 185c of test strip 185, at a transfer pad-test strip interface, prior to and after sample collection device component 200 is joined to test device component 100, in assembly 300 shown in FIG. 13 to FIG. 16B.

In certain instances, the portion of the transfer pad and the portion of the test strip are not in direct contact prior to, and after, joining the sample collection device to the test device, and the portion of the transfer pad and the portion of the test strip are placed in direct contact after the sample collection device is joined to the test device. The portion of the transfer pad and the portion of the test strip sometimes are placed in direct contact after an amount of time elapses after the sample collection device is joined to the test device. In certain instances, the portion of the transfer pad and the portion of the test strip are separated by a removable barrier (e.g., a pull tab described herein) prior to, and after, joining the sample collection device to the test device, and the portion of the transfer pad and the portion of the test strip are placed in direct contact after the sample collection device is joined to the test device by removing the portion of the removable barrier disposed at the transfer pad-test strip interface. An operator can remove the portion of the removable barrier (e.g., pull tab described herein) disposed between the portion of the transfer pad and the portion of the test strip after a period of time elapses from the time the sample collection device is joined to the test device component (i.e., a time period that elapses from time zero described herein). The removable barrier (e.g., pull tab described herein) sometimes is retained in the test device component after a portion of it is removed from between the portion of the transfer pad and the portion of the test strip, and sometimes the removable barrier is separated from the test device component.

In a non-limiting example, the distally-disposed portion of posterior surface 768b of transfer pad 765 is separated from a proximally-disposed portion of anterior surface 785c of test strip 785, by a portion of removable barrier pull tab 790 disposed at the interface, prior to and after sample collection device component 800 is joined to test device component 700, in assembly 900 shown in FIG. 21A to FIG. 22. After a period of time has elapsed from the time sample collection device component 800 has been joined to test device component 700 (e.g., a period of time from time zero described herein), the distally-disposed portion of posterior surface 768b of transfer pad 765 is placed in contact with the proximally-disposed portion of anterior surface 785c of test strip 785, at the transfer pad-test strip interface, by removing the portion of barrier pull tab 790 from the interface. A portion of barrier pull tab 790 may be disposed in device component 700 after the portion of barrier pull tab 790 that was disposed at the interface has been displaced from the interface, or barrier pull tab 790 may be separated from device component 700 after the portion of barrier pull tab 790 that was disposed at the interface has been displaced from the interface.

In certain implementations, the portion of the transfer pad and the portion of the test strip are separated prior to, and after, joining the sample collection device to the test device, and the portion of the transfer pad and the portion of the test strip are placed in direct contact after the sample collection device is joined to the test device by operating a button present in the test device housing. In certain instances, the button is depressed in the anterior to posterior direction, and a portion of the device housing in contact with, or placed in contact with, an anterior surface of the transfer pad opposite the portion of the test strip after the button is depressed causes the portion of the transfer pad to contact the portion of the test strip. The portion of the device housing in contact with, or placed in contact with, the anterior surface of the transfer pad sometimes is an extension bar extending from an interior surface of an anterior portion of the test device housing sidewall. An operator can depress the button after a period of time elapses from the time the sample collection device is joined to the test device component (i.e., a time period that elapses from time zero described herein). An operator sometimes depresses the button for a sustained period of time. Sometimes an operator depresses the button and a catch in the test device component retains the button in a depressed configuration after an operator has depressed the button.

In a non-limiting example, the distally-disposed portion of posterior surface 1068b of transfer pad 1065 is separated from a proximally-disposed portion of anterior surface 1085c of test strip 1085 prior to and after sample collection device component 1100 is joined to test device component 1000, in assembly 1200 shown in FIG. 23 to FIG. 25B. After a period of time has elapsed from the time sample collection device component 1100 has been joined to test device component 1000 (e.g., a period of time from time zero described herein), the distally-disposed portion of posterior surface 1068b of transfer pad 1065 is placed in contact with the proximally-disposed portion of anterior surface 1085c of test strip 1085, at a transfer pad-test strip interface, by depressing button 1029 of test device housing anterior member 1003. Depressing button 1029 about a living hinge defined by slits 1029a, 1029b and 1029c, places posterior terminus 1029d of test device housing extension bar 1029g in contact with anterior surface 1068a of transfer pad 1065, which deflects a distally-disposed portion of transfer pad 1065 longitudinally, in the anterior to posterior direction, thereby placing the distally-disposed portion of posterior surface 1068b of transfer pad 1065 in contact with the proximally-disposed portion of anterior surface 1085c of test strip 1085 (e.g., illustrated in FIG. 25A and FIG. 25B). After button 1029 is depressed, button 1029 and extension bar 1029g are retained in the depressed position by interaction of catch 1029e disposed adjacent to extension bar 1029g with catch counterpart 1029f disposed in or on extension bar 1029g (e.g., illustrated in FIG. 25A and FIG. 25B).

In certain aspects, provided is a method for determining presence or absence and/or amount of an analyte in a sample that includes: detecting a detectable signal from a lateral flow strip through a window of a test device component of an assembly described herein, or an assembly prepared by a method described herein, and determining the presence or absence and/or amount of the analyte in the sample from the detectable signal. The detectable signal often is an optically detectable signal, which can be detected by an instrument or by eye. An optical signal can be detected with or without illumination (e.g., with or without dedicated illumination). Sometimes a signal detected optically is a signal emitted by nanoparticles (e.g., gold, latex, silver or carbon nanoparticles). An optically detectable signal sometimes is emitted by a fluorescent probe excited by a light source (e.g., laser or light-emitting diode (LED) source) and a corresponding filter set to detect light emitted by the probe. In a specific implementation, R-phycoerythrin is detected after excitation by a laser at 515 nanometers (nm) and using a 10 nm band-pass filter at 580 nm. An optical signal can be detected by a human eye (e.g., by eye of an operator (e.g., human operator)) and can be augmented (e.g., by a smartphone). In certain instances, an optical signal can be detected using a reader device that includes one or more of a complementary metal oxide semiconductor (CMOS), charge-coupled device (CCD) and/or photodiode sensor, for example, where internal illumination is dependent on the detectable label being detected. Presence, absence and/or amount of an analyte can be determined for a sample according to presence, absence and/or amount of a detectable signal detected through the window of a device assembly by any suitable process known in the art (e.g., consulting instructions included with or referred to by a kit, consulting a standard curve, consulting a look-up table).

An overview of methodology for using sample collection device and test device components is provided hereafter.

Sample Collection—The user collecting the sample grasps the Sample Collector (e.g., sample collection device 200) by the Sample Collector Handle (e.g., handle 201). The Absorbent Collection Pad (e.g., sample pad 230) end of the Sample Collector is placed into or onto the area (including but not limited to mouth, container filled with liquid to be tested, pool of liquid not held in a container, etc.) where the sample will be absorbed into the absorbent material. The absorbent material may contain chemistry including but not limited to buffers, indicators, surfactants, secretion enhancers that dissolve and mix with the sample being collected. The Sample Collector may take different forms including, but not limited to a swab stick, bladder, different shaped absorbent pads with various shapes and forms of handles, a cup, beaker, syringe, or pipette. The Sample Collector may have a sufficiency indictor (either physical or chemical) imbedded in or in a pad in contact with the Absorbent Collection Pad to identify when adequate volume has been absorbed. Once the sample reaches the Indicator Reaction Zone (e.g., between sealing members 215 and 217 in sample collection device 200), the transition of the indicator takes place, alerting the user that sufficient volume was collected. The sufficiency indicator can be visualized through a window within the sample collector, by a physical or mechanical conformational shift within the sample collection device. At this point the sample collector device 200 is ready for insertion in test device 100, as shown in FIG. 1.

Sample Delivery—The user inserts the Absorbent Collection Pad (e.g., sample pad 230) into the Sample Collector Port (e.g., proximal opening 120) of the Device Housing (e.g., test device housing 101). The Sample Collector Handle (e.g., handle 201) is pushed until it stops. One potential indicator for correct seating of the Sample Collector (e.g., sample collection device 200) could be perceived through a physical change or an audible click generated by the Click Lock Feature (e.g., annular protrusion 218 of sample collection device 200) coming to rest in the locked position. This feature also assures that the user will not reuse the device because the sample collector (e.g., sample collection device 200) can no longer be pulled out of the device housing (e.g., test device housing 101). The motion of insertion could be either sliding or screwing into the Sample Collection Port (e.g., proximal opening 120). The Sample Port (e.g., proximal opening 120) leads to a Sample Process Cartridge (e.g., cartridge housing lumen 173d, 173e) which contains one or a series of Pads (e.g., cartridge housing pads 145 and 146), a Restriction Structure (e.g., terminal wall 175) and Sample Transport Ports (e.g., bores 177a, 177b). The port(s) may consist of a single or multiple open zones for transfer of sample. The compression action causes the Absorbent Collection Pad (e.g., sample pad 230) to compress at the Compression Zone (e.g., in sample expression zone 130) due to the presence of the Restriction Structure (e.g., terminal wall 175). This releases a metered volume of sample based on the dimensions of the Absorbent Collection Pad (e.g., sample pad 230), the Sample Process Cartridge Housing (e.g., cartridge housing 170) and the Restriction Structure (e.g., terminal wall 175). The sample flows through the Filtration/Conjugate/Chemistry Pads (e.g., cartridge housing pads 145 and 146), causing the sample to dissolve the chemistry associated with the pad(s). FIG. 5 and FIG. 11 show two pads, but additional pads may be included to add support for the structural support of the other pads or to add additional chemistry or to enhance filtration. The filter can be made of a porous pad, or other filtering material, substance or any other means by which a sample may be filtered. The filter may contain materials or chemistry to separate portions of the sample and pre-conditioning materials including but not limited to surfactants, buffers, indicators, etc. The resulting mixture of sample and chemistry/reagent passes through the Restriction Structure (e.g., terminal wall 175) which may act to further slow the transfer of the solution, thereby causing mixing and incubation of the sample. The sample migrates through the Sample Transport Pad (e.g., transfer pad 165) toward the Lateral Flow Strip (e.g., test strip 185). During this process, the sample may be retained for a period of time by the Sample Transport Pad (e.g., transfer pad 165) by the presence of a restriction zone by a compression of pad or due to chemistry/agents in the pad to retard the flow to act as a timing gate which allows for incubation of the chemistry or reagents. Another iteration of this design may include an additional chemistry pad to introduce additional chemistry to the sample. The design and structure of the Sample Transport Pad (e.g., transfer pad 165) may impart an additional metering aspect. The portion of the Sample Transport Pad (e.g., transfer pad 165) proximal to the Lateral Flow Strip (e.g., test strip 185) may be in contact with or suspended above the Sample Application Zone (e.g., sample transfer zone 150) of the test strip. The sample/reagent mixture comes in contact with and is absorbed into a Lateral Flow Strip (e.g., test strip 185) and migrates along the strip, interacting at the various reaction zones on the test strip. An alternate mechanism may be integrated into the housing which holds the Sample Transport Pad (e.g., transfer pad 165) in place above the test strip and allows the sample to be transferred to the Sample Application Zone (e.g., sample transfer zone 150) only after a mechanical or chemical interaction to allow contact with the Sample Application Zone (e.g., sample transfer zone 150). The mechanism controls the flow of the sample assuring the sample remains at the Sample Application Zone (e.g., sample transfer zone 150). In addition, the strip housing may include an area surrounding the Sample Application (e.g., sample transfer zone 150) which acts as a reservoir to collect any excess sample that may build up at the application zone. The Lateral Flow Test Strip (e.g., test strip 185) may contain various elements including, but not limited to nitrocellulose membrane, polyesters pads, glass fiber pads, absorbent pads, dried buffering chemicals, dried conjugated reporter molecule. At this point the device resembles assembly 300 illustrated in FIG. 13 to FIG. 15.

Interrogation of the Lateral Flow Strip—Once the sample has migrated up the test strip and the strip/sample mixture reaction time has been achieved, the reaction zones will be interrogated either visually or by a reader system through the Results Viewing Window (e.g., window 110) to determine results.

### Methods of Manufacture

A component described herein, e.g., a sample collector component, test device component, test device housing component (i.e., anterior member, posterior member) and test device cartridge component (i.e., including a cartridge housing and terminal projection), may be manufactured by any suitable process. Non-limiting examples of manufacturing processes include thermoforming, vacuum forming, pressure forming, plug-assist forming, reverse-draw thermoforming, matched die forming, extrusion, casting and injection molding.

In certain implementations, certain portions of a component described herein are manufactured in one process and then one or more pads manufactured in a separate process are joined to the component. For example, handle and stem portions of a sample collection device component described herein (e.g., component 200, 500, 800 and 1100) can be manufactured as a component by one process and then a sample pad (e.g., sample pad 230, 530, 830, 1130) manufactured by a separate process can be joined to the stem portion. In another example, a cartridge of a test device can be manufactured as a component by one process (e.g., cartridge 170 of test device 100, cartridge 470 of test device 400, cartridge 770 of test device 700 and cartridge 1070 of test device 1000) and then a transfer pad (e.g., transfer pad 165, 465, 765 and 1065) and one or more optional cartridge housing pads (e.g., cartridge housing pads 145, 146, 1045, 1046) each manufactured by a separate process can be joined to the cartridge. In another example, a test device housing posterior member can be manufactured as a component by one process (e.g., posterior member 102, 402, 702, 1002) and a test strip (e.g., test strip 185, 485, 785, 1085) manufactured by a separate process can be joined to the posterior member.

A component described herein often is of a unitary construction and is molded from one material. A unitary component often is molded from one material (e.g., a moldable polymer comprising polypropylene), and the entire component is of the same material. A unitary component manufactured from a first material sometimes includes a pad constructed from the first material or a second material, where the first material (e.g., polypropylene) is different than the second material (e.g., polyethylene). A component sometimes is of a multi-part construction, and two or more parts sometimes are molded separately (e.g., double-shot component). A multi-part component sometimes includes a region manufactured from a first moldable material and another region manufactured from a second moldable material, where the second moldable material sometimes has a greater elasticity than the first moldable material. A multi-part component, for example, sometimes includes a region manufactured from a first moldable material and another region manufactured from a second moldable material, where the second moldable material contains an elastomer, and the first moldable material contains no elastomer, a different elastomer than the elastomer in the second moldable material or a lower content of the same elastomer as in the second moldable material. A unitary component or a multi-part component sometimes includes no elastomer.

Some or all elements of a component sometimes include, or are manufactured from, a suitable polymer or polymer mixture. Non-limiting examples of polymers include polyethylene (PE), low density polyethylene (LDPE), high-density polyethylene (HDPE), polypropylene (PP), high impact polystyrene (HIPS), polyvinyl chloride (PVC), polyethylene terephthalate (PET), amorphous polyethylene terephthalate (APET), acrylonitrile butadiene styrene (ABS) and polycarbonate (PC). One or more elements of a component can include, or can be manufactured from, a recyclable material and/or degradable material (e.g., a bio-degradable material), non-limiting examples of which are disclosed in International Application no. PCT/US2009/063762 filed on Nov. 9, 2009 and published as WO 2010/054337 on May 14, 2010. One or more elements of a component, in some embodiments, include an anti-microbial agent, non-limiting examples of which are disclosed in International Application no. PCT/US2009/047541 filed on Jun. 16, 2009 and published as WO 2010/008737 on Jan. 10, 2010 (e.g., antimicrobial metal (e.g., silver)).

A component described herein sometimes is manufactured by a method that includes: dispensing a molten polymer into a cavity of a mold configured to mold a component described herein, permitting the polymer in the cavity to cool, and releasing the formed component from the mold after cooling. The mold sometimes includes a metal, and sometimes the mold is manufactured from a metal. The metal sometimes includes one or more of aluminum, zinc, steel or a steel alloy. Non-limiting examples of a polymer is provided herein. In certain embodiments, the molding process is an injection molding process.

Also provided herein in certain embodiments is a mold for manufacturing a component described herein by a molding process (e.g., injection molding process), which includes a body that forms exterior surfaces of the component and a member that forms interior surfaces of the component. A mold sometimes includes one or more core components that form interior surfaces of the component (e.g., core pin component).

A component sometimes is manufactured by an injection molding process. Injection molding is a manufacturing process for producing objects from thermoplastic (e.g., nylon, polypropylene, polyethylene, polystyrene and the like, for example) or thermosetting plastic (e.g., epoxy and phenolics, for example) materials. A plastic material (e.g., a polymer material) of choice often is fed into a heated barrel, mixed, and forced into a mold cavity where it cools and hardens to the configuration of the mold cavity. The melted material sometimes is forced or injected into the mold cavity, through openings (e.g., a sprue), under pressure. A pressure injection method often ensures the complete filling of the mold with the melted plastic. After the mold cools, mold portions are separated, and the molded object is ejected.

A plastic with higher flow and lower viscosity sometimes is selected for use in an injection molding process. Non-limiting examples of plastics with higher flow and lower viscosity include any suitable moldable material having one or more of the following properties: a melt flow rate (230 degrees Celsius at 2.16 kg) of about 30 to about 75 grams per 10 minutes using an ASTM D 1238 test method; a tensile strength at yield of about 3900 to about 5000 pounds per square inch using an ASTM D 638 test method; a tensile elongation at yield of about 7 to about 14% using an ASTM D 638 test method; a flexural modulus at 1% sectant of about 110,000 to about 240,000 pounds per square inch using an ASTM D 790 test method; a notched Izod impact strength (23 degrees Celsius) of about 0.4 to about 4.0 foot pounds per inch using an ASTM D 256 test method; and/or a heat deflection temperature (at 0.455 MPa) of about 160 degrees to about 250 degrees Fahrenheit using an ASTM D 648 test method. Non-limiting examples of materials that can be used include polypropylene, polystyrene, polyethylene, acrylonitrile butadiene styrene, polycarbonate, the like, and mixtures thereof. In some embodiments, additional additives can be included in the plastic or mold to impart additional properties to the final product (e.g., anti-microbial, degradable, anti-static properties). A component can be injection molded as a unitary construct.

A mold often is configured to retain molten plastic in a geometry that yields the desired product upon cooling of the plastic. Injection molds sometimes are made of two or more parts. Molds typically are designed so that the molded part reliably remains on the ejector side of the mold after the mold opens, after cooling. The molded part may fall freely away from the mold when ejected from ejector side of the mold. In some embodiments, an ejector pushes the molded part from the ejector side of the mold.

Thus, in certain aspects, provided is a method for manufacturing a sample collection device component described herein, or portion thereof, that includes: injecting a liquid polymer into a mold; hardening the polymer in the mold, where the sample collection device or portion thereof is formed in the mold; and separating the sample collection device or portion thereof from the mold. A portion of a sample collection device sometimes includes or consists of the handle and stem.

In certain aspects, provided is a method for manufacturing a sample collection device described herein that includes: providing a portion of the sample collection device describe herein that includes the handle and stem, and providing the sample pad and joining the sample pad to the stem. In certain instances, joining the sample pad to the stem can include inserting a portion of the sample pad into the interior of the stem. The portion of the sample collection device that includes the handle and stem sometimes is manufactured by a manufacturing process described herein.

Provided in certain aspects is a method for manufacturing a test device component described herein that includes: injecting a liquid polymer into a mold; hardening the polymer in the mold, wherein the test device component or portion thereof is formed in the mold; and separating the test device component or portion thereof from the mold. A test device component sometimes is a test device housing anterior member, test device housing proximal member or cartridge containing a cartridge housing member and terminal projection member.

Also provided in certain aspects is a method for manufacturing a test device component described herein, that includes: providing a test device housing posterior member and a test device housing anterior member; contacting a cartridge (e.g., a cartridge that includes a cartridge housing member and terminal projection) with the test device housing posterior member and/or the test device housing anterior member; and joining the test device posterior member and the test device anterior member, whereby the interior cartridge is contained within the test device housing interior. In certain instances, the cartridge provided contains the transfer pad and optionally contains one or more cartridge housing pads. In certain implementations, the method includes providing a test strip and joining the test strip with the test device housing posterior member and/or the test device housing anterior member prior joining the test device posterior member and the test device anterior member. In certain instances, the test device housing posterior member, the test device housing anterior member and/or the interior cartridge are manufactured according to a method described herein.

In certain aspects, provided is a mold for manufacturing a sample collection device described herein or portion thereof (e.g., a component having the handle and stem). In certain aspects, provided herein is a mold for manufacturing a test device component described herein (e.g., test device housing anterior member, test device housing posterior member, cartridge including the cartridge housing and terminal extension) or portion thereof.

### Certain Implementations

Following are non-limiting examples of certain implementations of the technology.

A1. A sample collection device comprising: a handle, a stem in connection with the handle, and an absorbent sample pad in connection with the stem, wherein:
the sample pad comprises a proximal surface, a distal surface and an exterior side surface between the proximal surface and the distal surface; and
at least a portion of the sample pad extends from the stem.

A2. The sample collection device of embodiment A1, wherein:
the stem comprises an interior; and
a portion of the sample pad is disposed within the stem interior.

A3. The sample collection device of embodiment A1 or A2, wherein a property of the sample pad changes upon contact with a fluid.

A4. The sample collection device of embodiment A3, wherein the sample pad comprises a colormetric agent that shifts color upon contact with fluid.

A5. The sample collection device of embodiment A4, wherein the colormetric agent is disposed at a zone of the sample pad.

A6. The sample collection device of embodiment A5, wherein the zone is a transverse band circumferentially disposed in or on the sample pad.

A7. The sample collection device of embodiment A6, wherein a portion or all of the zone is disposed at the proximal surface of the sample pad.

A8. The sample collection device of embodiment A3, wherein the sample pad or portion thereof swells when contacted with a fluid.

A9. The sample collection device of any one of embodiments A1-A8, wherein the handle and/or the stem, or portion thereof, is transparent or translucent.

A10. The sample collection device of any one of embodiments A1-A9, comprising a sealing member disposed on the stem.

A11. The sample collection device of embodiment A10, wherein the sample collection device comprises a first sealing member and a second sealing member each disposed on the stem.

A12. The sample collection device of embodiment A11, wherein the first sealing member and the second sealing member are o-rings.

A13. The sample collection device of any one of embodiments A1-A12, comprising a lock member disposed on the stem.

A14. The sample collection device of embodiment A13, wherein the lock member is disposed on the stem between the handle and the sealing member.

A15. The sample collection device of embodiment A13 or A14, wherein the lock member is configured to engage a lock member counterpart disposed in a test device to which the sample collection device is configured to join.

A16. The sample collection device of any one of embodiments A13-A15, wherein the lock member is an annular projection.

A16.1. The sample collection device of any one of embodiments A13-A15, wherein the lock members is a threaded member.

A16.2. The sample collection device of embodiment A16.1, wherein:
the handle comprises an interior surface spaced from an exterior surface of the stem;
the interior surface spaced from the exterior surface of the stem comprises the threaded member.

A17. The sample collection device of any one of embodiments A1-A16.2, wherein:
the handle of the sample collection device is a tubular handle or comprises a tubular portion, comprising a handle proximal terminus, a handle distal terminus and a handle wall between the handle proximal terminus and the handle distal terminus;
the stem is a tubular stem or comprises a tubular portion, comprising a stem proximal terminus, a stem distal terminus, a stem wall between the stem proximal terminus and the stem distal terminus and a tubular stem interior;
the stem proximal terminus is in connection with the handle distal terminus; and
the sample pad is a cylindrical pad or comprises a cylindrical portion.

A18. The sample collection device of embodiment A17, wherein:
a first portion of the sample pad side is inserted within the tubular stem interior; and
a second portion of the sample pad side extends from the stem distal terminus.

A19. The sample collection device of any one of embodiments A1-A18, wherein the sample pad absorbs a maximum fluid volume of about 0.1 milliliters to about 10 milliliters.

A20. The sample collection device of embodiment A19, wherein the sample pad absorbs a maximum liquid volume of about 1 milliliter to about 2 milliliters.

A21. The sample collection device of embodiment A20, wherein the sample pad has a density of about 0.01 grams/cubic centimeter (g/cc) to about 0.5 g/cc.

A22. The sample collection device of any one of embodiments A1-A21, wherein the sample pad comprises (i) polyethylene, or (ii) polypropylene, or (iii) polyethylene and polypropylene.

A23. The sample collection device of any one of embodiments A1-A22, wherein the sample pad has an average pore size of about 5 micrometers to about 60 micrometers.

A24. The sample collection device of any one of embodiments A1-A23, wherein the sample pad comprises one or more reagents.

A25. The sample collection device of embodiment A24, wherein the one or more reagents are chosen from a buffering agent, detergent, protein, enzyme, antibody or antigen-binding fragment thereof, antigen, binding pair member, bulking agent, water-absorbing agent, saccharide, polysaccharide, nucleic acid amplification reagent, nucleic acid and aptamer.

A26. The sample collection device of embodiment A24 or A25, wherein the one or more reagents are distributed in all or a portion of the sample pad.

A27. The sample collection device of any one of embodiments A1-A26, wherein the handle and stem independently comprise polypropylene, polyethylene, acrylonitrile butadiene styrene, or a combination of two or more of the foregoing.

B1. A test device, comprising:
a hollow test device housing comprising a proximal terminus, a distal terminus, a sidewall between the proximal terminus and distal terminus, an interior, and a window disposed in the sidewall;
a cartridge disposed within the test device housing, the cartridge comprising:
   a hollow cartridge housing comprising a proximal terminus distally disposed from, and adjacent to, the test device housing proximal terminus, a distal terminus, a sidewall disposed between the proximal terminus and the distal terminus, an interior, and a terminal wall disposed at the distal terminus, and
   a hollow terminal projection member extending from the terminal wall of the cartridge housing, the terminal projection member comprising a proximal terminus disposed on the terminal wall, a distal terminus opposite the terminal wall, a sidewall disposed between the proximal terminus and distal terminus, and an interior;
a porous transfer pad disposed within the terminal projection member of the cartridge; and
a lateral flow strip disposed in the test device housing adjacent to the transfer pad and distally disposed from the cartridge, the strip comprising a proximal surface, a distal surface, and an anterior surface and an opposite posterior surface each disposed between the proximal surface and the distal surface; wherein the window in the test device housing opposes a portion of the strip anterior side.

B2. The test device of embodiment B1, wherein a portion of the transfer pad is disposed within the cartridge terminal projection member and another portion of the transfer pad extends from the distal terminus of the cartridge terminal projection member.

B3. The test device of embodiment B1 or B2, wherein:
the cartridge housing sidewall comprises an interior surface; and
the cartridge housing terminal wall comprises an interior surface comprising a perimeter coextensive with the cartridge sidewall interior surface.

B4. The test device of any one of embodiments B1-B3, wherein the cartridge terminal wall comprises one or more bores.

B5. The test device of embodiment B4, wherein:
the cartridge terminal projection member comprises an interior; and
the one or more bores in the cartridge terminal wall each comprise a distally disposed aperture within the interior of the terminal projection member.

B6. The test device of embodiment B5, wherein:
the transfer pad comprises a proximal surface and a distal surface, and
the proximal surface of the transfer pad abuts an exterior surface of the cartridge terminal wall and the one or more bores in the terminal wall.

B7. The test device of embodiment B5 or B6, wherein:
the transfer pad comprises an anterior side surface and an opposite posterior side surface each between the proximal surface and the distal surface of the pad; and
the anterior side surface and the posterior side surface of the transfer pad each contact an interior surface of the cartridge terminal projection member.

B8. The test device of any one of embodiments B1-B7, wherein the transfer pad is disposed within the cartridge terminal projection member by an interference fit.

B9. The test device of embodiment B7 or B8, wherein a portion of the anterior side surface of the transfer pad contacts a portion of the anterior surface of the strip.

B10. The test device of embodiment B7 or B8, wherein a portion of the distal surface of the transfer pad contacts a portion of the proximal surface of the strip.

B11. The test device of any one of embodiments B1-B10, wherein:
the lateral flow strip comprises a lateral length and a perpendicular transverse width;
the transfer pad comprises a lateral length and a perpendicular transverse width; and
the transfer pad width is substantially the same or the same as the lateral flow strip width.

B12. The test device of any one of embodiments B1-B11, wherein the strip is retained in the test device housing by one or more strip retaining members disposed on an interior surface of the test device housing sidewall.

B13. The test device of embodiment B12, wherein the one or more strip retaining members extend from a posterior sidewall interior surface of the test device housing sidewall.

B14. The test device of embodiment B12 or B13, wherein the strip retaining members comprise (i) one or more laterally disposed ribs, or (ii) one or more containment posts, or (iii) a combination of (i) and (ii).

B15. The test device of embodiment B14, wherein the posterior surface of the strip contacts a proximal edge of each of the one or more laterally disposed ribs of the test device housing.

B16. The test device of embodiment B14 or B15, wherein the strip comprises a laterally disposed edge on each side of the posterior surface and each laterally disposed edge of the strip contacts one or more containment posts of the test device housing.

B17. The test device of any one of embodiments B9-B16, wherein:
the test device housing comprises an anterior sidewall interior surface and an extension bar extending from the anterior sidewall interior surface comprising a posterior edge; and
the posterior edge of the extension bar, or portion of the posterior edge, contacts a portion of the anterior surface of the transfer pad.

B18. The test device of embodiment B17, wherein the posterior edge of the extension bar, or portion of the posterior edge, contacts a portion of the anterior surface of the transfer pad opposite the anterior surface of the test strip.

B19. The test device of any one of embodiments B1-B18, comprising: (i) a first extension wall disposed on and extending from a posterior sidewall interior surface of the test device housing sidewall, or (ii) a second extension wall disposed on and extending from an anterior interior surface of the test device housing sidewall.

B20. The test device of embodiment B19, comprising (i) a first extension wall disposed on and extending from a posterior sidewall interior surface of the test device housing sidewall and (ii) a second extension wall disposed on and extending from an anterior interior surface of the test device housing sidewall, wherein:
the first extension wall comprises an anterior edge;
the second extension wall comprises a posterior edge; and
the anterior edge of the first extension wall is opposite to and spaced from the posterior edge of the second extension wall.

B21. The test device of embodiment B20, wherein:
the terminal projection comprises an anterior wall and a posterior wall;
the anterior edge of the first extension wall contacts an exterior surface of the terminal projection posterior wall; and
the posterior edge of the second extension wall contacts an exterior surface of the terminal projection anterior wall.

B22. The test device of any one of embodiments B19-B21, comprising a third extension wall and a fourth extension wall each disposed on and extending from a posterior sidewall interior surface of the test device housing sidewall, wherein:
the third extension wall comprises an interior side that opposes an interior side of the fourth lateral extension wall; and
the interior side of the third extension wall contacts a side of the first extension wall and the interior side of the fourth extension wall contacts an opposing side of the first extension wall.

B23. The test device of embodiment B22, wherein:
the first extension wall comprises a major dimension that is transversely disposed;
the third extension wall and the fourth extension wall each comprise a major dimension that is laterally disposed; and
the first extension wall is perpendicular to each of the third extension wall and the fourth extension wall.

B24. The test device of embodiment B23, wherein:
the cartridge terminal projection comprises a sidewall and an opposing sidewall between the anterior wall and the posterior wall;
a portion of the interior side of the third extension wall contacts an exterior surface of the cartridge terminal projection sidewall; and
a portion of the interior side of the fourth extension wall contacts an exterior surface of the cartridge terminal projection opposing sidewall.

B25. The test device of any one of embodiments B1-B24, wherein:
the test device housing comprises a proximal opening rim and an annular recessed surface distally disposed and adjacent to the proximal opening rim; and
the proximal terminus of the cartridge housing contacts the annular recessed surface of the test device housing.

B25.1. The test device of embodiment B25, comprising an annular projection disposed on an interior surface of the test device housing sidewall and distally disposed from the proximal opening rim, the annular projection contacting a portion of an exterior surface of the cartridge housing sidewall.

B25.2. The test device of embodiment B1-B24, wherein:
the cartridge housing comprises an annular collar extending from an exterior surface of the cartridge housing sidewall;
the test device housing comprises a proximal opening rim and an annular recessed surface distally disposed and adjacent to the proximal opening rim; and
the collar contacts the annular recessed surface and the rim of the test device housing.

B26. The test device of any one of embodiments B1-B25.2, comprising post members each disposed on and extending from a posterior sidewall interior surface of the test device housing sidewall, wherein:
a portion of one post member contacts a portion of the distal terminus of the terminal projection of the cartridge; and
a portion of another post member contacts another portion of the distal terminus of the terminal projection of the cartridge.

B27. The test device of embodiment B26, wherein:
the third extension wall and the fourth extension wall each comprise a distal side;
one post member is connected to the distal side of the third extension wall; and
another post member is connected to the distal side of the fourth extension wall.

B28. The test device of any one of embodiments B22-B27, wherein:
the third extension wall and the fourth extension wall each comprise a proximal side;
the proximal side of the third extension wall contacts a portion of the terminal wall of the cartridge housing; and
the proximal side of the fourth extension wall contacts another portion of the terminal wall of the cartridge housing.

B29. The test device of any one of embodiments B1-B28, wherein (i) the test device housing is a tube, or (ii) the cartridge housing is a tube, or (iii) the cartridge terminal projection is a tube, or (iv) a combination of two or more of (i), (ii) and (iii).

B30. The test device of embodiment B29, wherein a first region of the test device housing is a tube comprising a circular or ovoid cross section.

B31. The test device of embodiment B30, wherein the first region of the test device housing is a cylindrical tube.

B32. The test device of embodiment B30 or B31, wherein the first region of the test device housing is co-terminal with the proximal terminus of the test device housing.

B33. The test device of any one of embodiments B30-B32, wherein a second region of the test device housing is a tube comprising a lateral planar cylindrical segment region.

B34. The test device of embodiment B33, wherein the second region is co-terminal with the distal terminus of the test device housing.

B35. The test device of any one of embodiments B30-B34 wherein a third region of the test device housing is a tube comprising a lateral arcoid cylindrical segment region.

B36. The test device of embodiment B35, wherein the third region is disposed between the first region and the second region of the test device housing, and is co-extensive with the first region and the second region of the test device housing.

B37. The test device of any one of embodiments B29-B36, wherein the cartridge housing is a tube comprising a circular or ovoid cross section.

B38. The test device of any one of embodiments B29-B37, wherein the cartridge terminal projection is a tube comprising a polygon cross section.

B39. The test device of any one of embodiments B1-B38, wherein the test device housing comprises a bore and the perimeter of the bore defines the window.

B40. The test device of embodiment B39, wherein the perimeter of the bore is ovoid or polygonal.

B41. The test device of any one of embodiments B35-B40, wherein the window is disposed in portion of the second region and an adjacent portion of the third region.

B42. The test device of any one of embodiments B1-B41, wherein the test device housing comprises a lock member counterpart to a lock member of a sample collection device configured to join with the test device.

B43. The test device of embodiment B42, wherein the lock member counterpart is an annular groove.

B43.1. The test device of embodiment B42, wherein the lock member counterpart is a threaded member.

B43.2. The test device of embodiment B43.1, wherein:
the cartridge housing comprises a hollow shank extending from the cartridge housing proximal terminus; and
the lock member counterpart is a threaded member disposed on the shank.

B44. The test device of embodiment B43, wherein:
the cartridge housing comprises an annular recessed edge adjacent to the proximal terminus;
the test device housing comprises a proximal opening rim and an annular recessed surface adjacent to the proximal opening rim; and
the annular groove is disposed between the annular recessed edge of the cartridge housing and the annular recessed surface of the test device housing.

B45. The test device of any one of embodiments B1-B44, comprising a sealing member disposed on an interior surface of the test device housing and/or an interior surface of the cartridge housing.

B46. The test device of any one of embodiments B1-B44, comprising one more porous cartridge pads disposed in an interior portion of the cartridge housing.

B47. The test device of embodiment B46, wherein:
each of the one or more porous cartridge pads comprises a proximal surface, a distal surface and a side surface disposed between the proximal surface and the distal surface;
the terminal wall of the cartridge housing comprises an interior surface; and
the distal surface of one of the cartridge pad contacts the interior surface of the cartridge housing terminal wall.

B48. The test device of embodiment B46 or B47, comprising two or more porous cartridge pads disposed in a stacked assembly.

B49. The test device of any one of embodiments B46-B48, wherein the transfer pad is in fluid communication with the one or more porous cartridge pads.

B50. The test device of any one of embodiments B47-B49, wherein the proximal surface of the transfer pad is in fluid communication, via the bores in the terminal wall of the cartridge housing, with the distal surface of a cartridge pad.

B51. The test device of any one of embodiments B46-B50, wherein:
the cartridge housing sidewall comprises an interior surface; and
the side surface of each of the one or more cartridge pads contacts a portion of the interior surface of the cartridge housing sidewall.

B52. The test device of any one of embodiments B1-B51, wherein the transfer pad comprises (i) polyethylene, or (ii) polypropylene, or (iii) polyethylene and polypropylene.

B53. The test device of any one of embodiments B1-B52, wherein the transfer pad retains a maximum fluid volume of about 20 microliters to about 1 milliliter.

B54. The test device of embodiment B53, wherein the transfer pad retains a maximum fluid volume of about 50 microliters to about 500 microliters.

B55. The test device of embodiment B54, wherein the transfer pad retains a maximum fluid volume of about 100 microliters to about 300 microliters.

B56. The test device of any one of embodiments B1-B55, wherein the transfer pad has an average pore size of about 2 micrometers to about 100 micrometers.

B57. The test device of any one of embodiments B1-B56, wherein the test device housing and the cartridge independently comprise polypropylene, polyethylene, acrylonitrile butadiene styrene, or a combination of two or more of the foregoing.

B58. The test device of any one of embodiments B1-B57, wherein the test device housing is a two-piece housing comprising an anterior member and a posterior member.

B59. The test device of embodiment B58, wherein the posterior member comprises at least one connector and the anterior member comprises at least one connector counterpart.

B60. The test device of any one of embodiments B1-B59, wherein the cartridge is a unitary member.

B61. The test device of any one of embodiments B46-B60, wherein the one more porous cartridge pads each comprise a circular proximal surface and a circular distal surface.

B62. The test device of any one of embodiments B46-B61, wherein the one or more porous cartridge pads independently comprise (i) polyethylene, or (ii) polypropylene, or (iii) polyethylene and polypropylene.

B63. The test device of any one of embodiments B46-B62, wherein the one or more porous cartridge pads each independently have an average pore diameter of about 0.45 micrometers to about 1 micrometer.

B64. The test device of any one of embodiments B1-B63, wherein the transfer pad and the one or more cartridge pads independently comprise one or more reagents.

B65. The test device of embodiment B64, wherein the one or more reagents are chosen from an analyte binding agent, buffering agent, cell lysis agent, detergent, protein, enzyme, antibody or antigen-binding fragment thereof, antigen, binding pair member, bulking agent, water-absorbing agent, saccharide, polysaccharide, nucleic acid amplification reagent, nucleic acid and aptamer.

B66. The test device of embodiment B64 or B65, wherein the one or more reagents are distributed in all or a portion of the transfer pad and/or the one or more cartridge pads.

B67. The test device of embodiment B66, wherein the transfer pad comprises a water-absorbing agent in region adjacent to the distal surface of the pad.

B68. The test device of any one of embodiments B1-B67, wherein the strip comprises nitrocellulose.

B69. The test device of any one of embodiments B1-B68, wherein the strip comprises one or more reagents.

B70. The test device of any one of embodiments B1-B69, wherein a portion of the transfer pad disposed adjacent to a portion of the test strip are separated.

B71. The test device of embodiment B70, comprising a removable barrier between the portion of the transfer pad and the portion of the test strip adjacent to the transfer pad.

B72. The test device of embodiment B71, wherein:
a portion of the transfer pad posterior surface is disposed opposite of a portion of the test strip anterior surface; and
the removable barrier is disposed between the portion of the transfer pad posterior surface and the portion of the test strip anterior surface opposite to the portion of the transfer pad posterior surface.

B73. The test device of embodiment B71, wherein the removable barrier is a pull tab.

B74. The test device of any one of embodiments B70-B72, wherein a portion of the test device housing comprises a slot through which the removable barrier protrudes and extends from an exterior surface of the test device housing.

B75. The test device of embodiment B70, comprising a button disposed in the test device housing opposite of the portion of the transfer pad disposed adjacent to the portion of the test strip.

B76. The test device of embodiment B75, wherein:
a portion of the transfer pad posterior surface is disposed opposite of a portion of the test strip anterior surface; and
the button is disposed opposite of the portion of the transfer pad.

B77. The test device of embodiment B76, wherein the button is defined by slits in the test device housing.

B78. The test device of embodiment B77, wherein the button is defined by three slits in the test device housing and a portion of the test device housing sidewall opposite one of the slits is a living hinge.

B79. The test device of any one of embodiments B75-B78, wherein the button is disposed on an anterior surface of the test device housing.

B80. The test device of any one of embodiments B75-B79, wherein:
the test device housing comprises an extension bar extending from an interior surface of the button and the test device housing; and
a posterior surface of the extension bar is disposed opposite of the portion of the transfer pad disposed adjacent to the test strip.

B81. The test device of embodiment B80, wherein:
the test device housing comprises a catch; and
the extension bar comprises a catch counterpart disposed adjacent to the catch in the test device housing.

C1. A composition, comprising: a sample collection device of any one of embodiments A1-A27 and a test device of any one of embodiments B1-B81, wherein the sample collection device is separated from the test device.

C2. The composition of embodiment C1, wherein the sample collection device is configured to join with the test device and the sample collection device is not joined and physically separated from the test device in the composition.

C3. The composition of embodiment C1 or C2, wherein:
a lateral distance between the sample collection device handle distal terminus and the sample pad distal terminus is a first lateral distance;
a lateral distance (i) between the test device housing proximal terminus and the interior surface of the cartridge housing terminal wall, or (ii) between the test device housing proximal terminus and a proximal surface of a cartridge housing pad where the cartridge housing contains a cartridge housing pad, is a second lateral distance; and
the first lateral distance is greater than the second lateral distance.

D1. A kit, comprising: a sample collection device of any one of embodiments A1-A27 and a test device of any one of embodiments B1-B81, wherein the sample collection device is separated from the test device.

D2. The kit of embodiment D1, comprising instructions for joining the sample collection device with the test device.

E1. An assembly, comprising: a sample collection device of any one of embodiments A1-A27 and a test device of any one of embodiments B1-B81, wherein the sample collection device is joined with the test device.

E2. The assembly of embodiment E1, wherein:
the test device is distally disposed from the sample collection device handle; and
the sample pad and at least a portion of the stem are contained within the test device.

E3. The assembly of embodiment E2, wherein the sample pad and at least a portion of the stem of the sample collection device are disposed within the interior of the cartridge housing of the test device.

E4. The assembly of any one of embodiments E1-E3, wherein the sample pad is laterally compressed in the assembly relative to the lateral length of the sample pad when the sample collection device and the test device are separate and not joined.

E5. The assembly of embodiment E4, wherein:
a lateral length between the sample pad proximal terminus and the sample pad distal terminus in the sample collection device not joined to the test device is a first lateral distance;
a lateral length between the sample pad proximal terminus and the sample pad distal terminus in the sample collection device joined to the test device is a second lateral distance; and
the first lateral distance is greater than the second lateral distance.

E6. The assembly of embodiment E5, wherein the distal terminus of the sample pad contacts an interior surface of the cartridge housing terminal wall or portion of the interior surface of the cartridge housing terminal wall.

E7. The assembly of embodiment E5, wherein the distal terminus of the sample pad contacts a proximal surface of a cartridge housing pad or portion of the proximal surface of the cartridge housing pad.

E8. The assembly of any one of embodiments E1-E7, wherein:
the sample collection device comprises a lock member disposed on the stem;
the test device comprises a lock member counterpart disposed on an interior surface of the test device; and
the lock member of the sample collection device is engaged with the lock member counterpart of the test device.

E8.1. The assembly of any one of embodiments E1-E7, wherein:
the sample collection device comprises a lock member disposed in the handle;
the test device comprises a lock member counterpart disposed on an exterior surface of the test device; and
the lock member of the sample collection device is engaged with the lock member counterpart of the test device.

E9. The assembly of embodiment E8 or E8.1, wherein the sample collection device is locked to the test device.

E10. The assembly of embodiment E9, wherein the sample collection device is irreversibly locked to the test device.

E11. The assembly of any one of embodiments E8-E10, wherein:
the lock member of the sample collection device is an annular projection;
the lock member counterpart of the test device is an annular groove; and
the annular projection is disposed in the annual groove.

E11.1. The assembly of any one of embodiments E8-E10, wherein:
the lock member of the sample collection device comprises a threaded member; and
the lock member counterpart of the test device comprises a threaded member that engages the threaded member of the sample collection device.

E11.2. The assembly of embodiment E11.1, wherein the threaded member of the sample collection device is disposed on an interior surface of the handle and the threaded member of the test device is disposed on a hollow shank extending from the cartridge and from the proximal terminus of the test device housing.

E12. The assembly of any one of embodiments E1-E11.2, wherein the sample collection device is sealing engaged with the test device.

E13. The assembly of embodiment E12, wherein:
the sample collection device comprises a sealing member disposed at the stem; and
the sealing member is in sealing contact with (i) a sealing member counterpart disposed at an interior wall surface of the test device, or (ii) the interior wall surface of the test device.

E14. The assembly of any one of embodiments E1-E13, wherein the sample pad of the sample collection device comprises sample fluid.

E15. The assembly of embodiment E14, wherein the sample fluid transfers from the sample pad of the sample collection device to the transfer pad in the test device and from the transfer pad to the strip in the test device.

E16. The assembly of embodiment E15, wherein the sample fluid transfers from the sample pad of the sample collection device through one or more bores disposed in the terminal wall of the cartridge housing, to the transfer pad.

E17. The assembly of embodiment E16, wherein the sample fluid transfers from the sample pad of the sample collection device, through one or more cartridge housing pads, then through one or more bores disposed in the terminal wall of the cartridge housing, and then to the transfer pad.

F1. A method for preparing an assembly containing a sample collection device and a test device, comprising: joining a sample collection device of any one of embodiments A1-A27 to a test device of any one of embodiments B1-B81, thereby forming the assembly of any one of embodiments E1-E17.

F1.1. The method of embodiment F1, wherein the sample pad of the collection device is inserted into the interior of the cartridge housing.

F1.2. The method of embodiment F1.1, wherein the stem or portion thereof of the collection device is inserted into the interior of the cartridge housing.

F2. The method of embodiment F1, F1.1 or F1.2, wherein the sample pad of the sample collection device contains sample fluid.

F3. The method of embodiment F2, comprising, prior to joining the sample collection device to the test device, contacting the sample pad of the sample collection device with sample fluid.

F4. The method of embodiment F3, wherein a property of the sample pad of the sample collection device changes upon contact with the sample fluid.

F5. The method of any one of embodiments F1-F4, wherein joining the sample collection device to the test device comprises engaging the lock member of the sample collection device with the lock member counterpart of the test device.

F6. The method of embodiment F5, wherein the lock member and the lock member counterpart are engaged by an interference fit or threaded fit.

F7. The method of embodiment F5 or F6, wherein engaging the lock member and the lock member counterpart emits an audible sound.

F8. The method of any one of embodiments F1-F7, wherein joining the sample collection device to the test device laterally compresses the sample pad.

F9. The method of embodiment F8, wherein the sample pad is laterally compressed when the lock member is engaged with the lock member counterpart.

F10. The method of embodiment F8 or F9, wherein the sample pad is laterally compressed when the sample collection device and the test device are sealing engaged.

F11. The method of any one of embodiments F8-F10, wherein compression of the sample pad expresses sample fluid from the sample pad.

F12. The method of embodiment F11, wherein:
the sample pad retains components in the sample fluid; and
sample fluid expressed from the sample pad comprises an amount of the components less than the amount in the sample fluid applied to the sample pad.

F14. The method of embodiment F13, wherein the sample pad retains high molecular weight mucins and the sample fluid expressed from the sample pad comprises an amount of the high molecular weight mucins lower than the amount in the sample fluid applied to the sample pad.

F15. The method of any one of embodiments F8-F14, wherein sample fluid is expressed from the sample pad to the transfer pad and from the transfer pad to the strip in the test device.

F16. The method of embodiment F15, wherein the sample fluid transfers from the sample pad of the sample collection device through one or more bores disposed in the terminal wall of the cartridge housing, to the transfer pad.

F17. The method of embodiment F15, wherein the sample fluid transfers from the sample pad of the sample collection device, through one or more cartridge housing pads, then through one or more bores disposed in the terminal wall of the cartridge housing, and then to the transfer pad.

F18. The method of embodiment F17, wherein:
at least one of the one or more cartridge housing pads retains one or more components in the input sample fluid; and
sample fluid outputted from the at least one of the one or more cartridge housing pads comprises an amount of the components less than the amount in the input sample fluid.

F19. The method of embodiment F18, wherein the at least one of the one or more cartridge housing pads retains low molecular weight mucins and the sample fluid outputted comprises an amount of the low molecular weight mucins lower than the amount in the input sample fluid.

F20. The method of any one of embodiments F8-F19, wherein one or more of the pads of the sample collection device and the test device comprises one or more reagents that interact with the sample fluid.

F21. The method of embodiment F20, wherein the one or more reagents are in dry form and interact in solution with the sample fluid when the sample fluid enters the pad.

F22. The method of embodiment F20 or F21, wherein at least a portion of the one or more reagents dissolve into the input sample fluid.

F23. The method of any one of embodiments F15-F22, wherein sample fluid transfers from a portion of the transfer pad to a portion of the test strip.

F24. The method of embodiment F23, wherein the sample fluid transfers from a portion of a posterior surface of the transfer pad to a portion of an anterior surface of the test strip opposite of the portion of the transfer pad.

F25. The method of embodiment F23 or F24, wherein the portion of the transfer pad and the portion of the transfer strip are in direct contact prior to joining the sample collection device to the test device.

F26. The method of any one of embodiments F23-F25, wherein:
the portion of the transfer pad and the portion of the test strip are not in direct contact prior to, and after, joining the sample collection device to the test device; and
the portion of the transfer pad and the portion of the test strip are placed in direct contact after the sample collection device is joined to the test device.

F27. The method of embodiment F26, wherein the portion of the transfer pad and the portion of the test strip are placed in direct contact after an amount of time elapses after the sample collection device is joined to the test device.

F28. The method of embodiment F26 or F27, wherein:
the portion of the transfer pad and the portion of the test strip are separated by a removable barrier prior to, and after, joining the sample collection device to the test device; and
the portion of the transfer pad and the portion of the test strip are placed in direct contact after the sample collection device is joined to the test device by removing the removable barrier.

F29. The method of embodiment F26 or F27, wherein:
the portion of the transfer pad and the portion of the test strip are separated prior to, and after, joining the sample collection device to the test device; and
the portion of the transfer pad and the portion of the test strip are placed in direct contact after the sample collection device is joined to the test device by operating a button present in the test device housing.

F30. The method of embodiment F29, wherein:
the button is depressed in the anterior to posterior direction;
a portion of the device housing placed in contact with an anterior surface of the transfer pad opposite of the portion of the test strip after the button is depressed, or a portion of the device housing in contact with an anterior surface of the transfer pad opposite of the portion of the test strip before the button is depressed, causes the portion of the transfer pad to contact the portion of the test strip.

F31. The method of embodiment F30, wherein the portion of the device housing placed in contact with the anterior surface of the transfer pad is an extension bar extending from an interior surface of an anterior portion of the test device housing sidewall.

G1. A method for determining presence or absence and/or amount of an analyte in a sample, comprising:
detecting through the window of the test device of an assembly of any one of embodiments E1-E17, or an assembly prepared by a method of any one of embodiments F1-F31, a detectable signal from the lateral flow strip; and
determining the presence or absence and/or amount of the analyte in the sample from the detectable signal.

G2. The method of embodiment G1, wherein the detectable signal is an optically detectable signal.

G3. The method of embodiment G1 of G2, wherein the signal is detected by an instrument.

G3. The method of embodiment G2, wherein the signal is detected by eye.

H1. A method for manufacturing a sample collection device of any one of embodiments A1-A27, comprising:
injecting a liquid polymer into a mold;
hardening the polymer in the mold, wherein the sample collection device or portion thereof is formed in the mold;
separating the sample collection device or portion thereof from the mold.

H2. The method of embodiment H1, wherein the portion of the sample collection device comprises the handle and stem.

H3. The method of embodiment H1, wherein the portion of the sample collection device consists of the handle and stem.

I1. A method for manufacturing a sample collection device of any one of embodiments A1-A27, comprising:
providing a portion of the sample collection device of any one of embodiments A1-A27 comprising the handle and stem; and
providing the sample pad and joining the sample pad to the stem.

I2. The method of embodiment I1, comprising inserting a portion of the sample pad into the interior of the stem.

I3. The method of embodiment I1 or I2, comprising manufacturing the portion of the sample collection device according to the method of any one of embodiments H1-H3.

J1. A method for manufacturing a test device of any one of embodiments B1-B81, comprising:
injecting a liquid polymer into a mold;
hardening the polymer in the mold, wherein the test device or portion thereof is formed in the mold;
separating the test device or portion thereof from the mold.

J2. The method of embodiment J1, wherein the portion of the test device comprises the test device housing or portion thereof.

J3. The method of embodiment J3, wherein the portion of the test device housing comprises a test device housing posterior member or a test device housing anterior member.

J4. The method of embodiment J1, wherein the portion of the test device comprises the interior cartridge.

K1. A method for manufacturing a test device of any one of embodiments B1-B81, comprising:
providing a test device housing posterior member and a test device housing anterior member;
contacting the cartridge with the test device housing posterior member and/or the test device housing anterior member; and
joining the test device posterior member and the test device anterior member, whereby the interior cartridge is contained within the test device housing interior.

K2. The method of embodiment K1, wherein the cartridge provided contains the transfer pad and optionally contains one or more cartridge housing pads.

K3. The method of embodiment K1 or K2, comprising providing the test strip and joining the test strip with the test device housing posterior member and/or the test device housing anterior member prior joining the test device posterior member and the test device anterior member, whereby the interior cartridge is contained within the test device housing interior.

K4. The method of any one of embodiments K1-K3, comprising manufacturing the test device housing posterior member, the test device housing anterior member and/or the interior cartridge according to the method of any one of embodiments J1-J4.

L1. A mold for manufacturing a sample collection device of any one of embodiments A1-A27 or portion thereof.

L2. A mold for manufacturing a test device of any one of embodiments B1-B81 or portion thereof.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claims that follow.

What is claimed is:

1. A composition, comprising a sample collection device and a test device separated from the sample collection device,
the sample collection device comprising:
a handle, a stem in connection with the handle, and an absorbent sample pad in connection with the stem, wherein:
the sample pad comprises a proximal surface, a distal surface and an exterior side surface between the proximal surface and the distal surface; and
at least a portion of the sample pad extends from the stem;
the test device comprising:
a hollow test device housing comprising a proximal terminus, a distal terminus, a sidewall between the proximal terminus and distal terminus, an interior, and a window disposed in the sidewall;
a cartridge disposed within the test device housing, the cartridge comprising:
a hollow cartridge housing comprising a proximal terminus distally disposed from, and adjacent to, the test device housing proximal terminus, a distal terminus, a sidewall disposed between the proximal terminus and the distal terminus, an interior, and a terminal wall disposed at the distal terminus, and
a hollow terminal projection member extending from the terminal wall of the cartridge housing, the terminal projection member comprising a proximal terminus disposed on the terminal wall, a distal terminus opposite the terminal wall, a sidewall disposed between the proximal terminus and distal terminus, and an interior;
a porous transfer pad disposed within the terminal projection member of the cartridge; and
a lateral flow strip disposed in the test device housing adjacent to the transfer pad and distally disposed from the cartridge, the strip comprising a proximal surface, a distal surface, an anterior surface and an opposite posterior surface each disposed between the proximal surface and the distal surface; wherein the window in the test device housing opposes a portion of the strip anterior side.

2. The composition of claim 1, wherein a portion of the transfer pad is disposed within the cartridge terminal projection member and another portion of the transfer pad extends from the distal terminus of the cartridge terminal projection member.

3. The composition of claim 1, wherein:
the cartridge housing sidewall comprises an interior surface;
the cartridge housing terminal wall comprises an interior surface comprising a perimeter coextensive with the cartridge sidewall interior surface; and
the cartridge terminal wall comprises one or more bores.

4. The composition of claim 3, wherein:
the cartridge terminal projection member comprises an interior; and
the one or more bores in the cartridge terminal wall each comprise a distally disposed aperture within the interior of the terminal projection member.

5. The composition of claim 4, wherein:
the transfer pad comprises a proximal surface and a distal surface, and
the proximal surface of the transfer pad abuts an exterior surface of the cartridge terminal wall and the one or more bores in the terminal wall.

6. The composition of claim 5, wherein a ratio of (i) a combined surface area of the one or more bores, to (ii) a surface area of the interior surface of the terminal wall of the cartridge housing, is about 0.08 to about 0.5.

7. The composition of claim 1, wherein:
the test device housing comprises an anterior sidewall interior surface and an extension bar extending from the anterior sidewall interior surface comprising a posterior edge; and
the posterior edge of the extension bar, or portion of the posterior edge, contacts a portion of the anterior surface of the transfer pad opposite the anterior surface of the test strip.

8. The composition of claim 1, wherein:
the test device comprises: (i) a first extension wall disposed on and extending from a posterior sidewall interior surface of the test device housing sidewall and (ii) a second extension wall disposed on and extending from an anterior interior surface of the test device housing sidewall, wherein:
the first extension wall comprises an anterior edge;
the second extension wall comprises a posterior edge;
the anterior edge of the first extension wall is opposite to and spaced from the posterior edge of the second extension wall;
the terminal projection comprises an anterior wall and a posterior wall;
the anterior edge of the first extension wall contacts an exterior surface of the terminal projection posterior wall; and
the posterior edge of the second extension wall contacts an exterior surface of the terminal projection anterior wall.

9. The composition of claim 1, comprising one more porous cartridge pads disposed in an interior portion of the cartridge housing.

10. The composition of claim 9, wherein:
each of the one or more porous cartridge pads comprises a proximal surface, a distal surface and a side surface disposed between the proximal surface and the distal surface;
the terminal wall of the cartridge housing comprises an interior surface; and
the distal surface of one cartridge pad contacts the interior surface of the cartridge housing terminal wall.

11. The composition of claim 1, wherein a property of the sample pad changes upon contact with a fluid.

12. The composition of claim 1, wherein:
the sample collection device comprises a sealing member disposed on the stem;
the sample collection device comprises a lock member;
the test device comprises a lock member counterpart configured to engage the lock member of the sample collection device.

13. The composition of claim 1, wherein:
the handle of the sample collection device is a tubular handle comprising a handle proximal terminus, a handle distal terminus and a handle wall between the handle proximal terminus and the handle distal terminus;
the stem is a tubular stem, comprising a stem proximal terminus, a stem distal terminus, a stem wall between the stem proximal terminus and the stem distal terminus and a tubular stem interior;
the stem proximal terminus is in connection with the handle distal terminus;
the sample pad is a cylindrical pad; and
the handle, the stem and the sample pad are concentrically disposed.

14. The composition of claim 13, wherein:
a first portion of the sample pad side is disposed within the tubular stem interior; and
a second portion of the sample pad side extends from the stem distal terminus.

15. The composition of claim 1, wherein:
a lateral distance between the sample collection device handle distal terminus and the sample pad distal terminus is a first lateral distance;
a lateral distance (i) between the test device housing proximal terminus and the interior surface of the cartridge housing terminal wall, or (ii) between the test device housing proximal terminus and a proximal surface of a cartridge housing pad where the cartridge housing contains a cartridge housing pad, is a second lateral distance; and
the first lateral distance is greater than the second lateral distance.

16. The composition of claim 1, wherein:
the lateral length of the sample pad is compressible;
the lateral length between the sample pad proximal terminus and the sample pad distal terminus in the sample collection device not joined to the test device is a first lateral distance;
a lateral length between the sample pad proximal terminus and the sample pad distal terminus in the sample collection device joined to the test device is a second lateral distance;
the first lateral distance is greater than the second lateral distance; and
the second lateral distance is about 60% to about 85% of the first lateral distance.

17. The composition of claim 1, wherein the sample pad comprises one or more of:
(i) a retention volume of about 0.25 milliliters to about 2.5 milliliters;
(ii) a density of about 0.05 g/cc to about 0.8 g/cc;
(iii) an average pore diameter of about 10 micrometers to about 50 micrometers;
(iv) a minimum pore diameter of about 0.5 micrometers to about 5 micrometers;
(v) a maximum pore diameter of about 50 micrometers to about 200 micrometers;
(vi) polyethylene, or polypropylene, or polyethylene and polypropylene;
(vii) a length of about 25 millimeters (mm) to about 45 mm; and
(viii) an outer diameter of about 1 mm to about 15 mm.

18. The composition of claim 1, wherein the transfer pad comprises one or more of:
(i) a retention volume of about 100 microliters to about 300 microliters;
(ii) an average pore diameter of about 10 micrometers to about 40 micrometers;
(iii) a minimum pore diameter of about 0.5 micrometers to about 5 micrometers;
(iv) a maximum pore diameter of about 40 micrometers to about 80 micrometers;
(v) polyethylene, or polypropylene, or polyethylene and polypropylene;
(vi) a lateral length of about 8 millimeters (mm) to about 16 mm;
(vii) a transverse width of about 4 mm to about 10 mm; and
(viii) a longitudinal height of about 1 mm to about 5 mm.

19. An assembly, comprising a sample collection device joined to a test device,
the sample collection device comprising:
a handle, a stem in connection with the handle, and an absorbent sample pad in connection with the stem, wherein:
the sample pad comprises a proximal surface, a distal surface and an exterior side surface between the proximal surface and the distal surface; and
at least a portion of the sample pad extends from the stem;
the test device comprising:
a hollow test device housing comprising a proximal terminus, a distal terminus, a sidewall between the proximal terminus and distal terminus, an interior, and a window disposed in the sidewall;
a cartridge disposed within the test device housing, the cartridge comprising:
a hollow cartridge housing comprising a proximal terminus distally disposed from, and adjacent to, the test device housing proximal terminus, a distal terminus, a sidewall disposed between the proximal terminus and the distal terminus, an interior, and a terminal wall disposed at the distal terminus, and
a hollow terminal projection member extending from the terminal wall of the cartridge housing, the terminal projection member comprising a proximal terminus disposed on the terminal wall, a distal terminus opposite the terminal wall, a sidewall disposed between the proximal terminus and distal terminus, and an interior;
a porous transfer pad disposed within the terminal projection member of the cartridge; and
a lateral flow strip disposed in the test device housing adjacent to the transfer pad and distally disposed from the cartridge, the strip comprising a proximal surface, a distal surface, and an anterior surface and an opposite posterior surface each disposed between the proximal surface and the distal surface;
wherein the window in the test device housing opposes a portion of the strip anterior side.

20. The assembly of claim 19, wherein the sample pad and at least a portion of the stem of the sample collection device are disposed within the interior of the cartridge housing of the test device.

21. The assembly of claim 20, wherein the sample pad is laterally compressed in the assembly relative to the lateral length of the sample pad when the sample collection device and the test device are separate and not joined.

22. The assembly of claim 21, wherein the distal terminus of the sample pad contacts an interior surface of the cartridge housing terminal wall or portion of the interior surface of the cartridge housing terminal wall, or the distal terminus of the sample pad contacts a proximal surface of a cartridge housing pad or portion of the proximal surface of the cartridge housing pad.

23. The assembly of claim 21, wherein:
the sample collection device comprises a lock member;
the test device comprises a lock member counterpart engaged with the lock member of the sample collection device; and
the sample collection device and the test device are sealingly engaged.

24. The assembly of claim 23, wherein:
the sample pad comprises sample fluid; and
the sample fluid transfers from the sample pad of the sample collection device through one or more bores disposed in the terminal wall of the cartridge housing, to the transfer pad.

25. A method for preparing an assembly of claim 19, comprising: joining the sample collection device to the test device.

26. A method for determining presence or absence and/or amount of an analyte in a sample, comprising:
detecting through the window of the test device of an assembly of claim 15 a detectable signal from the lateral flow strip; and
determining the presence or absence and/or amount of the analyte in the sample from the detectable signal.

27. A sample collection device comprising: a handle, a stem in connection with the handle, and an absorbent sample pad in connection with the stem, wherein:
the sample pad comprises a proximal surface, a distal surface and an exterior side surface between the proximal surface and the distal surface; and
at least a portion of the sample pad extends from the stem.

28. A test device, comprising:
a hollow test device housing comprising a proximal terminus, a distal terminus, a sidewall between the proximal terminus and distal terminus, an interior, and a window disposed in the sidewall;
a cartridge disposed within the test device housing, the cartridge comprising:
a hollow cartridge housing comprising a proximal terminus distally disposed from, and adjacent to, the test device housing proximal terminus, a distal terminus, a sidewall disposed between the proximal terminus and the distal terminus, an interior, and a terminal wall disposed at the distal terminus, and a hollow terminal projection member extending from the terminal wall of the cartridge housing, the terminal projection member comprising a proximal terminus disposed on the terminal wall, a distal terminus opposite the terminal wall, a sidewall disposed between the proximal terminus and distal terminus, and an interior;
a porous transfer pad disposed within the terminal projection member of the cartridge; and
a lateral flow strip disposed in the test device housing adjacent to the transfer pad and distally disposed from the cartridge, the strip comprising a proximal surface, a distal surface, and an anterior surface and an opposite posterior surface each disposed between the proximal surface and the distal surface; wherein the window in the test device housing opposes a portion of the strip anterior side.
